(19) United States
(12) Patent Application Publication
(10) Pub. No.: US 2025/0019680 A1
(43) Pub. Date: Jan. 16, 2025

(54) ENGINEERED CAS13 FOR ULTRASENSITIVE NUCLEIC ACID DETECTION

(71) Applicant: William Marsh Rice University, Houston, TX (US)

(72) Inventors: Xue GAO, Houston, TX (US); Jie YANG, Houston, TX (US); Yang GAO, Houston, TX (US)

(73) Assignee: William Marsh Rice University, Houston, TX (US)

(21) Appl. No.: 18/715,895

(22) PCT Filed: Dec. 2, 2022
(86) PCT No.: PCT/US2022/051708

§ 371 Date: Jun. 3, 2024

Related U.S. Application Data
(60) Provisional application No. 63/285,304, filed on Dec. 2, 2021.

(51) Int. Cl. C12N 9/22; C12N 15/11; C12Q 1/70

(52) U.S. Cl. CPC C12N 9/22; C12N 15/11; C12Q 1/701; C07K 2319/85; C12N 2310/20; C12Q 2600/178

(57) ABSTRACT

Provided herein are engineered Cas13 proteins with enhanced collateral activity. The engineered proteins comprise RNA binding domains (RBDs) fusions within an active site-proximal loop within a higher eukaryotes and prokaryotes nucleotide-binding (HEPN) domain. Also provided are compositions comprising and methods of using the engineered Cas13 proteins to detect target nucleic acids at attomolar sensitivity.

### REFERENCE TO RELATED APPLICATIONS

The present application claims the priority benefit of U.S. provisional application No. 63/285,304, filed Dec. 2, 2021, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Nov. 11, 2022, is named RICEP0094WO_ST26.xml and is 218,830 bytes in size.

### BACKGROUND

### 1. Field

The present invention relates generally to the fields of medicine and diagnostics. More particularly, it concerns compositions and methods for the rapid and ultra-sensitive detection of nucleic acid molecules.

### 2. Description of Related Art

Cas13 orthologs, the single effectors of the Class 2 type VI CRISPR-Cas systems, are RNA-guided ribonucleases (Shmakov et al., 2015). The crRNAs of this family contain a direct repeat stem-loop that interacts with the Cas13 protein to form an RNase-inactive binary complex and a spacer sequence that base pairs with the target RNA (Konermann et al., 2018). The resulting Cas13:crRNA:target ternary complex undergoes a large-scale conformational change in which two higher eukaryotes and prokaryotes nucleotide-binding (HEPN) domains move toward each other to form a single catalytic pocket to cleave the target RNA. Intriguingly, the catalytic pocket localized on the outer surface of the target-activated Cas13 complex can non-specifically cleave any surrounding RNA molecules in a characteristic “collateral effect” (Liu et al., 2017; Zhang et al., 2018). This target-triggered collateral activity, originally an immune defense mechanism intended to induce host dormancy and prevent the propagation of invading phages, has been rapidly developed for in vitro nucleic acid detection (Broughton et al., 2020; Gootenberg et al., 2018). For example, the Cas13:crRNA surveillance complex is activated upon target recognition and performs collateral cleavage of nearby dye-quencher pairs linked by single-stranded RNA (ssRNA) (Gootenberg et al., 2017; Gootenberg et al., 2018) to rapidly generate measurable fluorescent or colorimetric signals (Gootenberg et al., 2017; Gootenberg et al., 2018; Qin et al., 2019).

The Leptotrichia wadei (Lwa) Cas13a has been widely used in nucleic acid-based diagnostics. Coupling LwaCas13a collateral activity to target preamplification, the Specific High-sensitivity Enzymatic Reporter unLOCKing (SHERLOCK) platform has detected attomolar levels of viral RNA and other endogenous RNA targets (Gootenberg et al., 2017). Combining LwaCas13a with auxiliary Csm6 endoribonuclease, SHERLOCKv2 shortened the detection time for a Dengue RNA target (Gootenberg et al., 2018). Integrating SHERLOCK detection with colorimetric barcoding technology, the CARMEN system achieved multiplexed detection of thousands of human virus samples (Ackerman et al., 2020). A LwaCas13a-based COVID-19 diagnostic test has undergone clinical validation (Patchsung et al., 2020) and received FDA Emergency Use Authorization during the pandemic (Kaminski et al., 2021). While LwaCas13a has a fluorescence-based detection sensitivity of ˜50 pM without target preamplification (Gootenberg et al., 2017), integrating electrochemical microfluidic chips with LwaCas13a can further improve the sensitivity for detecting short microRNA targets at concentrations as low as 2 pM (Bruch et al., 2021). LwaCas13a complexed with three crRNAs can detect SARS-CoV-2 targets at low femtomolar levels when coupled with a photolithography-fabricated microchamber array (Shinoda et al., 2021). However, attomolar sensitivity without target preamplification is critical for accurate and rapid clinical diagnostics, biological assessments, and environmental surveillance (Arnaout et al., 2021). Although Leptotrichia buccalis (Lbu) Cas13a was reported to have higher sensitivity for nucleic acid detection (Fozouni et al., 2021; Liu et al., 2021), it was promiscuous in the presence or absence of the target RNA in a spacer and reporter sequence-dependent manner (Gootenberg et al., 2018).

Despite the recent efforts to improve Cas13a-based nucleic acid detection (Gootenberg et al., 2017; Gootenberg et al., 2018; Qin et al., 2019; Ackerman et al. 2020; Patchsung et al., 2020; Kaminski et al., 2021; Bruch et al., 2021; Shinoda et al., 2021; Fozouni et al., 2021; Liu et al., 2021; Bruch et al., 2019; Son et al., 2021), engineering Cas13a proteins to have enhanced collateral activity has been challenging and it has not yet been successful, mostly due to the dynamic nature and the structural complexity of the RNase-active Cas13a enzymes.

### SUMMARY

Herein, a structural analysis of LwaCas13a was used to guide a novel protein engineering strategy designed to improve the collateral activity of LwaCas13a. Different types of RNA binding domains (RBDs) were fused to the tip of a unique β-hairpin loop proximal to the LwaCas13a active site in order to increase its RNA substrate binding affinity. Four of the seven RBD-LwaCas13a loop fusions enhanced the collateral activity and two of them (RBD #3L and RBD #4L) greatly improved the detection sensitivity for different targets, including SARS-CoV-2, Zika, Dengue, and Ebola virus RNA, and microRNAs. By coupling the collateral activity-enhanced LwaCas13a to an electrochemical sensor, ˜1 attomolar sensitivity and amplification-free detection was achieved for heat inactivated SARS-CoV-2 genomic RNA, indicating the potential of this approach for ultrasensitive detection of a wide range of RNA targets with clinical, biological, and environmental importance.

Provided herein are engineered Cas13 enzymes comprising a binding domain fusion, wherein the engineered Cas13 enzyme has enhanced collateral activity as compared to a Cas13 enzyme. The Cas13 enzyme may be a wild-type Cas13 enzyme and may have a sequence according to SEQ ID NO: 1. The engineered Cas13 enzyme may be derived from a Leptotrichia wadei (Lwa) Cas13a or Leptotrichia buccalis (Lbu) Cas13a.

The binding domain may be an RNA-binding domain, a DNA-binding domain, or an aptamer. The RNA-binding domain may be an RNA recognition motif, a double-stranded RNA binding domain, or a zinc-finger RNA binding domain. The RNA-binding domain may be adenosine deaminases acting on RNA 1 (ADAR1) double-stranded RNA binding motif 3 (DRBM3), heterogeneous nuclear ribonucleoprotein (hnRNP) A1 RNA recognition motif 1 (RRM1), heterogeneous nuclear ribonucleoprotein (hnRNP) A1 RNA recognition motif 2 (RRM2), hnRNP C RNA recognition motif (RRM), methyltransferase-like 3 (METTL3) zinc finger domain (ZnF), ADAR1 Z-DNA/RNA binding domain α (Zα), 30 or ADAR1 Z-DNA/RNA binding domain β (Zβ). The RNA-binding domain may have a sequence selected from the group consisting of: ADAR1⁷⁰⁸⁻⁸⁰¹ (amino acids 416-509 of SEQ ID NO: 2), hnRNPA1¹⁻¹⁰⁴ (amino acids 416-519 of SEQ ID NO: 3), hnRNPA1⁹⁸⁻¹⁹⁶ (amino acids 416-514 of SEQ ID NO: 4), hnRNPC²⁻¹⁰⁶ (amino acids 416-520 of SEQ ID NO: 5), METTL3²⁵⁹⁻³⁵⁷ (amino acids 416-514 of SEQ ID NO: 6), ADAR1¹²⁶⁻²⁰² (amino acids 416-492 of SEQ ID NO: 7), and ADAR1²⁹³⁻³⁶⁶ (amino acids 416-489 of SEQ ID NO: 8). The RNA-binding domain may be the heterogeneous nuclear ribonucleoprotein (hnRNP) A1 RNA recognition motif 1 (RRM1) or the hnRNP C RNA recognition motif (RRM).

The binding domain may be fused to the N-terminus or the C-terminus of the Cas13 enzyme. A linker (e.g., a flexible linker) may or may not be positioned between the binding domain and the Cas13 enzyme. The binding domain may be an RNA binding domain selected from the group consisting of an hnRNP C RNA recognition motif (RRM) and an ADAR1 Z-DNA/RNA binding domain α (Zα). The engineered protein may have a sequence at least 90%, 91%, 92%, 93%, 94%, 95%, 96%, 97%, 98%, or 99% identical to SEQ ID NO: 22 or SEQ ID NO: 24. The engineered protein may have a sequence of SEQ ID NO: 22 or 24.

The binding domain may be fused to the Cas13 enzyme within a structural loop of the Cas13 enzyme. The structural loop may be proximal to the enzyme's active site. The structural loop may be a beta hairpin loop. The loop may be within a higher eukaryotes and prokaryotes nucleotide-binding (HEPN) domain of the Cas13 enzyme. In some aspects, the fusion protein does not comprise any linkers. The fusion protein may further comprise a linker positioned between the N-terminal end of the binding domain and the Cas13 enzyme and/or between the C-terminal end of the binding domain and the Cas13 enzyme.

The Cas13 enzyme may be Leptotrichia wadei (Lwa) Cas13a and the structural loop may be a beta hairpin loop in the HEPNI domain. The beta hairpin loop in the HEPN1 domain may correspond to amino acids G410-G425 of SEQ ID NO: 1. The binding domain may be inserted into the Cas13 enzyme between amino acids corresponding to N415 and N416 of SEQ ID NO: 1, between amino acids corresponding to N416 and K417 of SEQ ID NO: 1, or between amino acids corresponding to K417 and G418 of SEQ ID NO: 1.

The binding domain may be an RNA-binding domain selected from the group consisting of adenosine deaminases acting on RNA 1 (ADAR1) double-stranded RNA binding motif 3 (DRBM3), heterogeneous nuclear ribonucleoprotein (hnRNP) A1 RNA recognition motif 1 (RRM1), heterogeneous nuclear ribonucleoprotein (hnRNP) A1 RNA recognition motif 2 (RRM2), hnRNP C RNA recognition motif (RRM), and ADAR1 Z-DNA/RNA binding domain α (Zα). The binding domain may be an RNA-binding domain selected from the group consisting of heterogeneous nuclear ribonucleoprotein (hnRNP) A1 RNA recognition motif 2 (RRM2) and hnRNP C RNA recognition motif (RRM). The engineered protein may have a sequence at least 90%, 91%, 92%, 93%, 94%, 95%, 96%, 97%, 98%, or 99% identical to SEQ ID NO: 4 or SEQ ID NO: 5. The engineered protein may have a sequence of SEQ ID NO: 4 or 5.

The engineered protein may have a sequence at least 90%, at least 91%, at least 92%, at least 93%, at least 94%, at least 95%, at least 96%, at least 97%, at least 98%, at least 99% or 100% identical to SEQ ID NO: 2 (Loop 1 N415/N416 fusion, amino acids 416-509 are the ADAR1 DRBM3 insertion), SEQ ID NO: 3 (Loop 1 N415/N416 fusion, amino acids 416-519 are the hnRNP A1 RRM1 insertion), SEQ ID NO: 4 (Loop 1 N415/N416 fusion, amino acids 416-514 are the hnRNP A1 RRM2 insertion), SEQ ID NO: 5 (Loop 1 N415/N416 fusion, amino acids 416-520 are the hnRNP C RRM insertion), SEQ ID NO: 6 (Loop 1 N415/N416 fusion, amino acids 416-514 are the METTL3 ZnF insertion), SEQ ID NO: 7 (Loop 1 N415/N416 fusion, amino acids 416-492 are the ADAR1 Zα insertion), SEQ ID NO: 8 (Loop 1 N415/N416 fusion, amino acids 416-489 are the ADAR1 Zβ insertion), SEQ ID NO: 9 (Loop 1 N416/K417 fusion, amino acids 417-515 are the hnRNP A1 RRM2insertion), SEQ ID NO: 10 (Loop 1 N416/K417 fusion, amino acids 417-521 are the hnRNP C RRM insertion), SEQ ID NO: 11 (Loop 1 K417/G418 fusion, amino acids 418-516 are the hnRNP A1 RRM2 insertion), SEQ ID NO: 12 (Loop 1 K417/G418 fusion, amino acids 418-522 are the hnRNP C RRM insertion), SEQ ID NO: 13 (Loop 2 S996/K997 fusion, amino acids 997-1095 are the hnRNP A1 RRM2 insertion), SEQ ID NO: 14 (Loop 2 S996/K997 fusion, amino acids 997-1101 are the hnRNP C RRM insertion), SEQ ID NO: 15 (Loop 2 S996/K997 fusion with short linkers, amino acids 997-1000 are linker 1, amino acids 1001-1099 are the hnRNP A1 RRM2 insertion, and amino acids 1100-1103 are linker 2), SEQ ID NO: 16 (Loop 2 S996/K997 fusion with short linkers, amino acids 997-1000 are linker 1, amino acids 1001-1105 are the hnRNP C RRM insertion, and amino acids 1106-1109 are linker 2), SEQ ID NO: 17 (Loop 2 S996/K997 fusion with long linkers, amino acids 997-1011 are linker 1, amino acids 1012-1110 are the hnRNP A1 RRM2 insertion, and amino acids 1111-1125 are linker 2), SEQ ID NO: 18 (Loop 2 S996/K997 fusion with long linkers, amino acids 997-1011 are linker 1, amino acids 1012-1116 are the hnRNP C RRM insertion, and amino acids 1117-1131 are linker 2), SEQ ID NO: 19 (N-terminal fusion, amino acids 1-94 are the ADAR1 DRBM3 insertion, amino acids 95-126 are a linker), SEQ ID NO: 20 (N-terminal fusion, amino acids 1-104 are the hnRNP A1 RRM1 insertion, amino acids 105-136 are a linker), SEQ ID NO: 21 (N-terminal fusion, amino acids 1-99 are the hnRNP A1 RRM2 insertion, amino acids 100-131 are a linker), SEQ ID NO: 22 (N-terminal fusion, amino acids 1-105 are the hnRNP C RRM insertion, amino acids 106-137 are a linker), SEQ ID NO: 23 (N-terminal fusion, amino acids 1-99 are the METTL3 ZnF insertion, amino acids 100-131 are a linker), SEQ ID NO: 24 (N-terminal fusion, amino acids 1-77 are the ADAR1 Zα insertion, amino acids 78-109 are a linker), SEQ ID NO: 25 (N-terminal fusion, amino acids 1-74 are the ADAR1 Zβ insertion, amino acids 75-106 are a linker), SEQ ID NO: 26 (C-terminal fusion, amino acids 1153-1184 are a linker, amino acids 1185-1278 are the ADAR1 DRBM3 insertion), SEQ ID NO: 27 (C-terminal fusion, amino acids 1153-1184 are a linker, amino acids 1185-1288 are the hnRNP A1 RRM1 insertion), SEQ ID NO: 28 (C-terminal fusion, amino acids 1153-1184 are a linker, amino acids 1185-1283 are the hnRNP A1 RRM2 insertion), SEQ ID NO: 29 (C-terminal fusion, amino acids 1153-1184 are a linker, amino acids 1185-1289 are the hnRNP C RRM insertion), SEQ ID NO: 30 (C-terminal fusion, amino acids 1153-1184 are a linker, amino acids 1185-1283 are the METTL3 ZnF insertion), SEQ ID NO: 31 (C-terminal fusion, amino acids 1153-1184 are a linker, amino acids 1185-1261 are the ADAR1 Zα insertion), SEQ ID NO: 32 (C-terminal fusion, amino acids 1153-1184 are a linker, amino acids 1185-1258 are the ADAR1 Zβ insertion), SEQ ID NO: 121 (Loop 1 N415/N416 fusion, amino acids 416-514 are the hnRNP A1 RRM1 insertion, amino acids 515-524 is a linker, and amino acids 525-629 are the hnRNP A1 RRM2 insertion), or SEQ ID NO: 122 (Loop 1 N415/N416 fusion, amino acids 416-520 are the hnRNP A1 RRM2 insertion, amino acids 521-530 is a linker, and amino acids 531-629 are the hnRNP A1 RRM1 insertion).

Provided herein are methods of enhancing the collateral activity of a Cas13 protein, the method comprising fusing a binding domain to the Cas13 protein, testing the collateral activity of the fusion protein, and identifying the fusion protein as having enhanced collateral activity if its collateral activity is increased as compared to a Cas13 enzyme. The Cas13 enzyme may be a wild-type Cas13 enzyme and may have a sequence according to SEQ ID NO: 1.

The binding domain may be an RNA-binding domain, a DNA-binding domain, or an aptamer. The RNA-binding domain may be an RNA recognition motif, a double-stranded RNA binding domain, or a zinc-finger RNA binding domain. The RNA-binding domain may be adenosine deaminases acting on RNA 1 (ADAR1) double-stranded RNA binding motif 3 (DRBM3), heterogeneous nuclear ribonucleoprotein (hnRNP) A1 RNA recognition motif 1 (RRM1), heterogeneous nuclear ribonucleoprotein (hnRNP) A1 RNA recognition motif 2 (RRM2), hnRNP C RNA recognition motif (RRM), methyltransferase-like 3 (METTL3) zinc finger domain (ZnF), ADAR1 Z-DNA/RNA binding domain α (Zα), or ADAR1 Z-DNA/RNA binding domain β (Zβ). The RNA-binding domain may have a sequence selected from the group consisting of: ADAR1⁷⁰⁸⁻⁸⁰¹ (amino acids 416-509 of SEQ ID NO: 2), hnRNPA1¹⁻¹⁰⁴ (amino acids 416-519 of SEQ ID NO: 3), hnRNPA1⁹⁸⁻¹⁹⁶ (amino acids 416-514 of SEQ ID NO: 4), hnRNPC²⁻¹⁰⁶ (amino acids 416-520 of SEQ ID NO: 5), METTL3²⁵⁹⁻³⁵⁷ (amino acids 416-514 of SEQ ID NO: 6), ADAR1¹²⁶⁻²⁰² (amino acids 416-492 of SEQ ID NO: 7), and ADAR1²⁹³⁻³⁶⁶ (amino acids 416-489 of SEQ ID NO: 8). The RNA-binding domain may be the heterogeneous nuclear ribonucleoprotein (hnRNP) A1 RNA recognition motif 1 (RRM1) or the hnRNP C RNA recognition motif (RRM).

The binding domain may be fused to the N-terminus or the C-terminus of the Cas13 enzyme. A linker (e.g., a flexible linker) may or may not be positioned between the binding domain and the Cas13 enzyme. The binding domain may be an RNA binding domain selected from the group consisting of an hnRNP C RNA recognition motif (RRM) and an ADAR1 Z-DNA/RNA binding domain α (Zα).

The binding domain may be fused to the Cas13 enzyme within a structural

loop of the Cas13 enzyme. The structural loop may be proximal to the enzyme's active site. The structural loop may be a beta hairpin loop. The loop may be within a higher eukaryotes and prokaryotes nucleotide-binding (HEPN) domain of the Cas13 enzyme. In some aspects, the fusion protein does not comprise any linkers. The fusion protein may further comprise a linker positioned between the N-terminal end of the binding domain and the Cas13 enzyme and/or between the C-terminal end of the binding domain and the Cas13 enzyme.

The Cas13 enzyme may be Leptotrichia wadei (Lwa) Cas13a and the structural loop may be a beta hairpin loop in the HEPN1 domain. The beta hairpin loop in the HEPN1 domain may correspond to amino acids G410-G425 of SEQ ID NO: 1. The binding domain may be inserted into the Cas13 enzyme between amino acids corresponding to N415 and N416 of SEQ ID NO: 1, between amino acids corresponding to N416 and K417 of SEQ ID NO: 1, or between amino acids corresponding to K417 and G418 of SEQ ID NO: 1.

The binding domain may be an RNA-binding domain selected from the group consisting of adenosine deaminases acting on RNA 1 (ADAR1) double-stranded RNA binding motif 3 (DRBM3), heterogeneous nuclear ribonucleoprotein (hnRNP) A1 RNA recognition motif 1 (RRM1), heterogeneous nuclear ribonucleoprotein (hnRNP) A1 RNA recognition motif 2 (RRM2), hnRNP C RNA recognition motif (RRM), and ADAR1 Z-DNA/RNA binding domain α (Zα). The binding domain may be an RNA-binding domain selected from the group consisting of heterogeneous nuclear ribonucleoprotein (hnRNP) A1 RNA recognition motif 2 (RRM2) and hnRNP C RNA recognition motif (RRM).

Provided herein are fusion proteins made by the methods of the present embodiments.

Provided herein are compositions comprising (i) the engineered Cas13 enzymes or the fusion proteins provided herein and (ii) a crRNA. The crRNA may comprise a guide sequence that is capable of hybridizing to a target RNA sequence. The compositions may further comprise a reporter RNA. The reporter RNA may comprise at least 11 nucleotides. The reporter RNA may comprise 11, 12, 13, 14, 15, 16, 17, 18, 19, or 20 nucleotides. The reporter RNA may comprise a poly-uridine sequence. The report RNA may comprise a detectable label. The reporter RNA may comprise a fluorescent molecule and a quencher molecule. The fluorescent molecule may be carboxyfluorescein and the quencher molecule may be Iowa Black. The reporter RNA comprises a redox active molecule. The composition may further comprise at least one salt. The composition may further comprise Mg²⁺. The composition may be lyophilized.

Provided herein are methods of detecting the presence of a target nucleic acid in a sample, the methods comprising contacting the sample with the compositions provided herein and detecting a signal from the detectable label. The sample may be a biological sample or environmental sample. The sample may comprise saliva, urine, plasma, or serum. The sample may comprise extracted nucleic acids or amplified nucleic acids. The sample may be a crude sample. The sample may be a non-extracted sample.

The target nucleic acid may have not been amplified prior to performing the method. The target nucleic acid may be a viral nucleic acid. The target nucleic acid may be a viral RNA. The target nucleic acid may be a SARS-CoV-2 RNA.

The target nucleic acid may be a SARS-CoV-2 N gene. The crRNA may have a sequence of SEQ ID NO: 56.

The target nucleic acid may be a SARS-CoV-2 RdRp gene. The crRNA may have a sequence of SEQ ID NO: 57.

The target nucleic acid may be a Zika virus POLY gene. The crRNA may have a sequence of SEQ ID NO: 70.

The target nucleic acid may be a Dengue virus POLY gene. The crRNA may have a sequence of SEQ ID NO: 71.

The target nucleic acid may be an Ebola virus L gene. The crRNA may have a sequence of SEQ ID NO: 72.

The target nucleic acid may be a miRNA. The target nucleic acid may be hsa-miR-19b. The crRNA may have a sequence of SEQ ID NO: 73. The target nucleic acid may be hsa-miR-2392. The crRNA may have a sequence of SEQ ID NO: 74.

The method may be performed in a buffer comprising 20-100 mM Tris pH 7.0-8.0 and Mg²⁺.

The method may be coupled with an electrochemical fluid chip. The method may be coupled with a graphene field-effect transistor (gFET) biosensor.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Design of N-and C-terminal RNA binding domain (RBD) fusions to LwaCas13a. (FIG. 1A) Seven RNA binding domains (RBDs) were selected and fused to LwaCas13a at N- or C-terminus with a flexible XTEN linker (32 aa). (FIGS. 1B and 1C) Background-subtracted fluorescence comparing the collateral activity between WT and RBD #1N-#7N, and between WT and RBD #1C-#7C. For FIGS. 1B and C, 10 pM of synthetic SARS-CoV-2 N gene fragment target (SEQ ID NO: 33; T1) were detected by WT and its N or C fusion variants. The background-subtracted fluorescence (arbitrary units, AU) was calculated by taking the raw fluorescence signal of the testing sample and subtracting that of the corresponding no-target control, in which the target RNA is replaced by nuclease-free water. Each bar was plotted from four technical replicates with standard error of means (s.e.m.) at an assay time of 30 min. Two-tailed P values were calculated using unpaired t-tests: NS, not significant. *P=0.04; **P=0.0022; ***P=0.0008 for RBD #7C, P=0.0002 for both RBD #4 C and #5 C; and ****P<0.0001; compared to WT.

FIGS. 2A-G. Structure-guided design of collateral activity-enhanced RBD-LwaCas13a fusion proteins. (FIG. 2A) Domain organization of LwaCas13a by alignment to LbuCas13a (PDB: 5XWP). Catalytic motifs are R474-H479 in HEPN1 and R1046-H1051 in HEPN2. Loop regions for RBD insertion are G410-G425 (denoted as Loop 1) and N992-G1004 (denoted as Loop 2). NTD, N-terminal domain. (FIGS. 2B) Ribbon representation of the predicted structure of LwaCas13a:crRNA:target RNA ternary complex. The two loops and active site are shown in the zoomed-in panel (FIG. 2C). (FIG. 2C) Zoomed-in view of catalytic residues and two active site-proximal loops. Loop-fusion proteins were constructed by inserting RBDs between indicated residues, including N415, N416, K417, and G418, or S996 and K997. (FIG. 2D) Background-subtracted fluorescence comparing the collateral activity between WT and seven Loop 1 fusions (RBD #1L-#7L, in which RBDs were inserted between N415 and N416 in Loop 1). (FIG. 2E and 2F) Surface views of RBD #3, i.e., hnRNP A1 RRM2 (PDB: 5MPL), and RBD #4, i.e., hnRNP C RRM (PDB: 2MXY). N- and C-termini, and RNA binding regions for both RBDs are labeled. (FIG. 2G) Background-subtracted fluorescence showing the collateral activity of WT, and RBD #3 and RBD #4 loop-fusion variants. Six constructs for each RBD were tested, including three Loop 1 variants with RBD directly inserted between N415 and N416, between N416 and K417, or between N417 and G418, and three Loop 2 variants with RBD inserted between S996 and K997, including direct insertion with no linker, with flanking GSSG linker (SEQ ID NO: 123), or (GGGGS); linker (SEQ ID NO: 124), respectively. For FIGS. 2D and 2G, 10 pM of synthetic RNA (SEQ ID NO: 33, T1) was detected by WT and loop-fusion variants. Bar plot represented the 30-min background-subtracted fluorescence expressed as mean±s.e.m. from four technical replicates. Two-tailed p values were calculated using unpaired t-tests: *P=0.0177; **P=0.0037, ***P=0.0006, ****P<0.0001.

FIGS. 3A-D. Optimizing reporter length for enhanced collateral activity. (FIG. 3A) Representative gel of the cleavage of 5′-FAM-labeled U₅, U₁₁, U₁₅, and U₂₀ reporters by WT LwaCas13a and RBD #3L after a 10-min incubation. (FIG. 3B) Quantified percentage of cleaved products as shown in (FIG. 3A), represented by mean±s.e.m. of six replicates from two independent experiments each with three technical replicates for WT and RBD #3L, and of three technical replicates for RBD #4L. Gels were analyzed by ImageJ. The percentage of cleaved products was calculated by the intensity of product bands divided by the summed intensity of the product and substrate bands within one lane and normalized to the reporter-only control. Two-tailed P values were calculated using unpaired t-tests with Welch's correction: *P=0.0116; ***P=0.0004; ****P<0.0001; compared to WT. For each group of three bars, the left bar represents RBD #3L, the center bar represents RBD #4L, and the right bar represents WT. (FIG. 3C) Time course of raw fluorescence generated by cleavage of RNaseAlert (IDT), fluorophore-quencher pairs connected by U₅ or U₁₁. Reporters were incubated with WT, RBD #3L, or #4L with the presence or absence of 10 pM of the TI (SEQ ID NO: 33) target. Controls were reactions in the absence of a target (con-RBD #3L, con-RBD #4L, and con-WT). Data are represented as mean±s.e.m. The error bar is shown from four technical replicates. (FIG. 3D) Mean slope±95% confidence interval was calculated from panel (FIG. 3C) fluorescence signal over 30 min for U₅ and RNaseAlert, and over 10 min for U₁₁ by simple linear regression. Adjusted P values were calculated using two-way ANOVA with Dunnett's test. **P=0.011; ****P<0.0001; compared to WT. For each group of three bars, the left bar represents RBD #3L, the center bar represents RBD #4L, and the right bar represents WT.

FIGS. 4A-C. RBD #3L and #4L enhance the collateral activity in a spacer-independent manner and maintain the specificity for target recognition. (FIG. 4A) Background-subtracted fluorescence over 120 min comparing the collateral activity of WT, RBD #3L, and RBD #4L with 11 crRNAs (cr4-cr14) evenly tiled across ssRNA1 target (T3). Data are represented as mean±s.e.m. from four technical replicates. (FIG. 4B) Background-subtracted fluorescence early time point (30 min) from panel (FIG. 4A), showing increased collateral cleavage by RBD #3L and #4L. (FIG. 4C) Single-point mutations (MM2, MM6, MM10, MM14, MM18, MM22, and MM26, left panel) and double-point mutations (DM1, DM5, DM9, DM13, DM17, DM21 and DM25, right panel) were introduced to the 28-nt RNA target ([SEQ ID NOS: 37-50 and 126-132], T5-T25). For each group of three bars, the top bar represents RBD #3L, the center bar represents RBD #4L, and the bottom bar represents WT. Bar graphs are meant s.e.m. from four technical replicates, indicating the normalized fold change of background-subtracted fluorescence of point mutations to the corresponding perfectly matched (PM) target ([SEQ ID NO: 36], T4). Adjusted P values were calculated using multiple Holm-Sidak t-tests. Unless indicated, no significant difference was observed from each point mutations, i.e., RBD #3L versus WT, and i.e., RBD #4L versus WT. *p=0.03498, compared to WT LwaCas13a.

FIGS. 5A-F. Improved detection of various spiked-in targets. Background-subtracted fluorescence in the detection of 10 pM of: (FIG. 5A) SARS-CoV-2 N gene synthetic targets ([SEQ ID NO: 33], T1) spiked into 8% VTM, 8% saliva, or reaction buffer; (FIG. 5B) Zika synthetic targets ([SEQ ID NO: 51], T27) spiked into 8% urine, or reaction buffer; (FIG. 5C) Dengue synthetic targets ([SEQ ID NO: 52], T28) spiked into 2.5% serum or reaction buffer; (FIG. 5D) Ebola ([SEQ ID NO: 53], T29) synthetic targets spiked into 2.5% plasma, or reaction buffer; (FIG. 5E) miR-19b ([SEQ ID NO: 54], T30) spiked into 2.5% serum, 2.5% plasma, or reaction buffer; and (FIG. 5F) 200 pM of miR-2392 ([SEQ ID NO: 55], T31) spiked into 2.5% serum, 8% urine, or reaction buffer. All reactions were supplemented with 50 ng total RNA extracted from HEK293T cells. Bar graphs are the 30-min background-subtracted fluorescence mean±s.e.m. from four technical replicates. For each group of three bars, the left bar represents RBD #3L, the center bar represents RBD #4L, and the right bar represents WT. Two-tailed P values were calculated using unpaired t-tests: *P<0.05; **P<0.01; ***P<0.001; and ****P<0.0001; compared to the corresponding WT under the same testing conditions. More specifically: FIG. 5A, ***P=0.0001; FIG. 5B, ***P=0.0002 for RBD #3L and 0.0005 for RBD #4L; FIG. 5E, *P=0.0140; FIG. 5F, ***P=0.001, **P=0.0045.

FIGS. 6A-J. RBD fusions facilitate electrochemical detection of RNA at attomolar sensitivity. (FIGS. 6A-C) Electrochemical detection of synthetic RNA target. WT (FIG. 6A), RBD #3L (FIG. 6B), or RBD #4L (FIG. 6C) RNPs were activated with various concentrations of the synthetic SARS-CoV-2 N gene target ([SEQ ID NO: 33], T1). Blank samples are RNPs without the target. (FIGS. 6D-F) Electrochemical detection of inactive virus RNA. WT (FIG. 6D), RBD #3L (FIG. 6E), or RBD #4L (FIG. 6F) RNPs were activated by serial dilution of the inactive SARS-CoV-2 viral genome. Blank samples are RNPs without the target (n=6 for the 0 aM sample from FIG. 6D, 0, 10⁰ and 10¹ aM from FIG. 6E, and 10² aM from FIG. 6F; n=9 for 10² aM from FIG. 6E). (FIGS. 6G-J) Electrochemical detection of SARS-CoV-2 RNA from unextracted raw clinical VTM samples. VTM were leftovers for nasopharyngeal swab storage, and the viral load was quantified by RT-qPCR with standard curve analysis using purified RNA. WT (FIG. 6G) was tested with three positive samples (P2, P4, and P8); RBD #3L (FIG. 6H) was tested with five negative samples (N1-N5) and 11 positive samples (P1-P11); and RBD #4L (FIG. 6I-J) was tested with five negative samples (N6-N10) and 11 positive samples (P1-P11). Clinical samples were subjected to extraction-free 5-min quick treatment before adding to the reactions. Blank samples were tested in parallel with clinical samples and used VTM as input. (FIGS. 6H and 6I), Box plots with the 25^{th} and the 75^{th} percentile boundaries and the whiskers indicating the minimum, maximum, and mean values of electrochemical signal change (ΔI %) of clinical samples each from three technical replicates. Dotted lines indicate the threshold for electrochemical sensor detection and were set to mean+2×s.d. of the blank. Concordance tables between RBD #3L/RBD #4L-sensor and RT-qPCR for clinical samples are shown. (FIG. 6J) Quantification of positive clinical samples (Mean±s.d. from three replicates) using RT-qPCR with purified and concentrated RNA as input. For FIGS. 6A-G, bar plots represented mean±s.d. of the ΔI % from three technical replicates unless otherwise indicated. Two-tailed P values were calculated using unpaired t-tests with Welch's correction. Unless indicated, no significant change was observed compared to the corresponding no-target blank sample. *P<0.05, **P<0.01, ***P<0.001, ****P<0.0001. More specifically: FIG. 6A, *P=0.0109, **P=0.0027, ***P=0.0008; FIG. 6B, *P=0.0139 for 10¹ and *P=0.0103for 5×10¹ aM target samples, **P=0.0048, ***P=0.0002 and 0.0003 for 10⁴ and 10⁵ aM target samples; FIG. 6C, *P=0.0366, **P=0.0081 and 0.0015 for 5×10¹ and 10² aM target samples, ***P=0.0001, 0.0002 and 0.0009 for 10⁴, 10⁵, and 10⁶ aM target samples; FIG. 6E, *P=0.0343, **P=0.0053, ***P=0.0002 for both 10¹ and 10⁴ aM target samples; FIG. 6F, *P=0.0274, **P=0.0073, ***P=0.0004.

FIGS. 7A-C. Candidate RBDs and testing of N-and C-terminal fusions. (FIG. 7A) Seven RNA binding domains (RBDs) were selected and tethered to N- or C-terminus ends of LwaCas13a via a flexible XTEN linker (32 aa). The PDB ID, Uniprot accession number (with amino acid residue positions in the original protein), and length of the domain are listed. (FIGS. 7B-C) Time course of N- and C-terminal RBD fusions. Background-subtracted fluorescence in the detection of 10 pM synthetic SARS-CoV-2 N gene fragment target ([SEQ ID NO: 33], T1) using N- (FIG. 7B) and C- (FIG. 7C) terminal RBD fusions, expressed as mean±s.e.m. from four technical replicates.

FIGS. 8A-H. Testing of RBD loop-fusion variants. Each of the fusion proteins was assembled with the crRNA as described in Methods. (FIGS. 8A-C) Testing of seven candidate RBDs (RBD #1-RBD #7) fused to LwaCas13a. (FIGS. 8A and 8B) Raw fluorescence over a course of 120 min in the reactions with or without 10 pM synthetic RNA targets: FIG. 8A, SARS-CoV-2 RdRp gene fragment ([SEQ ID NO: 34], T2); FIG. 8B, ssRNA1 ([SEQ ID NO: 35], T3). NTC, non-target control. (FIG. 8C) The 120-min background-subtracted fluorescence from FIGS. 8A and 8B. (FIGS. 8D-F) Testing of RBD #3 and RBD #4 fused to various positions on Loop 1 of LwaCas13a. The six variants were RBD #3 and #4 inserted after N415, N416, and K417, respectively. (FIGS. 8D and 8E) Raw fluorescence data over a course of 120 min in the reactions with or without 10 pM synthetic RNA targets. FIG. 8D, SARS-CoV-2 RdRp gene fragment ([SEQ ID NO: 34], T2); FIG. 8E, ssRNA1 ([SEQ ID NO: 35], T3). (FIG. 8F) The 30-min background-subtracted fluorescence from FIGS. 8D and 8E. (FIGS. 8G and 8H) Testing of tandem RBD insertions. The four variants for comparison were RBD #3, RBD #4, RBD #3-linker-RBD #4, and RBD #4-linker-RBD #3 inserted after N415 on Loop 1 of LwaCas13a, respectively. A 10-aa flexible linker (SGGSGGSGGS; SEQ ID NO: 125) was used to connect two RBDs in tandem. (FIG. 8G) Background-subtracted fluorescence data over a course of 60 min in the reactions with 10 PM SARS-CoV-2 N gene fragment synthetic RNA targets ([SEQ ID NO: 33], T1). (FIG. 8H) The 30-min and 60-min background-subtracted fluorescence from FIG. 8F. For each group of five bars, they represent, from left to right, RBD #3L, RBD #4L, RBD #3 #4L (RBD3-linker-RBD4), RBD #4 #3L (RBD4-linker-RBD3), and WT. A 10-aa f flexible linker (SGGSGGSGGS; SEQ ID NO: 125) was used to connect two RBDs in tandem. (FIGS. 8A, 8B, 8D, 8E, and 8G) Lines and error bars represented mean±s.e.m. from four technical replicates: (FIGS. 8C, 8F, and 8H) bar plots were expressed as mean±s.e.m. from four technical replicates. Two-tailed p values were calculated using unpaired t-tests with Welch's correction: ns, not significant, *p<0.05, **p<0.01, ***p<0.001, ****p<0.0001.

FIGS. 9A-E. Optimizing reporter length for enhanced collateral activity. (FIGS. 9A-B) Representative gel images of the cleavage of 5′-FAM labeled U₅, U₁₁, U₁₅ and U₂₀ reporters by WT LwaCas13a and RBD #3L over 0-30 min. RBD fusions and WT were tested side-by-side. (FIGS. 10C-E) Quantified percentage of cleaved products as shown in (FIGS. 9A-B), represented by mean±s.e.m. of three technical replicates from two independent experiments. Gels were analyzed by ImageJ. The percentage of cleaved products was calculated by the intensity of product bands divided by the summed intensity of the product and substrate bands within one lane and normalized to the reporter-only control.

FIGS. 10A-F. LoD analysis of RBD #3L, RBD #4L, and WT LwaCas13a in reactions supplemented with BSA and Triton X-100. (FIGS. 10A-C) Raw fluorescence data measured for 120 min, expressed as mean±s.e.m. from four technical replicates. RBD #3L, RBD #4L and WT LwaCas13a were complexed with crRNA (cr1) and tested with 2.5, 1, 0.25, 0.1, 0.025 pM of the synthetic RNA ([SEQ ID NO: 33], T1) in reaction buffer supplemented with 100 μg/mL BSA and 0.01% Triton X-100 (as in FIG. 17B). The 0 PM target samples were used as non-target control (NTC). (FIG. 10D) The bar plot represented the 120-min background-subtracted fluorescence expressed as mean±s.e.m. from four technical replicates. Adjusted p values were calculated using two-way ANOVA with Dunnett's test: **p=0.0059, ***p=0.0007, ****p<0.0001, compared to background signal (non-target control). (FIG. 10E) The velocities of each reaction within 60 min were obtained by linear regression from (FIGS. 10A-C) and were plotted as functions of the target concentration. Outliers were identified by the ROUT method with a Q=5%. (FIG. 10F) Analytical LoD calculated using the equation: LoD=3·SD_{NTC}/Slope. SD_{NTC} was the standard deviation of the reaction velocity from the no-target control samples. The slope was determined by linear regression of the reaction velocity against target concentration from FIG. 10E.

FIGS. 11A-G. Quantification of RNA binding with RBD #3L, RBD #4L, and WT LwaCas13a. (FIGS. 11A and 11C) Representative EMSA gel images of inactive HEPN2 mutants (R1046A/H1051A), that is, dRBD #3L, dRBD #4L, and dWT complexed with crRNA binding to 5 nM of body-radiolabeled (32P) target RNA. (FIG. 11A, [SEQ ID NO: 133], T26; FIG. 11C, [SEQ ID NO: 33], T1). The protein: crRNA complex with a molar ratio of 1:0.95 was serially diluted to 288, 192, 128, 86, 57, 38, 25, 17, 12, 8 and 5 nM. The 0-nM control was target RNA with reaction buffer. (FIGS. 11B and 11D) Calculation of binding affinity between target RNA and RNP. Bound and unbound fractions from FIGS. 11A and 11C were quantified by densitometry and fitted to standard binding isoforms. Mean from two technical replicates was plotted. (FIG. 11E) Representative EMSA gel images of protein:crRNA:target ternary complex binding with 100 nM of 5′-FAM-labeled U₂₀ reporter. The dRBD #3L, dRBD #4L and dWT complex with crRNA with a molar ratio of 2:1 was serially diluted to 2, 1.8, 1.6, 1.4, 1.2, 1, 0.8, 0.4, 0.2, 0.1 and 0.05 μM. An equal amount of 50 pM non-labeled target ([SEQ ID NO: 33], T1) was added to the RNPs to mimic the target-bound ternary complex. The 0-μM control was the reporter with reaction buffer. (FIG. 11F) Calculation of binding affinity between reporter RNA and target-bound RNP. Bound and unbound fractions from FIG. 11E were quantified by densitometry and fitted to standard binding isoforms. Mean from two technical replicates was plotted. (FIG. 11G) Dissociation constant (KD) for target and reporter RNA binding with RBD #3L, RBD #4L and the WT. Means with 95% CI were listed.

FIGS. 12A-E. Kinetic analysis of RBD #3L, RBD #4L and WT LwaCas13a. (FIGS. 12A-C) Progress curves of cleaved reporters versus time. Data were plotted as mean±s.e.m. from two independent experiments each with four technical replicates. The RNP complex of RBD #3L, RBD #4L, and WT was incubated with 50 PM of the targets ([SEQ ID NO: 33], T1) for 10 min at 37° C. prior to cleavage reaction using 6400, 3200, 1600, 800, 400, 200, 100 and 50 nM of the U₁₁ substrates. The initial velocities (within 600 s) of each reaction were obtained by linear regression. (FIG. 12D) Initial velocities from two independent experiments each with four technical replicates obtained from FIG. 12A-C were plotted as mean±s.e.m. versus the U₁₁ reporter concentration and fitted to a Michaelis-Menten curve. (FIG. 12E) Summary of Michaelis-Menten kinetic parameters (mean with 95% CI) in reactions using RBD #3L, RBD #4L and the WT.

FIGS. 13A-J. Testing RBD #3L, #4L and WT LwaCas13a in the detection of viral RNA and microRNA spiked-in samples. Background-subtracted fluorescence over 120 min in the detection of: FIGS. 13A and 13B, 10 pM of SARS-CoV-2 N gene synthetic targets spiked into 8% VTM, 8% saliva, or reaction buffer; FIG. 13C, 10 pM of Zika synthetic targets spiked into 8% urine, or reaction buffer; FIG. 13D, 10 pM of Dengue synthetic targets spiked into 2.5% serum, or reaction buffer; FIG. 13E, 10 pM of Ebola synthetic targets spiked into 2.5% plasma, or reaction buffer; FIGS. 13F-G, 10 pM of miR-19b spiked into 2.5% plasma, 2.5% serum, or reaction buffer; FIGS. 13H-I, 200 pM of miR-2392 spiked into 2.5% serum, 8% urine, or reaction buffer; FIG. 13J, 10 pM of miR-2392 in reaction buffer. All reactions were supplemented with 50 ng total RNA extracted from HEK293T cells but FIG. 13J. Background-subtracted fluorescence data were expressed as mean±s.e.m. from four technical replicates (FIGS. 13A-I), or 2 independent experiments, each with four technical replicates (FIG. 13J).

FIGS. 14A-D. Testing RBD #3L and WT LwaCas13a in the detection of viral RNA spiked-in samples. (Related to FIG. 5). Background-subtracted fluorescence over 120 min in the detection of: FIG. 14A, 50 PM SARS-CoV-2 N gene synthetic target ([SEQ ID NO: 33], T1) spiked into 16% VTM, 8% VTM, or reaction buffer; FIG. 14B, 50 pM of Zika synthetic target ([SEQ ID NO: 51], T27) spiked into 16% urine, 8% urine, or reaction buffer; FIG. 14C, 50 pM of Dengue synthetic target ([SEQ ID NO: 52], T28) spiked into 4.8% serum, 2.5% serum, or reaction buffer; FIG. 14D, 50 pM of Ebola synthetic target ([SEQ ID NO: 53], T29) spiked into 4.8% plasma, 2.5% plasma or reaction buffer. All reactions were supplemented with 5 ng/μL total RNA extracted from HEK293T cells as background. Background-subtracted fluorescence data were expressed as mean±s.e.m. from four technical replicates.

FIGS. 15A-C. Theoretical LoD of the WT LwaCas13a and RBD #3L coupled to the electrochemical sensor in the detection of synthetic RNA target. (FIG. 15A) ΔI % (mean±s.d. from three technical replicates) from FIG. 6A was plotted as a function of target concentration (0, 0.01, 0.1, 0.5, and 1 pM) and fitted by linear regression with replicate y values considered as individual data points. (FIG. 15B) ΔI % (mean±s.d. from three technical replicates) from FIG. 6B was plotted as a function of target concentration (0, 1, 10, 50, and 100 aM) and fitted by linear regression. (FIG. 15C) ΔI % measurement from samples with activated RBD #3L RNP. The sample treatment and assay conditions were in parallel with FIG. 6B except the reaction buffer used was Tris (Table 5). ΔI % (mean±s.d. from three technical replicates) was plotted as a function of target concentration (0, 1, 5, 10, and 20 aM) and fit by linear regression. LoD values were 0.79 pM for WT, 19.76 aM for RBD #3L in HEPES buffer, and 3.28 aM for RBD #3L in Tris buffer, respectively.

FIGS. 16A-D. Optimization of reaction buffer. (Related to FIG. 3). Background-subtracted fluorescence using various reaction buffers after 2-h reaction. The RNPs were formed as described in Methods, the reporter was 125 nM U₁₁. The Mg²⁺ was 5 mM. The target is 10 pM SARS-CoV-2 N gene fragment. Various concentrations and pH of Tris and HEPES buffers were tested. (FIG. 16A) Comparison between two previously used buffering conditions, 20 mM HEPES, pH 6.8, and 40 mM Tris, pH 7.3. (FIG. 16B) Comparison of various pH values, 6.8, 7.0, 7.5 and 8.0. The buffer concentrations were all 20 mM. (FIG. 16C) Comparison of various concentrations of Tris buffer (pH 8.0), 20, 30, 40, 50 and 100 mM. (FIG. 16D) Comparison between 50 mM Tris pH 8.0 buffer and commercial RNaseAlert buffer. For FIGS. 16A-D, means were calculated from three or four technical replicates. The highest values are labeled.

FIGS. 17A-F. BSA, Triton X-100, and salt concentration affect collateral activity. Background-subtracted fluorescence in the detection of SARS-CoV-2 N gene synthetic target ([SEQ ID NO: 33], T1). (FIGS. 17A-E) Bar graphs are the 60-min background-subtracted fluorescence mean±s.e.m. from four technical replicates. The mean values were labeled on the top of each bar. Target concentrations are as indicated. For each group of three bars, the left bar represents RBD #3L, the center bar represents RBD #4L, and the right bar represents WT. (FIG. 17F) Reaction buffer compositions of FIGS. 17A-E.

FIGS. 18A-F. LoD analysis of RBD #3L, RBD #4L and WT LwaCas13a in Tris buffer. (FIGS. 18A-C) Raw fluorescence data measured for 120 min, expressed as mean±s.e.m. from four technical replicates. RBD #3L, RBD #4L, and WT LwaCas13a were complexed with crRNA (cr1) in reaction buffer as in FIG. 17A and tested with 2.5, 1, 0.25, 0.1, 0.025 pM of the synthetic RNA ([SEQ ID NO: 33], T1). The 0 PM target samples were used as no-target control (NTC). (FIG. 18D) Bar plot represented the 120-min background-subtracted fluorescence expressed as mean±s.e.m. from four technical replicates. Adjusted p values were calculated using two-way ANOVA with Dunnett's test: ns, not significant, ***p=0.0008, ****p<0.0001, compared to background signal (NTC). (FIG. 18E) The velocities of each reaction within 60 min were obtained by linear regression from FIGS. 18A-C and were expressed as mean±s.e.m. from four technical replicates and plotted as a function of the target concentration. (FIG. 18F) Analytical LoD of three proteins was calculated using the equation: LoD=3·SD_{NTC}/Slope. SD_{NTC} was the standard derivation of the reaction velocity from the non-target control samples. The slope was determined by linear regression of the reaction velocity against target concentration from FIG. 18E.

FIGS. 19A-B. Sequences related to programmability and specificity tests. (Related to FIG. 4). (FIG. 19A) ssRNA1 target (T3 [SEQ ID NO: 35]) and 11 crRNAs ([SEQ ID NOS: 59-69], cr4-14) tiling along the sequence. (FIG. 19B) Single-point mutations at every two nucleotides (MM2-MM28 [SEQ ID NOS: 37-43 and 126-132], T5-T18) and double-point mutations (DM1-DM25 [SEQ ID NOS: 44-50], T19-T25) at every four nucleotides were introduced to the 28-nt synthetic SARS-CoV-2 N gene fragment target (PM [SEQ ID NO: 36], T4).

FIGS. 20A-E. Kinetic analysis of RBD #3L, RBD #4L, and WT LwaCas13a in reactions supplemented with BSA and Triton X-100. (FIGS. 20A-C) Progress curves of cleaved reporters versus time. Data were plotted as mean±s.e.m. from four technical replicates. The RNP complex of RBD #3L, RBD #4L, and WT in reaction buffer as in FIG. 17B were incubated with 50 pM of the targets ([SEQ ID NO: 33], T1) for 10 min at 37° C. prior to cleavage reaction using 6400, 3200, 1600, 800, 400, 200, 100 and 50 nM of the U₁₁ substrates. The initial velocities (within 300 s) of each reaction were obtained by linear regression. (FIG. 20D) Initial velocities obtained from FIGS. 20A-C were plotted as mean±s.e.m. from four technical replicates versus the Un reporter concentration and fit to a Michaelis-Menten curve. (FIG. 20E) Summary of kinetic parameters obtained from FIG. 20D. Values of k_{cat} and K_{M} were expressed as mean with 95% CI.

FIGS. 21A-D. Electrostatic surface potential and proposed reporter binding patterns. (FIG. 21A) Surface views of the predicted LwaCas13a from FIG. 2B. There are positive charges near the central channel for crRNA and target RNA binding as well as on other regions of the protein surface, which may interact with any surrounding RNAs in a sequence-independent manner. Non-specific RNAs may stabilize the apo-form protein through neutralizing effects and electrostatic interactions (Charles et al., 2021). (FIG. 21B) Reporters in fluorescence Cas13a assay are either at a free state (i) in the solution, or bound with protein. The bound reporters may either bind to active site distal regions unavailable for cleavage (ii), or active site proximal regions ready for cleavage (iii). Inserting an RBD to the active site-proximal Loop 1 would probably increase the proportion of state (iii) RNA reporter and therefore improve the collateral cleavage efficiency. Additional strategies to reduce state (ii) reporters, which may bind to the protein surface without being cleaved due to the distance from active sites, would also increase the collateral cleavage efficiency. (FIGS. 21C-D) Surface views of the AlphaFold (Jumper et al., 2021) predicted RBD #3L (FIG. 21C) and RBD #4L (FIG. 21D). Insets: surface views of RBD #3 (PDB: 5MPL) and RBD #4 (PDB: 2MXY) from FIGS. 2E-F.

FIG. 22. Schematic of CRISPR-Cas13 based electrochemical sensor. The screen-printed electrode (SPE) is comprised of three electrodes, which are gold (Au) as the working electrode (WE) and counter electrode (CE), and Ag as a pseudo-reference electrode (RE). The WE is functionalized by tethering thiol-linked U₂₀ with 3′-methylene blue (MB). The electron transfer between WE and the MB is acquired by square wave voltammetry (SWV). The applied negative potential on the electrode and the negatively charged reporter RNA backbone with close-packed passivator 6-mercapto-1-hexanol (MCH) molecule can lead to the formation of a monolayer of fully extended PolyU lining up on the surface (Nano et al., 2021). This results in a distinct RNA conformation relative to the free RNA reporter in solution, providing a compact interface for protein complex and reporter interaction, which would be restricted to a minimal level and occur only at the protein-reporter interface, effectively reducing the proportion of state (ii) reporter (FIG. 21B). The affinity between fused RBD and the reporters may facilitate protein binding with the immobilized reporters, leading to efficient collateral cleavage of the reporters. In contrast, due to the constrained protein-RNA binding and lack of an RBD, the WT may interact less and in a disorganized manner with the immobilized reporters. Before adding the target, the protein: crRNA complex is not activated, and the tethered reporters will remain intact, resulting in a high electrochemical current of MB. After adding the target, the transduced signal decreases due to the loss of MB by collateral cleavage of the U₂₀ linker. The difference of current (ΔI) before and after adding the target reflects the collateral activity, showing a target concentration-dependent manner of signal change.

FIG. 23. Measurement of surface reporter density. (Related to FIG. 6) The reporter probe density was calculated by using the cyclic voltammetry measurement. Reporter functionalized SPE was scanned between −0.95-0.45 V versus the Hg/Hg₂SO₄ RE at 50 mV/s scan rate. The reduction peak area of methylene blue (4.93×10⁻⁷ AV) was integrated using OriginLab software. After being divided by the scan rate (50 mV/s), the charge (Q) was 9.87×10⁻⁶ C (Q=peark area/scan rate= (4.93×10⁻⁷ AV)/(50 mV/s)=9.87×10⁻⁶ C). The equation calculated the reporter probe amount was Γ=Q/(n·A·F); where n is equal to 2, A is the working electrode surface area (2.0 mm²), and F is the Faraday's constant (96485 C/mol). The reporter density (Γ) was estimated to be 25 pmol/mm². The total amount of functionalized reporter on the WE was estimated to be 50 pmol.

FIGS. 24A-F. Investigation of electrochemical kinetics. (FIG. 24A) Representative images of peak current acquired by SWV scans. The initial peak current (ip₀) was measured with electrolyte buffer containing 10 mM Tris-HCl (pH 8.0) and 100 mM NaCl before adding the CRISPR reaction onto the sensor. The CRISPR reactions were assembled as described in Method and gently dispersed in the electrolyte. Inset is the overlapped responsive peak current measured by consecutive SWV scans with 30 s intervals (ipₜ, t=30, 60, 90 . . . ). (FIG. 24B) Analysis of initial velocity (V₀, nAmp/s) as a function of the initial peak current (ip₀, nAmp). The time course voltammetric responses were recorded every 30 s within 2 min to get a linear relationship between the peak current and the time duration. V₀ was calculated as the change of peak current per second (nAmp/s). The target concentration was 1 pM for both reactions using RBD #3L and the WT. Dots are experimentally measured values. Solid lines are the fitted simple linear regression. Dashed lines represent 95% CI (GraphPad). A linear relationship between V₀ and ip₀ in reactions using RBD #3L implied a first-order kinetics and a constrained reporter-protein interaction. Meanwhile, no obvious curve fitting pattern can be seen between V₀ and ip₀ in reactions using the WT. The difference in V₀-ip₀ relationship between the RBD #3L and the WT revealed that the interactions between the fused RBD and the immobilized reporters could be the major driving force for collateral cleavage of the reporters on the sensor. The severely restricted protein-reporter interactions might lead to unfavorable kinetics and large signal fluctuations in WT-mediated reactions. (FIGS. 24C-E) Time-course curves of electrochemical measurement of 1 pM and 10 aM targets and no-target control (NTC) using the WT (FIG. 24C), RBD #3L (FIG. 24D), and RBD #4L (FIG. 24E). The peak current at each time point was normalized to the corresponding ip₀. The connected circles with error bars in c-e were mean±s.e.m from three or four replicates or mean from two replicates (n=2-4). (FIG. 24F) Fold change of the normalized on-target peak current to the corresponding normalized peak current was mean±s.e.m. of four replicates for WT and RBD #3L, and mean of two replicates for RBD #4L from FIGS. 24C-E. Lines were one-phase decay curve fitting (GraphPad). The SARS-CoV-2 N gene fragment synthetic RNA target was used in FIGS. 24A-F.

FIGS. 25A-C. RT-qPCR quantification of target RNA. (FIG. 25A) Measured Cq values of standard positive controls (IDT) with concentrations of 5000, 1000, 200, 40, 8 and 1.6 copies/μL. (FIG. 25B) Synthetic RNA targets and (FIG. 25C) inactive viruses were serially diluted, measured by RT-qPCR, and interpolated from the standard curve from FIG. 25A. The expected concentration was calculated from stock concentration either determined by using Nanadrop (FIG. 25A), or provided by the vendor's ddPCR analysis (FIG. 25B). (FIGS. 25A-C) Points and errors represented mean±s.d. from three technical replicates.

FIGS. 26A-B. Protein purification of active and inactive RBD #3L, RBD #4L, and WT LwaCas13a. (FIG. 26A) Cation exchange chromatography of LwaCas13a proteins. (FIG. 26B) SDS-PAGE image of purified proteins from a single run.

FIG. 27. Unprocessed SDS-PAGE gel for concentrated RBD #1L-RBD #7L and WT LwaCas13a.

### DETAILED DESCRIPTION

CRISPR-Cas13 has been rapidly developed for nucleic acid-based diagnostics by using its characteristic collateral activity. Despite the recent progress in optimizing the Cas13 system for nucleic acid detection, engineering Cas13 protein with enhanced collateral activity has been challenging, mostly due to its complex structural dynamics. The Leptotrichia wadei (Lwa) Cas13a was engineered by inserting different RNA binding domains (RBDs) to a unique active site-proximal loop within its higher eukaryotes and prokaryotes nucleotide-binding (HEPN) domain. By coupling to reporters with extended length to ensure simultaneous binding and cleavage, two LwaCas13a variants achieved up to 58-fold enhanced collateral activity over the wild-type (WT) in the fluorescence-based assay. When applying the variants on a screen-printed electrochemical device, clear detection of ˜1 attomolar (0.6 copy/μL) of the SARS-CoV-2 genome was achieved without target preamplification, which was 50,000-fold more sensitive than WT. The Cas13a-based amplification-free nucleic acid detection technology provided herein is among the most sensitive diagnostic platforms to date.

Herein, the collateral activity of WT LwaCas13a was improved by structure-guided protein engineering. Collateral activity-enhanced LwaCas13a variants were generated by inserting selected RBDs to the tip of an active site-proximal loop. Two of the most potent variants, RBD #3L and #4L, increased the collateral cleavage rate up to 58-fold with a U₁₁ reporter in fluorescence assays. When further integrated with an electrochemical device, these two variants detected attomolar levels of viral RNA targets without preamplification and improve the detection limit by more than 50,000 folds. This technology utilizes an engineered LwaCas13a enzyme to increase the collateral activity and therefore can be readily integrated to a variety of previously established Cas13a-based detection platforms (Gootenberg et al., 2018; Ackerman et al., 2020; Bruch et al., 2021; Shinoda et al., 2021; Arizti-Sanz et al., 2020).

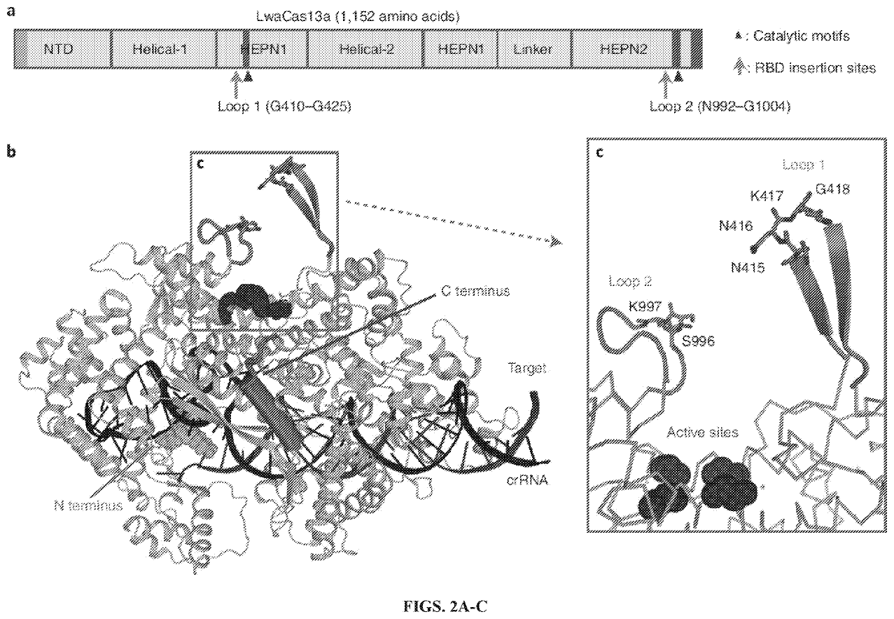

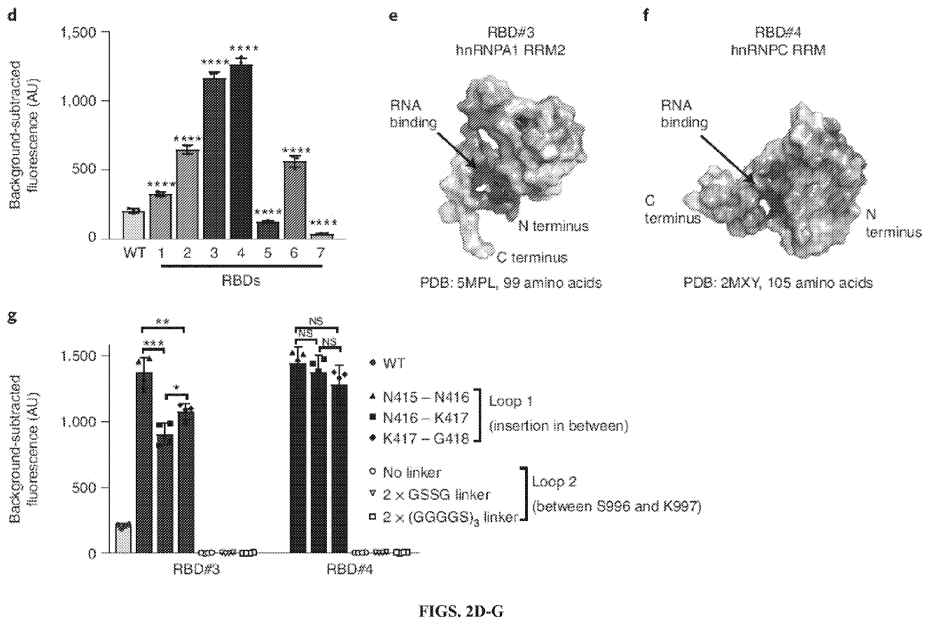

Since the emergence of CRISPR-based nucleic acid detection technology, many attempts have improved reaction sensitivity for fast on-site nucleic acid detection using a variety of approaches, including target preamplification (Gootenberg et al., 2017; Gootenberg et al., 2018), auxiliary proteins with corresponding activator RNA additions (Gootenberg et al., 2018; Beusch et al., 2017), multiple-crRNA combinations (Fozouni et al., 2021; Ooi et al., 2021; Nguyen et al., 2020), reaction optimization (Arizti-Sanz et al., 2020; Nguyen et al., 2020; Joung et al., 2020), field-deployable sample treatment (Myhrvold et al., 2018; Joung et al., 2020; Lee et al., 2020), and versatile sensing devices (Shinoda et al., 2021; Fozouni et al., 2021; Liu et al., 2021; Bruch et al., 2019; Son et al., 2021; Hajian et al., 2019; Dai et al., 2019). However, it has been challenging to engineer the Cas13a protein due to its structural complexity and catalytic dynamics as an active RNase. The previous engineering of Cas enzymes mainly focused on tethering various functional proteins or domains to the N- and/or C-terminus of a Cas effector (Cox et al., 2017; Abudayyeh et al., 2019). The LwaCas13a terminus can be buried (FIG. 2B), and linking additional functional domains/motifs to these termini leads to a detrimental effect on Cas13a collateral activity.

However, fusing selected RBDs into a B-hairpin loop proximal to the enzyme's active site enhanced RNA binding. cleavage, and enzyme stability. A few studies have employed a domain-insertional strategy to add new functionalities to Cas9, such as inserting a ligand-binding domain for the allosteric regulation (Oakes et al., 2016) or a deaminase to broaden the activity window of base editors (Chu et al., 2021); however, these studies did not show enhancement of activity or substrate binding affinity of Cas9 (Oakes et al., 2016; Chu et al., 2021). The present strategy exploited the RNA binding affinity of selected RRMs and structure-guided domain insertion to dramatically enhance collateral activity while maintaining the programmability and specificity for the target RNA.

There are a large number of known RBDs, including RRMs, DRBMs, and zinc-finger RNA domains, which interact with RNA in a non-sequence-specific manner, especially under physiological conditions where interacting RNAs are present at low abundance (Hentze et al., 2018; Jolma et al., 2020; Dominguez et al., 2018). The studies presented herein provide for the fusion of other regulatory/functional domains to other structural loops proximal to the active sites of Cas13 enzymes. Further exploration of the

RBD repertoire may lead to the discovery of more active and robust Cas13a variants. In addition to diagnostic context, these engineered Cas13a proteins could potentially be used for the detection of agricultural pathogens and sensing of proteins and small molecules when in combination with other biotechnologies. The present case study on LwaCas13a points to a new direction for future engineering studies on other Cas enzymes, which are usually large and may contain other potential structural loops proximal to their active sites for fusing other regulatory/functional domains for both in vitro and in vivo applications.

Notably, the inclusion of the engineered RBD-LwaCas13a fusion proteins into a Uzo RNA redox reporter-functionalized SPE device enabled the detection of heat-inactivated SARS-CoV-2 viral genome at attomolar sensitivity and successfully distinguished the clinical SARS-CoV-2 positive samples from negatives, without requiring target preamplification. The engineered CRISPR-Cas13a system is among the most sensitive CRISPR-based amplification-free nucleic acid detection technologies developed to date (LoD 0.6 copy/μL, within 30 min). It shows comparable sensitivity to the droplet-based Cas13a kinetic barcoding (below 1 copy/μL), which contained a total of 26 crRNAs in one single assay (Son et al., 2021), it is one order of magnitude more sensitive than that of the Cas13a-Csm6 tandem assay (31 copies/μL), which required eight different crRNAs and a Csm6 protein with its chemically modified activator RNA (Liu et al., 2021); two orders of magnitude more sensitive than the Cas13a-mobile phone microscopy (200 copies/μL), which utilized three different crRNAs (Fozouni et al., 2021); three orders of magnitude more sensitive than the Cas13-microchamber array (Shinoda et al., 2021) and immobilized Cas9-graphene field-effect transistor CRISPR-chip (Hajian et al., 2019), and six orders of magnitude more sensitive than the CRISPR-electrochemical biosensor that depends on WT Cas13a protein (Bruch et al., 2019). This LwaCas13a engineering technology holds great potential for ultrasensitive detection of various RNAs of clinical and environmental importance.

### I. CRISPR-Cas13 Systems and Methods of Use

Microbial Clustered Regularly Interspaced Short Palindromic Repeats (CRISPR) and CRISPR-associated (CRISPR-Cas) adaptive immune systems contain programmable endonucleases, such as Cas9 and Cpf1, which both target DNA. Single effector RNA-guided RNases, including Cas13a, provide a platform for specific RNA sensing. These RNA-guided RNases can be easily and conveniently reprogrammed using CRISPR RNA (crRNAs) to cleave target RNAs. Unlike the DNA endonucleases Cas9 and Cpf1, which cleave only its DNA target, RNA-guided RNases, like Cas13a, remains active after cleaving its RNA target, leading to “collateral” cleavage of non-targeted RNAs in proximity. This crRNA-programmed collateral RNA cleavage activity provides for the use of RNA-guided

RNases to detect the presence of a specific RNA by triggering degradation of a nonspecific reporter RNA, which can serve as a readout.

The composition and methods disclosed herein use engineered RNA targeting effectors to provide a robust CRISPR-based diagnostic with attomolar sensitivity. The compositions disclosed herein can be prepared in freeze-dried or lyophilized formats for convenient distribution and point-of-care (POC) applications. Such embodiments are useful in multiple scenarios in human health including, for example, viral detection, bacterial strain typing, sensitive genotyping, and detection of disease-associated cell free DNA or RNA.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target RNA may be a RNA polynucleotide or a part of a RNA polynucleotide to which a part of the crRNA, i.e., the guide sequence, is designed to have complementarity and to which the effector function mediated by the complex comprising CRISPR effector protein and a gRNA is to be directed.

The Cas13 enzyme used herein may be a Cas13a nuclease, a Cas13b nuclease, or a Cas13d nuclease. The Cas13 enzyme used herein may have a sequence homology or identity of at least 30%, or at least 40%, or at least 50%, or at least 60%, or at least 70%, or at least 80%, more preferably at least 85%, even more preferably at least 90%, such as for instance at least 95% with the sequence of SEQ ID NO: 1. The Cas13 enzyme may be any of Leptotrichia wadei (Lwa) Cas13a, Leptotrichia wadei (F0279) Cas13a, Leptotrichia buccalis (Lbu) Cas13a, Leptotrichia shahii Cas13a, Lachnospiraceae bacterium MA2020 Cas13a, Lachnospiraceae bacterium NK4A179 Cas13a, Clostridium aminophilum (DSM 10710) Cas13, Carnobacterium gallinarum (DSM 4847) Cas13a, Paludibacter propionicigenes (WB4) Cas13a, Listeria weihenstephanensis (FSL R9-0317) Cas13a, Listeriaceae bacterium (FSL M6-0635) Cas13a, Listeria newyorkensis (FSL M6-0635) Cas13a, Rhodobacter capsulatus (SB 1003) Cas13a, Rhodobacter capsulatus (R121) Cas13a, Rhodobacter capsulatus (DE442) Cas13a, Listeria seeligeri Cas 13a, Sinomicrobium oceani (WP_072310476.1) Cas13b, Prevotella intermedia Cas13b, A2033_10205 [Bacteroidetes bacterium GWA2_31_9] (OFX18020.1) Cas13b, SAMN05421542_0666 [Chryseobacterium jejuense] (SDI27289.1) Cas13b, SAMN05444360_11366 [Chryseobacterium carnipullorum] (SHM52812.1) Cas13b, SAMN05421786_1011119 [Chryseabacterium ureilyticum] (SIS70481.1) Cas13b, Reichenbachiella agariperforans (WP_073124441.1) Cas13b, Porphyromonas gulae Cas13b (accession number WP_039434803), Prevotella sp. P5-125Cas13b (accession number WP_044065294), Porphyromonas gingivalis Cas13b (accession number WP_053444417), Porphyromonas sp. COT-052 OH4946 Cas13b (accession number WP_039428968), Bacteroides pyogenes Cas13b (accession number WP_034542281), Riemerella anatipestifer Cas13b (accession number WP_004919755), Eubacterium siraeum Cas13d, Ruminococcus sp. Cas13d, Ruminococcus flavefaciens FD1 Cas13d, Ruminococcus albus Cas13d, Ruminococcus bicirculans Cas13d, or Ruminococcus_sp_CAG57 Cas13d.

The activity of Cas13a may depend on the presence of two higher eukaryotes and prokaryotes nucleotide-binding (HEPN) domains. These have been shown to be RNase domains, i.e., nuclease domains (in particular an endonuclease), that cut RNA. Cas13a HEPN may also target DNA, or potentially DNA and/or RNA. On the basis that the HEPN domains of Cas13a are at least capable of binding to and, in their wild-type form, cutting RNA, then it is preferred that the Cas13a effector protein has RNase function. The Cas protein may be a Cas13a ortholog of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. The species of organism of such a genus can be as otherwise herein discussed.

The Cas13 protein as referred to herein may also encompass a functional variant of Cas13 or a homologue or an orthologue thereof. A “functional variant” of a protein as used herein refers to a variant of such protein which retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made.

Functional variants of Cas13 may include a linker positioned between an insertion or fusion and the Cas13 enzyme. Flexible linkers generally are comprised of helix- and turn-promoting amino acid residues such as alanine, serine and glycine. However, other residues can function as well. For example, the linker may have proline, arginine, glutamic acid, and lysine residues. The linker can comprise an amino acid residue, e.g., 1, 2, 3, 4, 5, 6, 7, 8, 9, or 10 amino acid residues. Typically, the linker will comprise less than 10, 20, or 30 amino acid residues. Suitable linkers include: [Gly-Ser]ₓ, wherein x is 1, 2, 3, 4, 5, 6, 7, 8, 9, or 10; [Gly-Gly-Ser]ₓ, wherein x is 1, 2, 3, 4, 5, 6, 7, 8, 9, or 10; [Gly-Gly-Ser]; (GSAGSAAGSGEF)ₓ, wherein x is 1, 2, 3 or 4; (SIVAQLSRPDPA)ₓ, wherein x is 1, 2, 3 or 4; or an XTEN sequence, or a sequence that differs therefrom by no more than 1, 2, 3, 4, 5, 6, 7, 8, 9 or 10 amino acid residues.

The sequences of proteins may be modified for a variety of reasons, such as improved expression, improved cross-reactivity, or diminished off-target binding. Modified proteins may be made by any technique known to those of skill in the art, including expression through standard molecular biological techniques. For example, one may wish to make modifications, such as introducing conservative changes into a protein. In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

As used herein, the term “crRNA” or “guide RNA” or “single guide RNA” or “gRNA” refers to a polynucleotide comprising any polynucleotide sequence having sufficient complementarity with a target nucleic acid sequence to hybridize with the target nucleic acid sequence and to direct sequence-specific binding of a RNA-targeting complex comprising the crRNA and a CRISPR effector protein to the target nucleic acid sequence. In general, a crRNA may be any polynucleotide sequence (i) being able to form a complex with a CRISPR effector protein and (ii) comprising a sequence having sufficient complementarity with a target polynucleotide sequence to hybridize with the target sequence and direct sequence-specific binding of a CRISPR complex to the target sequence.

As used herein the term “capable of forming a complex with the CRISPR effector protein” refers to the crRNA having a structure that allows specific binding by the CRISPR effector protein to the crRNA such that a complex is formed that is capable of binding to a target nucleic acid in a sequence specific manner and that can exert a function on said target nucleic acid. Structural components of the crRNA may include direct repeats and a guide sequence (or spacer). The sequence specific binding to the target nucleic acid is mediated by a part of the crRNA, the “guide sequence”, being complementary to the target nucleic acid. As used herein the term “wherein the guide sequence is capable of hybridizing” refers to a subsection of the crRNA having sufficient complementarity to the target sequence to hybridize thereto and to mediate binding of a CRISPR complex to the target nucleic acid. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net).

The CRISPR system as provided herein may make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

crRNAs may be chemically modified. Examples of crRNA chemical modifications include, without limitation, incorporation of 2′-O-methyl (M), 2′-O-methyl 3′-phosphorothioate (MS), or 2′-O-methyl 3′-thioPACE (MSP) at one or more terminal nucleotides. Such chemically modified crRNAs can comprise increased stability and increased activity as compared to unmodified crRNAs, though on-target vs. off-target specificity is not predictable. Chemically modified guide crRNAs further include, without limitation, RNAs with phosphorothioate linkages, locked nucleic acid (LNA) nucleotides comprising a methylene bridge between the 2′ and 4′ carbons of the ribose ring, 2′-fluoro RNA and crRNA with DNA substitution.

A crRNA may be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. A guide sequence may be less than about 75, 50, 45, 40, 35, 30, 25, 20, 15, 12, or fewer nucleotides in length. Preferably the guide sequence is 10 to 30 nucleotides long. The ability of a guide sequence to direct sequence-specific binding of a CRISPR complex to a target sequence may be assessed by any suitable assay. For example, cleavage of a target RNA may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence, and hence a nucleic acid-targeting guide RNA may be selected to target any target nucleic acid sequence. In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the crRNA, i.e. the guide sequence, is designed to have complementarity and to which the effector function mediated by the complex comprising CRISPR effector protein and a crRNA is to be directed.

A target sequence may be any DNA sequence. A target sequence may be any RNA sequence. The target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nuclear RNA (snoRNA), double stranded RNA (dsRNA), non-coding RNA (ncRNA), long non-coding RNA (IncRNA), small cytoplasmic RNA (scRNA), or viral genomic RNA. The target sequence may be a sequence within an RNA molecule selected from the group consisting of mRNA, pre-mRNA, rRNA, and tRNA. The target sequence may be a sequence within an RNA molecule selected from the group consisting of ncRNA and IncRNA. The target sequence may be a sequence within an mRNA molecule or a pre-mRNA molecule.

A nucleic acid-targeting guide RNA may be selected to reduce the degree of

secondary structure within the RNA-targeting guide RNA. For example, about or less than about 75%, 50%, 40%, 30%, 25%, 20%, 15%, 10%, 5%, 1%, or fewer of the nucleotides of the nucleic acid-targeting guide RNA may participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker & Stiegler (Nucleic Acids Res. 9:133-48, 1981). Another example folding algorithm is the online webserver RNAfold, developed at Institute for Theoretical Chemistry at the University of Vienna, using the centroid structure prediction algorithm (see, e.g., Gruber et al., Cell 106:23-24, 2008; and Carr & Church, Nature Biotechnology 27:1151-62, 2009).

A crRNA may comprise, consist essentially of, or consist of a direct repeat (DR) sequence and a guide sequence or spacer sequence. In certain embodiments, the crRNA may comprise, consist essentially of, or consist of a direct repeat sequence fused or linked to a guide sequence or spacer sequence. The direct repeat sequence may be located upstream (i.e., 5′) from the guide sequence or spacer sequence. The direct repeat sequence may be located downstream (i.e., 3′) from the guide sequence or spacer sequence. The crRNA may comprise a stem loop, such as a single stem loop. The direct repeat sequence may form a stem loop, such as a single stem loop.

The term “RNA reporter,” as used herein, refers to a molecule that can be cleaved by an activated CRISPR system effector protein described herein. An un-cleaved RNA reporter prevents the generation or detection of a positive detectable signal. A positive detectable signal may be any signal that can be detected using optical, fluorescent, chemiluminescent, electrochemical, or other detection methods known in the art. The RNA reporter may prevent the generation of a detectable positive signal or mask the presence of a detectable positive signal until the masking construct is cleaved. The term “positive detectable signal” is used to differentiate from other detectable signals that may be detectable in the presence of the RNA reporter. For example, a first signal may be detected when the masking agent is present (i.e. a negative detectable signal), which then converts to a second signal (e.g. the positive detectable signal) upon detection of the target molecules and cleavage of the RNA reporter by the activated CRISPR effector protein.

The RNA reporter may comprise an RNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA reporter may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of the methods provided herein, only that selection of the fluorophore/quencher pairs ensures masking of the fluorophore. Upon activation of the effector proteins disclosed herein, the RNA reporter is cleaved thereby severing the proximity between the fluorophore and quencher needed to maintain the contact quenching effect. Accordingly, detection of the fluorophore may be used to determine the presence of a target molecule in a sample.

The RNA reporter may comprise an RNA oligonucleotide to which is attached a redox active molecule. The RNA reporter may be covalently bound to a working electrode. Release of the redox active molecule upon cleavage of the RNA reporter will release the redox active molecule into the solution and away from the working electrode. A change (i.e., a decrease) in the signal of the redox active material as a result of the cleavage of the RNA reporter can be measured. Such redox active molecules can be any molecule capable of electron transfer under certain conditions. For example, redox active molecules include transition metal complexes and organic electron transfer moieties. Transition metals include those whose atoms have a partial or complete d shell of electrons; elements having the atomic numbers 21-30, 39-48, 57-80 and the lanthanide series. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinium (Pt), scandium (Sc), titanium (Ti), vanadium (V), chromium (Cr), manganese (Mn), nickel (Ni), molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinium, cobalt and iron. These organic molecules include, but are not limited to, riboflavin, xanthene dyes, azine dyes, acridine orange, N,N′-dimethyl-2,7-diazapyrenium dichloride (DAP2+), methylviologen, ethidium bromide, quinones such as N,N′-dimethylanthra(2 1,9-def: 6,5, 10-d′e′f′)diisoquinoline dichloride (ADIQ2+); porphyrins ([meso-tetrakis(N-methyl-x-pyridinium)porphyrin tetrachloride], varlamine blue B hydrochloride, Bindschedler's green; 2,6-dichloroindophenol, 2,6-dibromophenolindophenol; Brilliant crest blue (3-amino-9-dimethyl-amino-10-methylphenoxyazine chloride), methylene blue; Nile blue A (aminoaphthodiethylaminophenoxazine sulfate), indigo-5,5′,7,7′-tetrasulfonic acid, indigo-5,5′,7-trisulfonic acid; phenosafranine, indigo-5-monosulfonic acid; safranine T; bis(dimethylglyoximato)-iron(II) chloride; induline scarlet, neutral red, anthracene, coronene, pyrene, 9-phenylanthracene, rubrene, binaphthyl, DPA, phenothiazene, fluoranthene, phenanthrene, chrysene, 1,8-diphenyl-1,3,5,7-octatetracene, naphthalene, acenaphthalene, perylene, TMPD and analogs and substituted derivatives of these compounds.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. An LED and other electronic measuring or sensing devices may be included in the LOC-RFID chip.

The RNA reporter may be a conductive RNA molecule. The conductive RNA molecule may be attached to the conductive material. Conductive molecules can be conductive nanoparticles, conductive proteins, metal particles that are attached to the protein or latex or other beads that are conductive. The release of the conductive molecules may be detected across a sensor. The assay may be a one step process.

As demonstrated herein, the CRISPR effector systems are capable of detecting down to attomolar concentrations of target molecules. Due to the sensitivity of said systems, a number of applications that require the rapid and sensitive detection may benefit from the embodiments disclosed herein.

The compositions and methods disclosed herein may be used to detect the presence of one or more microbial agents in a sample, such as a biological sample obtained from a subject. In certain example embodiments, the microbe may be a bacterium, a fungus, a yeast, a protozoa, a parasite, or a virus. Accordingly, the compositions and methods disclosed herein can be adapted for use in other methods, or in combination with other methods, that require quick identification of microbe species, monitoring the presence of microbes, detection of certain phenotypes (e.g. bacterial resistance), and/or monitoring of disease progression and/or outbreak. Because of the rapid and sensitive diagnostic capabilities of the compositions and methods disclosed herein, detection of microbe species type, down to a single nucleotide difference, and the ability to be deployed as a POC device, the compositions and methods disclosed herein may be used guide therapeutic regimens, such as selection of the appropriate antibiotic or antiviral. The embodiments disclosed herein may also be used to screen environmental samples (air, water, surfaces, food etc.) for the presence of microbial contamination.

The compositions and methods disclosed herein may identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods and systems of the invention, by employing the use of more than one effector protein, wherein each effector protein targets a specific microbial target sequence. In this way, a multilevel analysis can be performed for a particular subject in which any number of microbes can be detected at once. Simultaneous detection of multiple microbes may be performed using a set of probes that can identify one or more microbial species.

A method for detecting microbes in samples is provided, comprising distributing a sample or set of samples into one or more individual discrete volumes, the individual discrete volumes comprising a CRISPR system as described herein; incubating the sample or set of samples under conditions sufficient to allow binding of the one or more guide RNAs to one or more microbe-specific targets; activating the CRISPR effector protein via binding of the one or more crRNAs to the one or more target molecules, wherein activating the CRISPR effector protein results in cleavage of the RNA reporter such that a detectable positive signal is generated; and detecting the detectable positive signal, wherein detection of the detectable positive signal indicates a presence of one or more target molecules in the sample. The one or more target molecules may be RNA comprising a target nucleotide sequence that may be used to distinguish two or more microbial species/strains from one another. The crRNAs may be designed to detect target sequences. The methods disclosed herein may also use certain steps to improve hybridization between crRNA and target RNA sequences.

The methods disclosed herein may also be used to screen environmental samples for contaminants by detecting the presence of a target nucleic acid. As described herein, a sample for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. Environmental samples or biological samples may be crude samples and/or the one or more target molecules may not be purified or amplified from the sample prior to application of the method.

Appropriate samples for use in the methods disclosed herein include any conventional biological sample obtained from an organism or a part thereof, such as a plant, animal, bacteria, and the like. The biological sample may be obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacteria or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g, using formalin) and/or embedding in wax (such as formalin-fixed paraffin-embedded (FFPE) tissue samples). It will appreciated that any method of obtaining tissue from a subject can be utilized, and that the selection of the method used will depend upon various factors such as the type of tissue, age of the subject, or procedures available to the practitioner. Standard techniques for acquisition of such samples are available in the art.

The following provides an example list of the types of microbes that might be detected using the embodiments disclosed herein. The microbe may be a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobrid), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcussp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticusand Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellular e, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi {formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enter ocolitica, Yersinia pestis, and Yersinia pseudotuberculosis) and Xanthomonas maltophilia among others.

The microbe may be a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys(such as Stachybotrys chartarum), Mucroymcosis, Sporothrix, fungal eye infections ringworm, Exserohilum, Cladosporium. The fungus may be a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

The microbe may be a protozoan. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malar iae, and Toxoplasma gondii. Babesia microti, Cryptosporidium parvum, Cyclospora cayetanensis, Plasmodium falciparum, P. vivax, P. ovale, P. malariae, and Toxoplasma gondii.

The microbe may be a parasite. Examples of parasites that can be detected in accordance with disclosed methods include without limitation one or more of (or any combination of), an Onchocerca species and a Plasmodium species.

The compositions and methods disclosed herein may be directed to detecting viruses in a sample. The embodiments disclosed herein may be used to detect viral infection (e.g. of a subject or plant), or determination of a viral strain, including viral strains that differ by a single nucleotide polymorphism. The virus may be a DNA virus, a RNA virus, or a retrovirus. Non-limiting example of viruses useful with the present methods include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. The viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gential-associated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanses encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2Y225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Procine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

The virus may be a retrovirus. Example retroviruses that may be detected using the methods disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

The virus may be a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, and Rhizidovirus.

The compositions and methods disclosed herein may be used for biomarker detection. For example, the compositions and methods disclosed herein may be used for SNP detection and/or genotyping. The compositions and methods disclosed herein may be also used for the detection of any disease state or disorder characterized by aberrant gene expression. Aberrant gene expression includes aberration in the gene expressed, location of expression and level of expression. Multiple transcripts or protein markers related to cardiovascular, immune disorders, and cancer among other diseases may be detected. The methods disclosed herein may be used for cell free DNA detection of diseases that involve lysis, such as liver fibrosis and restrictive/obstructive lung disease. The methods may be used for faster and more portable detection for pre-natal testing of cell-free DNA. The methods disclosed herein may be used for screening panels of different SNPs associated with, among others, cardiovascular health, lipid/metabolic signatures, ethnicity identification, paternity matching, human ID (e.g. matching suspect to a criminal database of SNP signatures). The methods disclosed herein may also be used for cell free DNA detection of mutations related to and released from cancer tumors. The methods disclosed herein may also be used for detection of meat quality. for example, by providing rapid detection of different animal sources in a given meat product. The methods disclosed herein may also be used for the detection of GMOs or gene editing related to DNA. As described herein elsewhere, closely related genotypes/alleles or biomarkers (e.g. having only a single nucleotide difference in a given target sequence) may be distinguished by introduction of a synthetic mismatch in the crRNA.

### II. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials & Methods

Plasmid construct design and molecular cloning. Sequences encoding seven candidate RBDs (#1-#7), including ADAR1⁷⁰⁸⁻⁸⁰¹, hnRNPA1¹⁻¹⁰⁴, hnRNPA⁹⁸⁻¹⁹⁶, hnRNPC²⁻¹⁰⁶, METTL3²⁵⁹⁻³⁵⁷, ADAR1¹²⁶⁻²⁰², and ADAR1²⁹³⁻³⁶⁶ were amplified from HEK293T complementary DNA (cDNA). First, total RNA was extracted by Trizol (Invitrogen, 15596026) reagent per the manufacturer's protocols, treated with RNase-free DNase I (New England Biolabs, M0303S) at 37° C. for 30 min to remove genomic DNA, and heated at 70° C. for 5 min to inactivate DNase activity. To generate cDNA from the RNA extracts, RevertAid First Strand cDNA Synthesis Kit (Thermo Scientific, K1622) was used per the manufacturer's protocols. The RBD-encoding sequences were amplified with Q5 High-Fidelity DNA polymerase (New England Biolabs, M0491L) in a 20-μL PCR reaction, comprising 2 μL of template cDNA, 0.5 μM of forward and reverse primers (Integrated DNA Technologies) with designed 5′ flanking bases and a Bsal restriction site. PCR products were electrophoresed on 1.5% agarose gel with 0.01% (v:v) ethidium bromide in 1×TAE buffer, visualized under a blue-light transilluminator (Accuris) to excise the bands, and purified by QIAquick Gel Extraction Kit (Qiagen, 28076).

Purified DNA pieces, RBD alone or RBD with linkers, were Golden Gate-cloned to an expression vector of LwaCas13a (pC013-Twinstrep-SUMO-huLwCas13a, Addgene plasmid #90097), a gift from Feng Zhang. A silent mutation was introduced to the vector to eliminate the BsaI restriction site for cloning. N-terminal His-tagged SUMO protease expression plasmid was constructed by amplifying the gene sequence encoding yeast Ulp1⁴⁰³⁻⁶²¹ from 1-μL of Saccharomyces cerevisiae strain BY4741 competent cells and Golden Gate-cloning into the expression vector of LwaCas13a to replace the twin-strep tag. SUMO tag, and LwaCas13a sequences. The assembled plasmids were transformed to One-Shot Stb13 Chemically Competent E. coli cells (Thermo Scientific, C737303) and grown overnight on a Luria Broth (LB) agar plate containing 100 μg/mL ampicillin. Colonies containing the correct plasmids, as confirmed by sanger sequencing (Genewiz), were grown in ampicillin-supplement LB medium. The plasmids were purified with QIAprep Spin Miniprep Kit (Qiagen, 27106) and the concentration and purity were measured by a NanoDrop One microvolume Spectrophotometer (Thermo Scientific).

Protein purification. Expression and purification of WT LwaCas13a and RBD fusion proteins were performed as previously described with modifications (Kellner et al., 2019). Briefly, the protein expression vector was transformed into E. coli BL21(DE3) competent cells and grown on LB agar plate overnight. A single colony was picked and inoculated in a 5 mL starter culture, grown overnight, and transferred to a flask containing 1 L LB medium. The cultures were grown at 37° C. and 220 rpm until OD₆₀₀ reached 0.4. The cells were cooled on ice for 30 min, induced with 250 μM IPTG, and grown for 16 h at 16° C. and 160 rpm. Cell pellets were collected by centrifugation at 5,200 g for 20 min and stored at −80° C. before purification.

All protein purification steps were performed at 4° C. or on ice. The cell pellet was resuspended in lysis buffer (50 mM Tris-HCl pH 8.0, 1 M NaCl, 10 mM Imidazole, 1 mM Tris (2-carboxyethyl) phosphine (TCEP)) supplemented with EDTA-free protease inhibitor tablets (Thermo Scientific, A32965) and mixed by rotation until homogeneous. The cells were then disrupted by sonication (Branson) and the cell lysate was centrifuged at 13,000 g for 30 min. The supernatant was incubated with Ni-NTA Agarose resin (UBPBio, P3020) and rotated for 30 min. The protein-bound resin was applied to a gravity-flow column for washing and elution. Most of the non-specifically bound proteins were removed by washing buffer 1 (50 mM Tris-HCl, 1 M NaCl, 20 mM Imidazole, 1 mM TCEP, pH 8.0) and 0.5-1 volume of washing buffer 2 (50 mM Tris-HCl, 1 M NaCl, 20 mM Imidazole, 1 mM TCEP, pH 8.0). The protein was eluted using elution buffer (50 mM Tris-HCl, 0.6 M NaCl, 150 mM Imidazole, 1 mM TCEP, pH 8.0). Notably, the TCEP and protease inhibitor were added freshly before use. The SUMO-tag was removed by mixing the elute with lab-purified SUMO protease and dialyzed overnight against dialysis buffer (50 mM Tris-HCl, 0.6 M NaCl, 2 mM DTT, pH 7.5). The cleaved products were applied to Ni-NTA resins to remove the uncleaved protein and the his-tagged SUMO protease. The collected elute was buffer exchanged to storage buffer (50 mM Tris-HC1, 600 mM NaCl, 5% Glycerol, and 2 mM DTT, pH 7.5) and concentrated to ˜1.5 mg/mL using an Amicon Ultra centrifugal filter unit with 100 kDa cutoff (Millipore, UFC910024). The protein was aliquoted and stored at −80° C. before use. The His-tagged SUMO protease was purified similarly, except washing buffers 1 and 2 contained 10 mM and 20 mM imidazole, respectively.

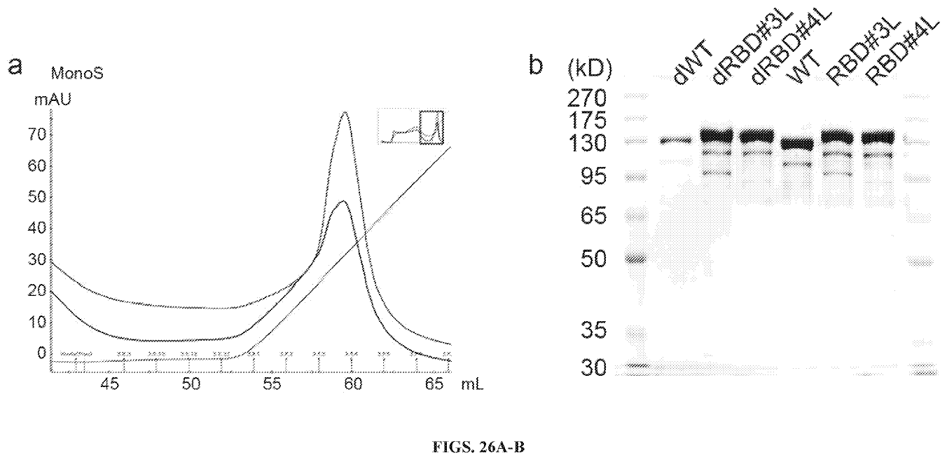

Cation exchange chromatography was performed for further purification of RBD #3L, RBD #4L, WT, and their HEPN2 domain mutants (R1046A/H1051A). After overnight dialysis, proteins were loaded onto a MonoS cation exchange column (Cytiva) via fast protein liquid chromatography (AKTA PURE, GE Healthcare) and eluted over a salt gradient from 300 mM to 1 M NaCl in buffer containing 20 mM Tris-HCl (pH 8.0) and 2 mM DTT. Fractions were collected and visualized by SDS-PAGE with Coomassie blue staining for the presence of desired proteins. Pooled fractions were buffer exchanged and concentrated to ˜15 μM. Proteins were stored in buffer containing 20 mM Tris-HCl pH 7.5, 600 mM NaCl, 2 mM DTT, and 35% glycerol, aliquoted, flash-frozen in liquid nitrogen, and stored at −80° C. before use (FIGS. 26A-B).

RNA target and crRNA preparation. Non-labeled RNA targets ([SEQ ID NOS: 33-53, 126-133], T1-T29, Table 1) and crRNAs ([SEQ ID NOS: 59-74], cr4-cr19, Table 2) were obtained by in vitro transcription (IVT) using the HiScribe T7 Quick High Yield RNA Synthesis kit (NEB, E2050S). IVT templates for T1-T3 and T27-T29 ([SEQ ID NOS: 33-35 and 51-53]) were PCR amplified from gBlock (Integrated DNA Technologies) containing a T7 promoter sequence, and templates for crRNAs were PCR amplified using a universal forward primer ([SEQ ID NO: 79], Pr01) and a reverse primer ([SEQ ID NO: 80-93], Pr02-Pr17, Table 4). PCR products were gel purified (Qiagen) and eluted by nuclease-free water. The concentration and purity of the templates were measured by Nanodrop. At least 1 pmol of DNA was added to each IVT reaction.

IVT templates for T4-T26 ([SEQ ID NOS: 36-50, 126-133]) were obtained by annealing the top primer Pr18 ([SEQ ID NO: 96]) with the bottom primer Pr38 ([SEQ ID NO: 136]), or annealing top primer Pr22 ([SEQ ID NO: 100]) with bottom primer Pr23-Pr37 ([SEQ ID NOS: 101-113, 134-135]) or Pr39-Pr44 ([SEQ ID NOS: 114-115, 137-140]) (Table 4). Briefly, a final concentration of 10 μM of top and bottom primers were added to a 10-μL reaction containing 1 μL 10×Standard Taq buffer (NEB, B9014S). Annealing was performed in a thermocycler by heating the oligos to 95° C. and cooling down to room temperature at 1° C./min. The 10 μL annealing reactions were directly used as IVT templates.

A 40-μl IVT reaction was performed by mixing DNA template with 2.5 mM nucleoside triphosphates, 2 μl T7 polymerase, and 1 U μl Murine RNase Inhibitor (NEB, M0314L), followed by incubation at 37° C. for 4 h. The IVT products were treated with DNase I and purified with either RNAClean XP beads (Beckman Coulter, A63987) (for T4-T25, [SEQ ID NOS: 36-50, 126-132]), or by urea-PAGE gel electrophoresis followed by acid phenol-chloroform extraction. In brief, bands were excised, crushed, and suspended in five volumes of 0.3 M NaOAc (pH 5.2, Thermo Scientific, R1181), and subjected to four repeated cycles of 15-min freezing at −80° C. and quick thawing at room temperature. The elute was filtered and mixed with an equal volume of phenol:chloroform:iso-amyl alcohol (125:24:1, pH 4.5; Sigma-Aldrich, P1944), then centrifuged at 13,000 g and 4° C. for 10 min. The upper aqueous phase was re-extracted by an equal volume of chloroform:iso-amyl alcohol (24:1; Sigma-Aldrich, C0549) twice. For every 400 μl of washed upper phase, 1 μl RNA-grade glycogen (Thermo Scientific, R0551) was added as an inert carrier of RNAs, then mixed thoroughly with 400 μl of isopropanol to precipitate at −20° C. for 1 h. The resulting RNA pellet was washed with 70% ice-cold ethanol twice, air-dried, and redissolved in nuclease-free H₂O. The RNA concentration and purity were measured with Nanodrop and the identity was confirmed by denaturing gel electrophoresis.

The body-radiolabeled target RNAs were in vitro transcribed in each 20-μl reaction containing 50 ng/μl DNA template, 2 mM each nucleoside triphosphate, and 5 U/μl T7 polymerases in reaction buffer (100 mM HEPES-KOH, pH 7.5, 30 mM DTT, and 2 mM spermidine supplemented with 0.34 μM α-[³²P]-ATP (3,000 Ci mmol−1; PerkinElmer, BLU003H250UC)), with 20 mM or 8 mM MgCl₂ for target RNA T1 ([SEQ ID NO: 33]) or T26 ([SEQ ID NO: 133]), respectively. Reactions were incubated at 37° C. for 3 h and subsequently purified with Illustra MicroSpin G-50 columns (GE Healthcare, 27-5330-02), and eluted with 20 μl TE buffer (20 mM Tris-HCl, pH 8.0, and 1 mM EDTA). The concentration of body-radiolabeled target RNAs was measured with Nanodrop. The eluted RNA was aliquoted and stored at −20° C. if not immediately used.

Targets, crRNAs, and IVT primers are listed in Tables 1, 2, and 4, respectively.

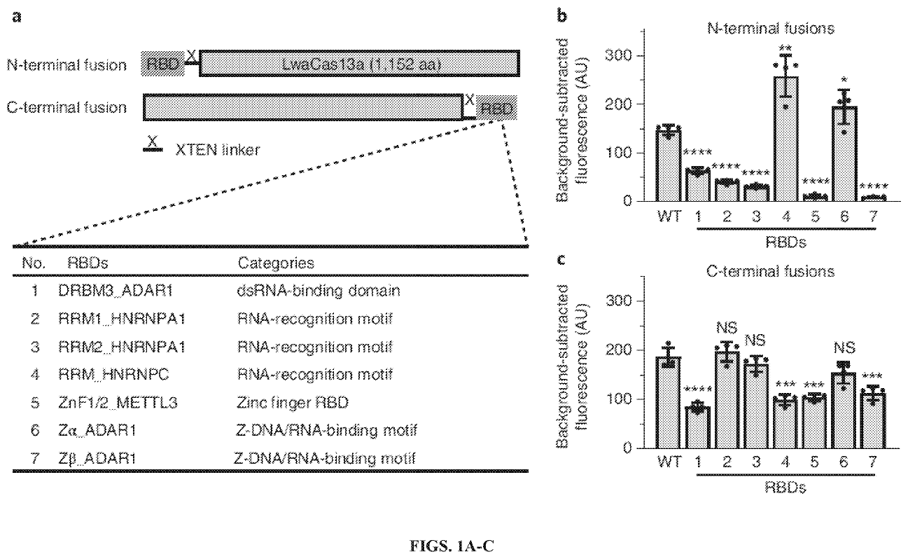

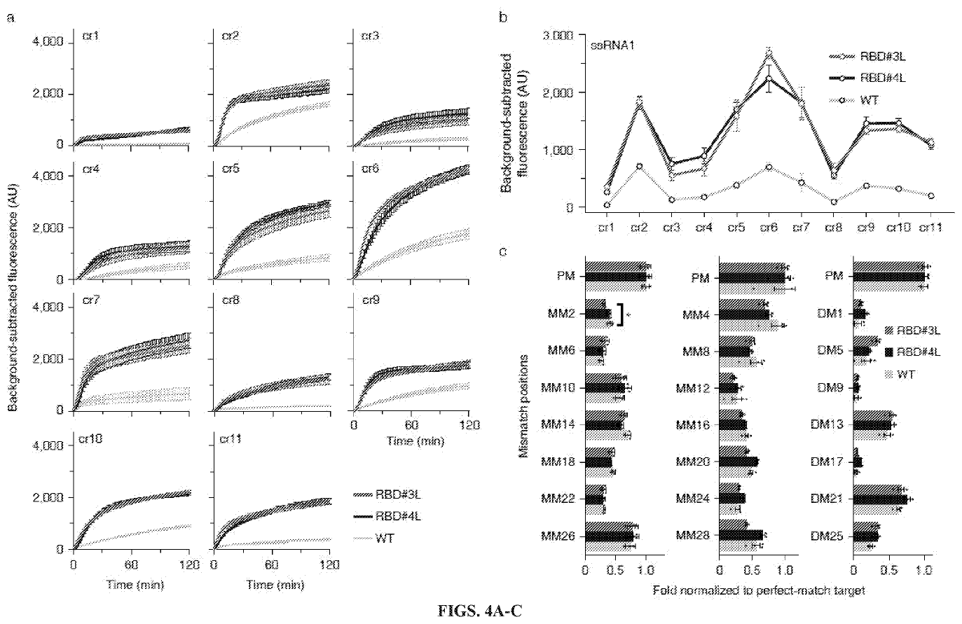

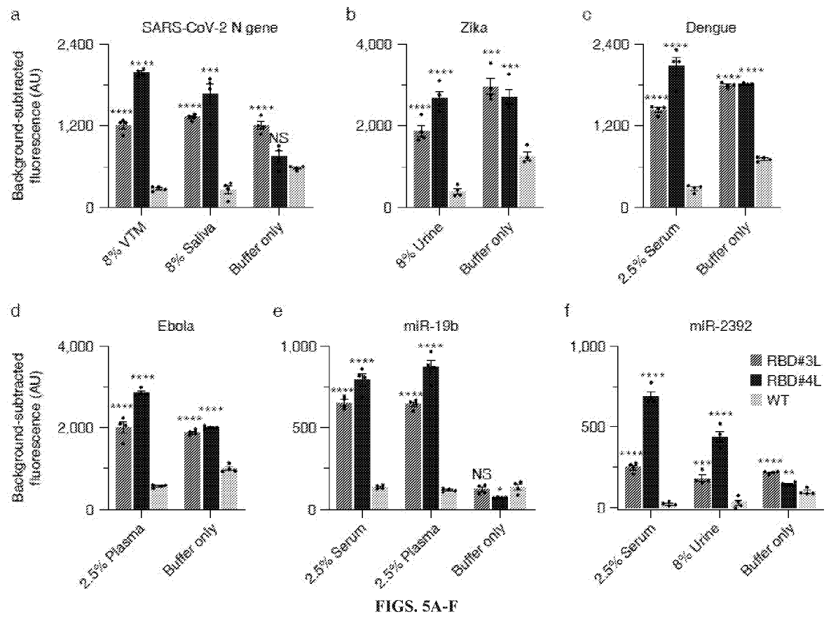

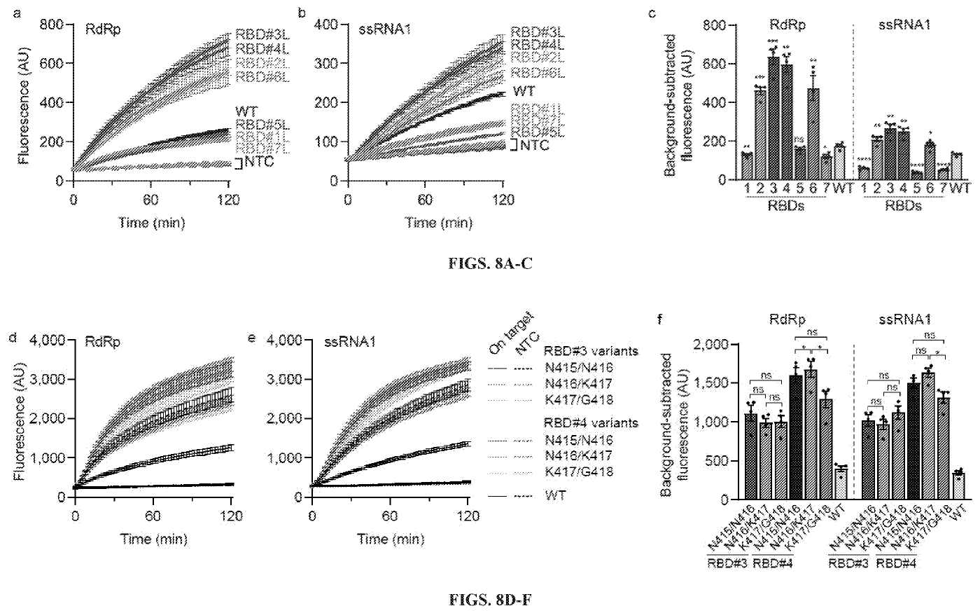

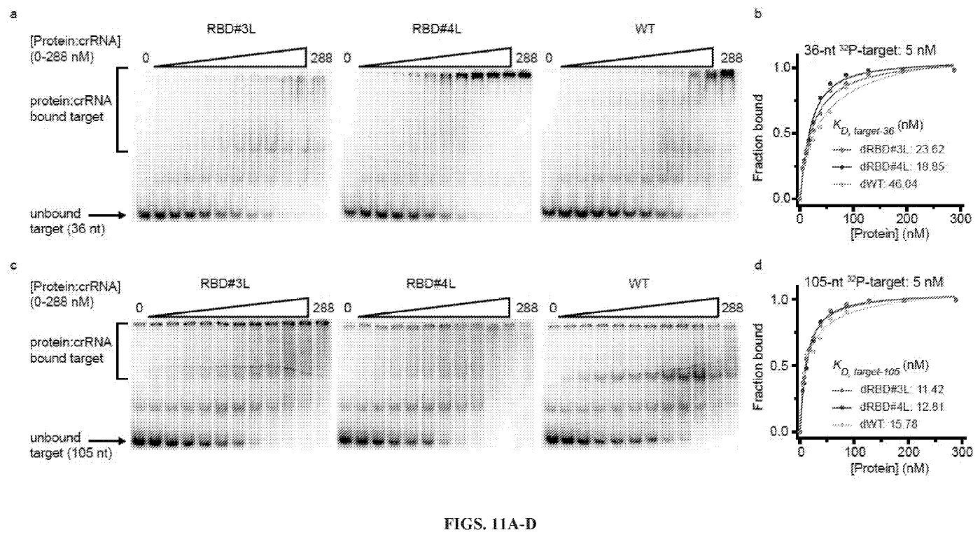

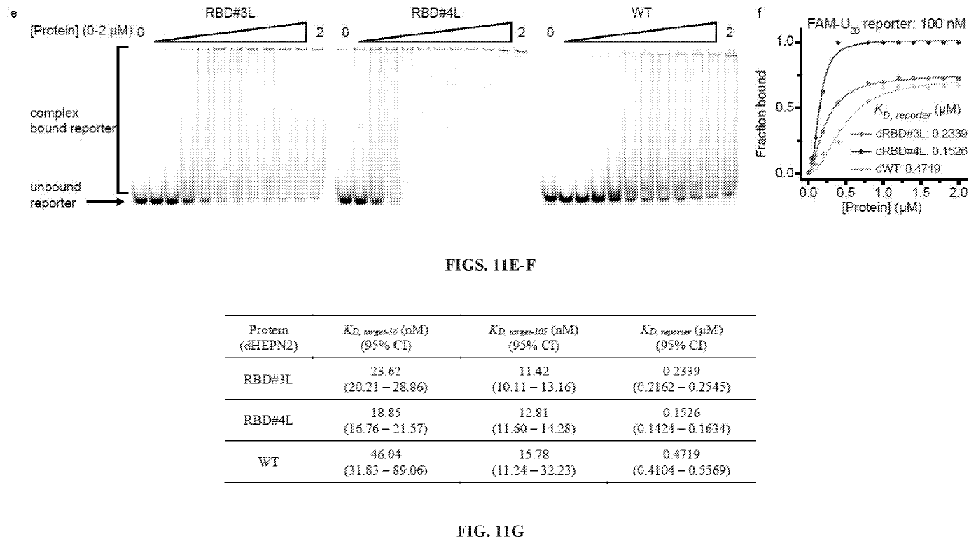

TABLE 1
Target RNA sequences

| Target ID | Sequence (5′→3′) | Description | Related FIGS. |
|---|---|---|---|
| T1_N | Gucugauaauggaccccaaaaucagc | SARS-CoV-2 | FIGS. 1B, 1C, |
| (Fozouni et | gaaaugcaccccgcauuacguuuggu | (Ref Seq: | 2D, 2G, 3, 4C, |
| al., 2021) | ggacccucagauucaacuggcaguaa | NC_045512.2) N | 5A, 6A-6E; 7B, |
| | ccagaauggagaacgcaguggggcgc | gene fragment: | 7C, 9, 16, 13A, |
| | g (SEQ ID NO: 33) | 28,276-28,380 | 13B, and 23 |
| T2_RdRp | Gugauagagccaugccuaacaugcuu | SARS-CoV-2 | FIGS. 8A and |
| (Guo et al., | agaauuauggccucacuuguucuugc | (Ref Seq: | 8D |
| 2020) | ucgcaaacauacaacguguuguagcu | NC_045512.2) | |
| | ugucacaccguuucuauagauuagcu | RdRp gene | |
| | aaugagugugcucaaguauugaguga | fragment: | |
| | aauggucauguguggcgguucacuau | 15,305-15,531 | |
| | auguuaaaccagguggaaccucauca | | |
| | ggagaugccacaacugcuuaugcuaa | | |
| | uaguguuuuuaacauuuu (SEQ | | |
| | ID NO: 34) | | |
| T3_ssRNA1 | Ggccagugaauucgagcucgguaccc | Artificial | FIGS. 8B and |
| (Gootenberg | ggggauccucuagaaauauggauuac | sequence | 8E |
| et al., 2017) | uugguagaacagcaaucuacucgacc | | |
| | ugcaggcaugcaagcuuggcguaauc | | |
| | auggucauagcuguuuccuguguuua | | |
| | uccgcucacaauuccacacaacauac | | |
| | gagccggaagcauaaag (SEQ ID | | |
| | NO: 35) | | |
| T4_N_PM | cgcauuacguuugguggacccucaga | SARS-CoV-2 | FIGS. 4C and |
| | uu (SEQ ID NO: 36) | (Ref Seq: | 19B |
| | | NC_045512.2) N | |
| | | gene fragment: | |
| | | 28,313-28,340 | |
| T5_N_MM2 | cccauuacguuugguggacccucaga | single mismatch | FIGS. 4C and |
| | uu (SEQ ID NO: 37) | at 2^{nd} position | 19B |
| T6_N_MM6 | cgcauaacguuugguggacccucaga | single mismatch | FIGS. 4C and |
| | uu (SEQ ID NO: 38) | at 6^{th} position | 19B |
| T7_N_MM10 | cacauuacgcuugguggacccucaga | single mismatch | FIGS. 4C and |
| | uu (SEQ ID NO: 39) | at 10^{th} position | 19B |
| T8_N_MM14 | cgcauuacguuugcuggacccucaga | single mismatch | FIGS. 4C and |
| | uu (SEQ ID NO: 40) | at 14^{th} position | 19B |
| T9_N_MM18 | cgcauuacguuugguggucccucaga | single mismatch | FIGS. 4C and |
| | uu (SEQ ID NO: 41) | at 18^{th} position | 19B |
| T10_N_MM22 | cgcauuacguuugguggacccacaga | single mismatch | FIGS. 4C and |
| | uu (SEQ ID NO: 42) | at 22^{nd} position | 19B |
| T11_N_MM26 | cgcauuacguuugguggacccucagu | single mismatch | FIGS. 4C and |
| | uu (SEQ ID NO: 43) | at 26^{th} position | 19B |
| T12_N_MM4 | cgcuuuacguuugguggacccucaga | single mismatch | FIGS. 4C and |
| | uu (SEQ ID NO: 126) | at 4^{th} position | 19B |
| T13_N_MM8 | cgcauuagguuugguggacccucaga | single mismatch | FIGS. 4C and |
| | uu (SEQ ID NO: 127) | at 8^{th} position | 19B |
| T14_N_MM12 | cgcauuacguuagguggacccucaga | single mismatch | FIGS. 4C and |
| | uu (SEQ ID NO: 128) | at 12^{th} position | 19B |
| T15_N_MM16 | cacauuacguuugguegacccucaga | single mismatch | FIGS. 4C and |
| | uu (SEQ ID NO: 129) | at 16^{th} position | 19B |
| T16_N_MM20 | cgcauuacguuugguggacgcucaga | single mismatch | FIGS. 4C and |
| | uu (SEQ ID NO: 130) | at 20^{th} position | 19B |
| T17_N_MM24 | cgcauuacguuugguggacccucuga | single mismatch | FIGS. 4C and |
| | uu (SEQ ID NO: 131) | at 24^{th} position | 19B |
| T18_N_MM28 | cacauuacguuugguggacccucaga | single mismatch | FIGS. 4C and |
| | ua (SEQ ID NO: 132) | at 28^{th} position | 19B |
| T19_N_DM1 | gccauuacguuugguggacccucaga | consecutive | FIGS. 4C and |
| | uu (SEQ ID NO: 44) | double mismatch | 19B |
| | | at 1^{st} and 2^{nd} | |
| | | positions | |
| T20_N_DM5 | cgcaaaacguuugguggacccucaga | consecutive | FIGS. 4C and |
| | uu (SEQ ID NO: 45) | double mismatch | |
| | | at 5^{th} and 6^{th} | 19B |
| | | positions | |
| T21_N_DM9 | cgcauuaccauugguggacccucaga | consecutive | FIGS. 4C and |
| | uu (SEQ ID NO: 46) | double mismatch | 19B |
| | | at 9^{th} and 10^{th} | |
| | | positions | |
| T22_N_DM13 | cgcauuacguuuccuggacccucaga | consecutive | FIGS. 4C and |
| | uu (SEQ ID NO: 47) | double mismatch | 19B |
| | | at 13^{th} and 14^{th} | |
| | | positions | |
| T23_N_DM17 | cgcauuacguuuggugcucccucaga | consecutive | FIGS. 4C and |
| | uu (SEQ ID NO: 48) | double mismatch | 19B |
| | | at 17^{th} and 18^{th} | |
| | | positions | |
| T24_N_DM21 | cgcauuacguuugguggaccgacaga | consecutive | FIGS. 4C and |
| | uu (SEQ ID NO: 49) | double mismatch | 19B |
| | | at 21^{st} and 22^{nd} | |
| | | positions | |
| T25_N_DM25 | cacauuacguuugguggacccucacu | consecutive | FIGS. 4C and |
| | uu (SEQ ID NO: 50) | double mismatch | 19B |
| | | at 25^{th} and 26^{th} | |
| | | positions | |
| T26_N_2 | accccgcauuacguuugguggacccu | SARS-CoV-2 | FIGS. 11A-B |
| | cagauucaac (SEQ ID NO: | (Ref Seq: | |
| | 133) | NC_045512.2) N | |
| | | gene fragment: | |
| | | 28,309-28,344 | |
| T27_Zika | agcuggaguguuguuugguaugggca | Zika virus (Ref | FIGS. 5B and |
| (Gootenberg | aagggaugccauucuacgcaugggac | Seq: | 13C |
| et al., 2017) | uuuggagucccgcugcuaaugauagg | NC_035889.1) | |
| | uugcuacucacaauuaacaccccuga | POLY gene | |
| | cccuaauaguggccaucauuuugcuc | fragment: 7,133- | |
| | guggcgcacuacauguacuugauccc | 7,466 | |
| | agggcugcaggcagcagcugcgcgug | | |
| | cugcccagaagagaacggcagcuggc | | |
| | aucaugaagaacccuguuguggaugg | | |
| | aauaguggugacugacauugacacaa | | |
| | ugacaauugacccccaaguggagaaa | | |
| | aagaugggacaggugcuacucauagc | | |
| | aguagcagucuccagcgccaua | | |
| | (SEQ ID NO: 51) | | |
| T28_Dengue | aguacauauucaggggccaaccucuc | Dengue virus | FIGS. 5C and |
| (Gootenberg | aacaaugacgaagaccaugcucacug | type 3 strain | 13D |
| et al., 2017) | gacagaagcaaaaaugcugcuggaca | CH53489 | |
| | acaucaacacaccagaagggauuaua | (GenBank: | |
| | ccagcucucuuugaaccagaaaggga | DQ863638.1) | |
| | gaagucagccgccauagacggugaau | POLY gene | |
| | accgccugaagggu (SEQ ID NO: | fragment: 5,928- | |
| | 52) | 6,097 | |
| T29_Ebola | gaggaaaauaacauugcaaaggugga | Ebola virus (Ref | FIGS. SD and |
| (Barnes et | gccuaugguuuaguuaucuugaucau | seq: | 13E |
| al., 2020) | ugugauaauauccuggcggaggcuuu | NC_002549.1) L | |
| | aacccaaauaacuugcacaguugauu | gene fragment: | |
| | uagcacagauucugagggaauauuca | 14,802-14,974 | |
| | ugggcucauauuuuagagggaagacc | | |
| | ucuuauuggagccacac (SEQ ID | | |
| | NO: 53) | | |
| T30_miR19b | ugugcaaauccaugcaaaacuga | ID: hsa-miR- | FIGS. 5E and |
| | (SEQ ID NO: 54) | 19b-3p | 13F |
| | | (Accession: | |
| | | MIMAT0000074) | |
| T31_miR2392 | uaggaugggggugagaggug (SEQ | ID: hsa-miR- | FIGS. 5F and |
| | ID NO: 55) | 2392 (Accession: | 13G |
| | | MIMAT0019043 | |

Note:
Protospacers are underlined; Mismatches are italicized.
T1-T29 were obtained by IVT and gel-purified (T1-T3 and T26-T29) or magnetic bead-purified (T4-T25).
T30 and T31 were ordered from IDT.

TABLE 2
CRISPR RNA (crRNA) sequences

| | | | Related |
|---|---|---|---|
| crRNA ID | Sequence (5′→3′) | Description | Figures |
| cr1_N | GACUACCCCAAAAACGAAGGGGACUA | targeting T1_N | FIGS. 1B, IC, |
| (Fozouni et | AAACaaucugaggguccaccaaacgu | | 2D, 2G, 3, 4C, |
| al., 2021) | aaugcg (SEQ ID NO: 56) | | 5A, 6A-6E, 7B, |
| | | | 7C, 9, 16, 19B, |
| | | | 13A, 13B, and |
| | | | 23 |
| cr2_RdRp | GACTACCCCAAAAACGAAGGGGACTA | targeting | FIGS. 8A and |
| (Guo et al., | AAACtcactcaatacttgagcacact | T3_RdRp | 8D |
| 2020) | cattag (SEQ ID NO: 57) | | |
| cr3_ssRNA1_ | GACUACCCCAAAAACGAAGGGGACUA | targeting | FIGS. 8B and |
| 0 | AAACuagauugcuguucuaccaagua | T2_ssRNA1 | 8E |
| (Gootenberg | auccau (SEQ ID NO: 58) | | |
| et al., 2017) | | | |
| cr4_ssRNA1_ | GACUACCCCAAAAACGAAGGGGACUA | targeting | FIGS. 4A, 4B, |
| 1 | AAACccggguaccgagcucgaauuca | T2_ssRNA1 | and 19A |
| (Gootenberg | cuggcc (SEQ ID NO: 59) | | |
| et al., 2018) | | | |
| cr5_ssRNA1_ | GACUACCCCAAAAACGAAGGGGACUA | targeting | FIGS. 4A, 4B, |
| 2 | AAACuuucuagaggauccccggguac | T2_ssRNA1 | and 19A |
| (Gootenberg | cgagcu (SEQ ID NO: 60) | | |
| et al., 2018) | | | |
| cr6_ssRNA1_ | GACUACCCCAAAAACGAAGGGGACUA | targeting | FIGS. 4A, 4B, |
| 3 | AAACccaaguaauccauauuucuaga | T2_ssRNA1 | and 19A |
| (Gootenberg | ggaucc (SEQ ID NO: 61) | | |
| et al., 2018) | | | |
| cr7_ssRNA1_ | GACUACCCCAAAAACGAAGGGGACUA | targeting | FIGS. 4A, 4B, |
| 4 | AAACagauugcuguucuaccaaguaa | T2_ssRNA1 | and 19A |
| (Gootenberg | uccaua (SEQ ID NO: 62) | | |
| et al., 2018) | | | |
| cr8_ssRNA1_ | GACUACCCCAAAAACGAAGGGGACUA | targeting | FIGS. 4A, 4B, |
| 5 | AAACccugcaggucgaguagauugcu | T2_ssRNA1 | and 19A |
| (Gootenberg | guucua (SEQ ID NO: 63) | | |
| et al., 2018) | | | |
| cr9_ssRNA1_ | GACUACCCCAAAAACGAAGGGGACUA | targeting | FIGS. 4A, 4B, |
| 6 | AAACgccaagcuugcaugccugcagg | T2_ssRNA1 | and 19A |
| (Gootenberg | ucgagu (SEQ ID NO: 64) | | |
| et al., 2018) | | | |
| cr10_ssRNA1_ | GACUACCCCAAAAACGAAGGGGACUA | targeting | FIGS. 4A, 4B, |
| 7 | AAACaugaccaugauuacgccaagcu | T2_ssRNA1 | and 19A |
| (Gootenberg | ugcaug (SEQ ID NO: 65) | | |
| et al., 2018) | | | |
| cr11_ssRNA1_ | GACUACCCCAAAAACGAAGGGGACUA | targeting | FIGS. 4A, 4B, |
| 8 | AAACcacaggaaacagcuaugaccau | T2_ssRNA1 | and 19A |
| (Gootenberg | gauuac (SEQ ID NO: 66) | | |
| et al., 2018) | | | |
| cr12_ssRNA1_ | GACUACCCCAAAAACGAAGGGGACUA | targeting | FIGS. 4A, 4B, |
| 9 | AAACugugagcggauaaacacaggaa | T2_ssRNA1 | and 19A |
| (Gootenberg | acagcu (SEQ ID NO: 67) | | |
| et al., 2018) | | | |
| cr13_ssRNA1_ | GACUACCCCAAAAACGAAGGGGACUA | targeting | FIGS. 4A, 4B, |
| 10 | AAACauguuguguggaauugugagcg | T2_ssRNA1 | and 19A |
| (Gootenberg | gauaaa (SEQ ID NO: 68) | | |
| et al., 2018) | | | |
| cr14_ssRNA1_ | GACUACCCCAAAAACGAAGGGGACUA | targeting | FIGS. 4A, 4B, |
| 11 | AAACugcuuccggcucguauguugug | T2_ssRNA1 | and 19A |
| (Gootenberg | uggaau (SEQ ID NO: 69) | | |
| et al., 2018) | | | |
| cr15_Zika | GACUACCCCAAAAACGAAGGGGACUA | targeting | FIGS. 5B and |
| (Gootenberg | AAACcauguagugcgccacgagcaaa | T27_Zika | 13C |
| et al., 2017) | augaug (SEQ ID NO: 70) | | |
| cr16_Dengue | GACUACCCCAAAAACGAAGGGGACUA | targeting | FIGS. 5C and |
| (Gootenberg | AAACgguauaaucccuucuggugugu | T28_Dengue | 13D |
| et al., 2017) | ugaugu (SEQ ID NO: 71) | | |
| cr17_Ebola | GACUACCCCAAAAACGAAGGGGACUA | targeting | FIGS. 5D and |
| (Barnes et | AAACaucaacugugcaaguauuugg | T29_Ebola | 13E |
| al., 2020) | guuaaa (SEQ ID NO: 72) | | |
| cr18_miR19b | GACUACCCCAAAAACGAAGGGGACUA | targeting | FIGS. 5E and |
| | AAACucaguuuugcauggauuugcac | T30_miR19b | 13F |
| | a (SEQ ID NO: 73) | | |
| cr19_ | GACUACCCCAAAAACGAAGGGGACUA | targeting | FIGS. 5F and |
| miR2392 | AAACcaccucucacccccauccua | T31_miR2392 | 13G |
| | (SEQ ID NO: 74) | | |

Note:
5′ Direct repeat is in upper case, and spacer is in lower case.
cr1-cr3 were ordered from Synthego.
cr4-cr1929 were obtained by IVT and gel-purified.

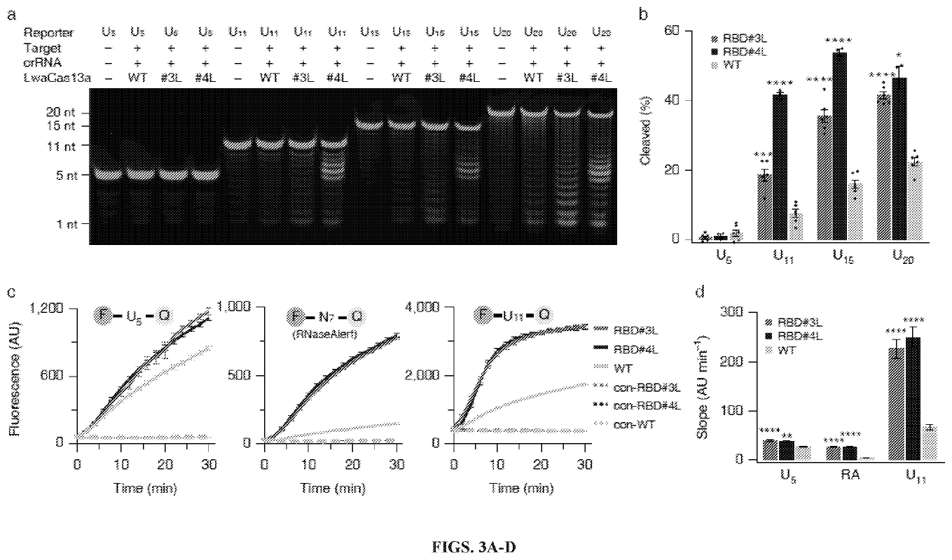

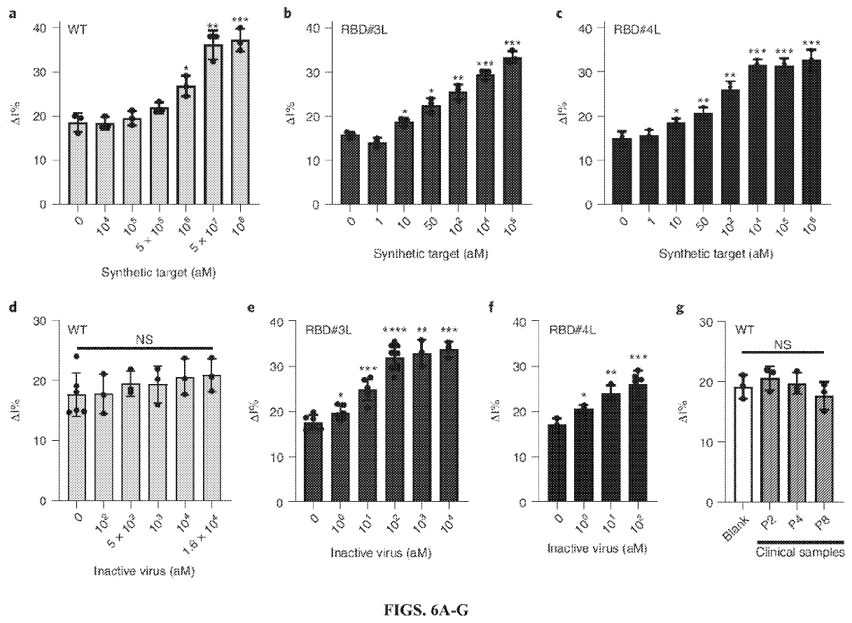

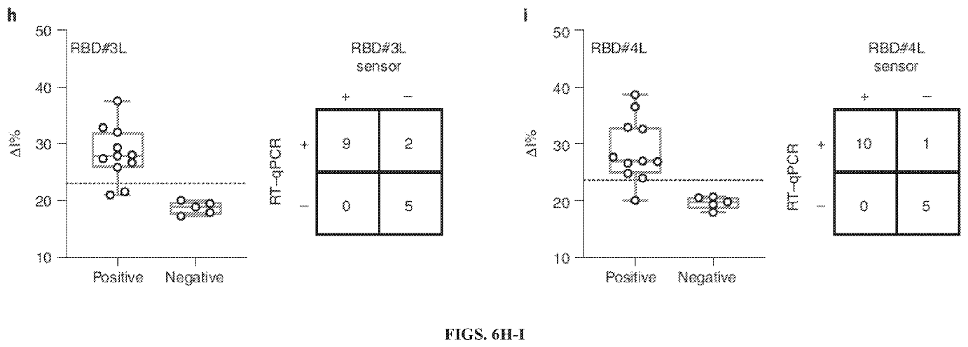

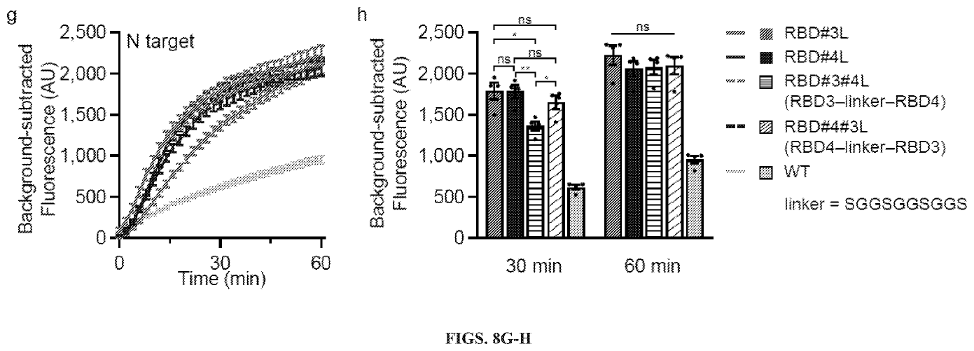

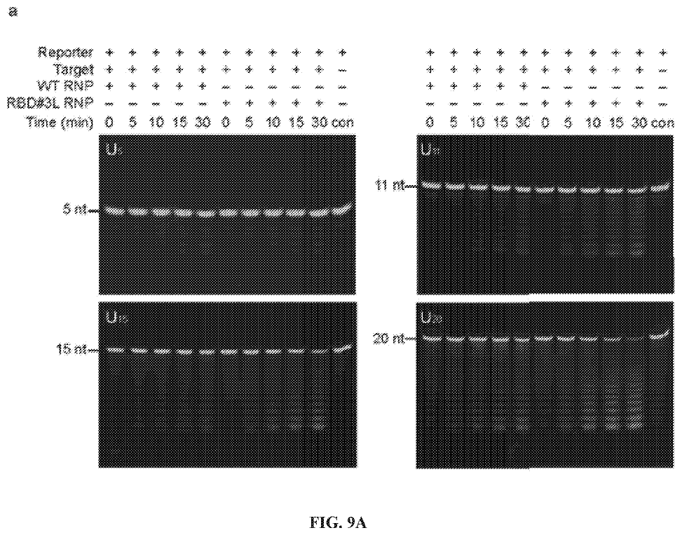

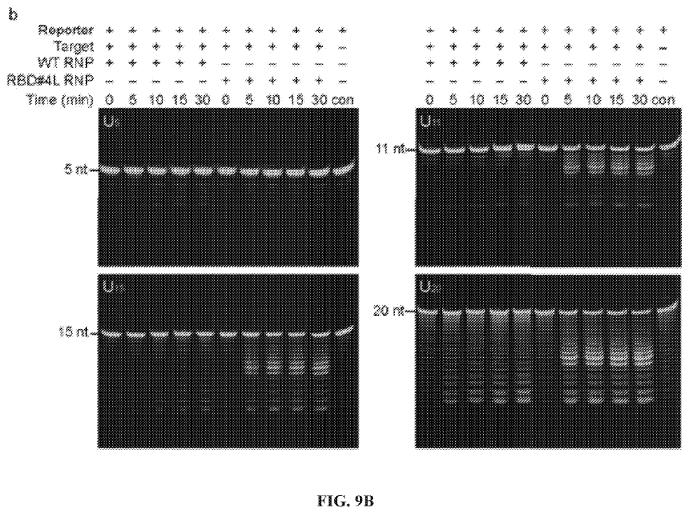

TABLE 3
Reporter RNA oligonucleotide sequences

| reporter | | | |
|---|---|---|---|
| ID | Sequence (5′→3′) | Source | Related Figures |
| r1_FU5Q | /56- | Ordered oligos | FIGS. 3C and 3D |
| | FAM/rUrUrUrUrU/3IABKFQ/ | (IDT) | |
| | (SEQ ID NO: 75) | | |
| r2_FU5 | /56-FAM/rUrUrUrUrU | Ordered oligos | FIGS. 3A, 3B, and 9 |
| | (SEQ ID NO: 75) | (IDT) | |
| r3_FU11Q | /56-FAM/ | Ordered oligos | FIGS. 3C, 3D, 4, 5, 8, 13, |
| | rUrUrUrUrUrUrUrUrUrUrU | (IDT) | 16, and 19 |
| | /3IABKFQ/ (SEQ ID NO: | | |
| | 76) | | |
| r4_FU11 | /56- | Ordered oligos | FIGS. 3A, 3B, and 9 |
| | FAM/rUrUrUrUrUrUrUrUrU | (IDT) | |
| | rUrU (SEQ ID NO: 76) | | |
| r5_FU15 | /56-FAM/ | Ordered oligos | FIGS. 3A, 3B, and 9 |
| | rUrUrUrUrUrUrUrUrUrUrU | (IDT) | |
| | rUrUrUrU (SEQ ID NO: | | |
| | 77) | | |
| r6_FU20 | /56-FAM/ | Ordered oligos | FIGS. 3A, 3B, and 9 |
| | rUrUrUrUrUrUrUrUrUrUrU | (IDT) | |
| | rUrUrUrUrUrUrUrUrU | | |
| | (SEQ ID NO: 78) | | |
| r7_MBU20 | /5ThioMC6-D/ | Ordered oligos | FIGS. 6B-6F, 14, and 23 |
| | rUrUrUrUrUrUrUrUrUrUrU | (IDT) | |
| | rUrUrUrUrUrUrUrUrU/3Me | | |
| | BIN/ (SEQ ID NO: 78) | | |
| RNaseAlert | (N)₇ | IDT | FIGS. 1B, 1C, 2D, 2G, |
| | | | 3C, and 3D |

Notes: /56-FAM/:
5′ 6-FAM (carboxyfluorescein); /3IABKFQ/: 3′ Iowa Black quencher; /5ThioMC6-D/: 5′ Thiol Modifier C6 S-S modification; /3MeBIN/: 3′ Methylene blue.

TABLE 4
IVT Primers

| Primer ID | Sequences (5′→3′) |
|---|---|
| Pr01_cr_univ_F | GAAATTAATACGACT |
| | CACTATAGGGACTAC |
| | CCCAAAAACGAAGGG |
| | GACTAAAAC |
| | (SEQ ID NO: 79) |
| Pr02_cr4_ssRNA1_R1 | GGCCAGTGAATTCGA |
| | GCTCGGTACCCGGGT |
| | TTTAGTCCCCTTCGT |
| | TTTTGGGGT |
| | (SEQ ID NO: 80) |
| Pr03_cr5_ssRNA1_R2 | AGCTCGGTACCCGGG |
| | GATCCTCTAGAAAGT |
| | TTTAGTCCCCTTCGT |
| | TTTTGGGGT |
| | (SEQ ID NO: 81) |
| Pr04_cr6_ssRNA1_R3 | GGATCCTCTAGAAAT |
| | ATGGATTACTTGGGT |
| | TTTAGTCCCCTTCGT |
| | TTTTGGGGT |
| | (SEQ ID NO: 82) |
| Pr05_cr7_ssRNA1_R4 | TATGGATTACTTGGT |
| | AGAACAGCAATCTGT |
| | TTTAGTCCCCTTCGT |
| | TTTTGGGGT |
| | (SEQ ID NO: 83) |
| Pr06_cr8_ssRNA1_R5 | TAGAACAGCAATCTA |
| | CTCGACCTGCAGGGT |
| | TTTAGTCCCCTTCGT |
| | TTTTGGGGT |
| | (SEQ ID NO: 84) |
| Pr07_cr9_ssRNA1_R6 | ACTCGACCTGCAGGC |
| | ATGCAAGCTTGGCGT |
| | TTTAGTCCCCTTCGT |
| | TTTTGGGGT |
| | (SEQ ID NO: 85) |
| Pr08_cr10_ssRNA1_R7 | CATGCAAGCTTGGCG |
| | TAATCATGGTCATGT |
| | TTTAGTCCCCTTCGT |
| | TTTTGGGGT |
| | (SEQ ID NO: 86) |
| Pr09_cr11_ssRNA1_R8 | GTAATCATGGTCATA |
| | GCTGTTTCCTGTGGT |
| | TTTAGTCCCCTTCGT |
| | TTTTGGGGT |
| | (SEQ ID NO: 87) |
| Pr10_cr12_ssRNA1_R9 | AGCTGTTTCCTGTGT |
| | TTATCCGCTCACAGT |
| | TTTAGTCCCCTTCGT |
| | TTTTGGGGT |
| | (SEQ ID NO: 88) |
| Pr11_cr13_ssRNA1_R10 | TTTATCCGCTCACAA |
| | TTCCACACAACATGT |
| | TTTAGTCCCCTTCGT |
| | TTTTGGGGT |
| | (SEQ ID NO: 89) |
| Pr12_cr14_ssRNA1_R11 | ATTCCACACAACATA |
| | CGAGCCGGAAGCAGT |
| | TTTAGTCCCCTTCGT |
| | TTTTGGGGT |
| | (SEQ ID NO: 90) |
| Pr13_cr15_Zika_R | CATCATTTTGCTCGT |
| | GGCGCACTACATGGT |
| | TTTAGTCCCCTTCGT |
| | TTTTGGGGT |
| | (SEQ ID NO: 91) |
| Pr14_cr16_Dengue_R | ACATCAACACACCAG |
| | AAGGGATTATACCGT |
| | TTTAGTCCCCTTCGT |
| | TTTTGGGGT |
| | (SEQ ID NO: 92) |
| Pr15_cr17_Ebola_R | TTTAACCCAAATAAC |
| | TTGCACAGTTGATGI |
| | TTTAGTCCCCTTCGT |
| | TTTTGGGGT |
| | (SEQ ID NO: 93) |
| Pr16_cr18_miR19b_R | TGTGCAAATCCATGC |
| | AAAACTGAGTTTTAG |
| | TCCCCTTCGTTTTTG |
| | GGGT |
| | (SEQ ID NO: 94) |
| Pr17_cr19_miR2392_R | TAGGATGGGGGTGAG |
| | AGGTGGTTTTAGTCC |
| | CCTTCGTTTTTGGGG |
| | T |
| | (SEQ ID NO: 95) |
| Pr18_Tar_3G_F | GAAATTAATACGACT |
| | CACTATAGGG |
| | (SEQ ID NO: 96) |
| Pr19_TI_N_R | CGCGCCCCACTGCGT |
| | TCTCC |
| | (SEQ ID NO: 97) |
| Pr20_T2_ssRNA1_R | CTTTATGCTTCCGGC |
| | TCGTA |
| | (SEQ ID NO: 98) |
| Pr21_T3_RdRp_R | ACAAATGTTAAAAAC |
| | ACTATTAGCATAA |
| | (SEQ ID NO: 99) |
| Pr22_T4-18_T20-25_F | GAAATTAATACGACT |
| | CACTATAGGC |
| | (SEQ ID NO: 100) |
| Pr23_T4_R | AATCTGAGGGTCCAC |
| | CAAACGTAATGCGCC |
| | TATAGTGAGTCGTAT |
| | TAATTTC |
| | (SEQ ID NO: 101) |
| Pr24_T5_R | AATCTGAGGGTCCAC |
| | CAAACGTAATGGGCC |
| | TATAGTGAGTCGTAT |
| | TAATTTC |
| | (SEQ ID NO: 102) |
| Pr25_T6_R | AATCTGAGGGTCCAC |
| | CAAACGTTATGCGCC |
| | TATAGTGAGTCGTAT |
| | TAATTTC |
| | (SEQ ID NO: 103) |
| Pr26_T7_R | AATCTGAGGGTCCAC |
| | CAAGCGTAATGCGCC |
| | TATAGTGAGTCGTAT |
| | TAATTTC |
| | (SEQ ID NO: 104) |
| Pr27_T8_R | AATCTGAGGGTCCAG |
| | CAAACGTAATGCGCC |
| | TATAGTGAGTCGTAT |
| | TAATTTC |
| | (SEQ ID NO: 105) |
| Pr28_T9_R | AATCTGAGGGACCAC |
| | CAAACGTAATGCGCC |
| | TATAGTGAGTCGTAT |
| | TAATTTC |
| | (SEQ ID NO: 106) |
| Pr29_T10_R | AATCTGTGGGTCCAC |
| | CAAACGTAATGCGCC |
| | TATAGTGAGTCGTAT |
| | TAATTTC |
| | (SEQ ID NO: 107) |
| Pr30_T11_R | AAACTGAGGGTCCAC |
| | CAAACGTAATGCGCC |
| | TATAGTGAGTCGTAT |
| | TAATTTC |
| | (SEQ ID NO: 108) |
| Pr31_T12_R | AATCTGAGGGTCCAC |
| | CAAACGTAATGGCCC |
| | TATAGTGAGTCGTAT |
| | TAATTTC |
| | (SEQ ID NO: 109) |
| Pr32_T13_R | AATCTGAGGGTCCAC |
| | CAAACGTTTTGCGCC |
| | TATAGTGAGTCGTAT |
| | TAATTTC |
| | (SEQ ID NO: 110) |
| Pr33_T14_R | AATCTGAGGGTCCAC |
| | CAATGGTAATGCGCC |
| | TATAGTGAGTCGTAT |
| | TAATTTC |
| | (SEQ ID NO: 111) |
| Pr34_T15_R | AATCTGAGGGTCCAG |
| | GAAACGTAATGCGCC |
| | TATAGTGAGTCGTAT |
| | TAATTTC |
| | (SEQ ID NO: 112) |
| Pr35_T16_R | AATCTGAGGGAGCAC |
| | CAAACGTAATGCGCC |
| | TATAGTGAGTCGTAT |
| | TAATTTC |
| | (SEQ ID NO: 113) |
| Pr36_T17_R | AATCAGAGGGTCCAC |
| | CAAACGTAATGCGCC |
| | TATAGTGAGTCGTAT |
| | TAATTTC |
| | (SEQ ID NO: 134) |
| Pr37_T18_R | TATCTGAGGGTCCAC |
| | CAAACGTAATGCGCC |
| | TATAGTGAGTCGTAT |
| | TAATTTC |
| | (SEQ ID NO: 135) |
| Pr38_T19_R | AATCTGAGGGTCCAC |
| | CAAACGTAATGGCCC |
| | TATAGTGAGTCGTAT |
| | TAATTTC |
| | (SEQ ID NO: 136) |
| Pr39_T20_R | AATCTGAGGGTCCAC |
| | CAAACGTTTTGCGCC |
| | TATAGTGAGTCGTAT |
| | TAATTTC |
| | (SEQ ID NO: 137) |
| Pr40_T21_R | AATCTGAGGGTCCAC |
| | CAATGGTAATGCGCC |
| | TATAGTGAGTCGTAT |
| | TAATTTC |
| | (SEQ ID NO: 138) |
| Pr41_T22_R | AATCTGAGGGTCCAG |
| | GAAACGTAATGCGCC |
| | TATAGTGAGTCGTAT |
| | TAATTTC |
| | (SEQ ID NO: 139) |
| Pr42_T23_R | AATCTGAGGGAGCAC |
| | CAAACGTAATGCGCC |
| | TATAGTGAGTCGTAT |
| | TAATTTC |
| | (SEQ ID NO: 140) |
| Pr43_T24_R | AATCTGTCGGTCCAC |
| | CAAACGTAATGCGCC |
| | TATAGTGAGTCGTAT |
| | TAATTTC |
| | (SEQ ID NO: 114) |
| Pr44_T25_R | AAAGTGAGGGTCCAC |
| | CAAACGTAATGCGCC |
| | TATAGTGAGTCGTAT |
| | TAATTTC |
| | (SEQ ID NO: 115) |
| Pr45_T26_N_2_F | GAAATTAATACGACT |
| | CACTATAGGACCCCG |
| | CATTACGTTTGGTGG |
| | ACCCTCAGATTCAAC |
| | (SEQ ID NO: 141) |
| Pr46_T26_N_2_R | GTTGAATCTGAGGGT |
| | CCACCAAACGTAATG |
| | CGGGGTCCTATAGTG |
| | AGTCGTATTAATTTC |
| | (SEQ ID NO: 142) |
| Pr47_T27_Zika_R | TATGGCGCTGGAGAC |
| | TGCTACT |
| | (SEQ ID NO: 116) |
| Pr48_T28_Dengue_F | GAAATTAATACGACT |
| | CACTATAGGGAGTAC |
| | ATATTCAGGGGCCAA |
| | CCT |
| | (SEQ ID NO: 117) |
| Pr49_T28_Dengue_R | ACCCTTCAGGCGGTA |
| | TTCACC |
| | (SEQ ID NO: 118) |
| Pr50_T29_Ebola_F | GAAATTAATACGACT |
| | CACTATAGGGGAGGA |
| | AAATAACATTGCAAA |
| | GGTGGAG |
| | (SEQ ID NO: 119) |
| Pr51_T29_Ebola_R | GTGTGGCTCCAATAA |
| | GAGGTCTTC |
| | (SEQ ID NO: 120) |

Simulation sample preparation. Synthetic targets that spiked into various biofluids were treated by the HUDSON method (Arizti-Sanz et al., 2020; Myhrvold et al., 2018; Barnes et al., 2020; Shan et al., 2019). The VTM (BioVision, Cat. M1515-50, Lot 7F14M15150), pooled human saliva (Innovative Research, IRHUSL5ML-34462), and pooled human urine (Innovative Research, IRHUURE50ML-35650) were supplemented with 1 M TCEP, 500 mM EDTA, and Murine RNase inhibitor (40 U/μL) at a volume ratio of 100:11.39:0.23:2.28 with final concentrations of 100 mM TCEP, 1 mM EDTA, and 0.8 U/μL inhibitor. The following heating steps were performed in a thermocycler (BioRad). Simulation sample preparation details are provided in Table 6.

TABLE 6
Simulation sample preparation

| Targets | Biofluids | Heating steps | Final concentrations | Related figures |
|---|---|---|---|---|
| SARS- | VTM | Incubated at 40° C. for | 10 pM targets, 8% | FIGS. 5A; FIG. |
| CoV-2 | | 5 min, combined with | VTM, and 50 ng | 13A |
| Ngene | | target RNA and bgRNA, | bgRNA | |
| (T1) | | and heated at | 50 pM targets, 16% or | FIGS. 14A |
| | | 70° C. for 5 min | 8% VTM, and 50 ng | |
| | | | bgRNA | |
| | Saliva | Incubated at 40° C. for | 10 pM targets, 8% | FIGS. 5A; FIG. |
| | | 5 min, combined with | saliva, and 50 ng | 13B |
| | | target RNA and | bgRNA | |
| | | bgRNA, and heated at | | |
| | | 95° C. for 5 min | | |
| Zika | Urine | Incubated at 50° C. for | 10 pM targets, 8% urine, | FIGS. 5B and |
| (T27) | | 20 min, combined with | and 50 ng bgRNA | 13C |
| | | target RNA and | 50 pM targets, 16% or | FIGS. 14B |
| | | bgRNA, and heated at | 8% urine, and 50 ng | |
| | | 95° C. for 5 min | bgRNA | |
| Dengue | Serum | Incubated at 42° C. for | 10 pM targets, 2.5% | FIGS. 5C and |
| (T28) | | 20 min, combined with | serum, and 50 ng | 13D |
| | | target RNA and | bgRNA | |
| | | bgRNA, heated at | 50 pM targets, 4.8% or | FIGS. 14C |
| | | 64° C. for 5 min, and | 2.5% serum, and 50 ng | |
| | | spun down shortly to | bgRNA | |
| | | remove any aggregates | | |
| Ebola | Plasma | Incubated at 27° C. for | 10 pM targets, 2.5% | FIGS. 5D and |
| (T29) | | 20 min, combined with | plasma, and 50 ng | 13E |
| | | target RNA and | bgRNA | |
| | | bgRNA, heated at | 50 pM targets, 4.8% or | FIGS. 14D |
| | | 95° C. for 10 min, and | 2.5% plasma, and 50 ng | |
| | | spun down shortly to | bgRNA | |
| | | remove any aggregates | | |
| miR-19b | Serum or | Incubated at 50° C. for | 10 pM targets, 2.5% | FIGS. 5E and |
| (T30) | Plasma | 20 min, combined with | serum or plasma, and | 13F-G |
| | | target RNA and | 50 ng bgRNA | |
| | | bgRNA, | | |
| miR- | Serum | Incubated at 50° C. for | 200 pM targets, 2.5% | FIGS. 5F and |
| 2392 | | 20 min and 65° C. for 5 | serum, and 50 ng | 13H |
| (T31) | | min | bgRNA | |
| | Urine | Incubated at 50° C. for | 200 pM targets, 8% | FIGS. 5F and |
| | | 20 min and 95° C. for 5 | urine, and 50 ng bgRNA | 13I |
| | | min | | |

SARS-Cov-2 heat-inactivated and clinical samples treatment. Heat-inactivated SARS-CoV-2 samples from infected Vero E6 cell lysate and culture supernatant were purchased from ATCC (VR-1986HK™) with a viral load of 3.9×10⁵ copies per microliter (Lot #70042082).

Clinical samples were leftover specimens of nasopharyngeal swabs in VTM from SARS-CoV-2 infected patients or suspects from the Department of Pathology Microbiology laboratory at UConn Health. Nasopharyngeal swabs in VTM were obtained from hospitalized patients at UConn Health or outpatients at the UConn Drive Through COVID Testing Center. After isolation of the viral target, clinical samples were amplified by Transcription Mediated Amplification (TMA) or RT-PCR to detect either the ORF1ab (including pplab), E1 (viral envelope) and/or N2 (nucleocapsid 2) target sequences. Ethical approval of the study was provided by the UConn Health Institutional Review Board. Positive or negative samples as VTM leftovers were inactivated at 60° C. for 30 min before transfer to Zhang Lab at the Institute of Materials Science at UConn.

The heat-inactive cultivated or clinical virus samples were mixed with Quick Extract™ DNA Extraction Solution (Lucigen, QE09050) with a 1:1 volume ratio and incubated at 95° C. for 5 min in a water bath to actively lyse viral particles and inactivate nucleases in samples (Ladha et al., 2020; Ning et al., 2021). The purchased viral samples were serially diluted in water at various concentrations before use. The treated clinical samples were directly used for detection.

Fluorescence plate reader assay. Fluorescence assays were performed as previously described with modifications (Gootenberg et al., 2017). The protein stock was diluted to 450 nM using the storage buffer (50 mM Tris-HCl, 600 mM NaCl, 5% Glycerol, and 2 mM DTT, pH 7.5). The crRNA was diluted to 450 nM using nuclease-free water. Then, the ribonucleoprotein (RNP) was formed by mixing protein and crRNA in a ratio of 2:1 (v:v) at room temperature for 30 min. The 10 μL reactions contained 45 nM WT or RBD-LwaCas13a fusion protein, 22.5 nM crRNA, 125 nM reporter, 1 U/μL Murine RNase Inhibitor (New England Biolabs), 10-50 pM target, and the reaction buffer (50 mM Tris-HCl, 5 mM Mg²⁺, pH 8.0), unless otherwise indicated.

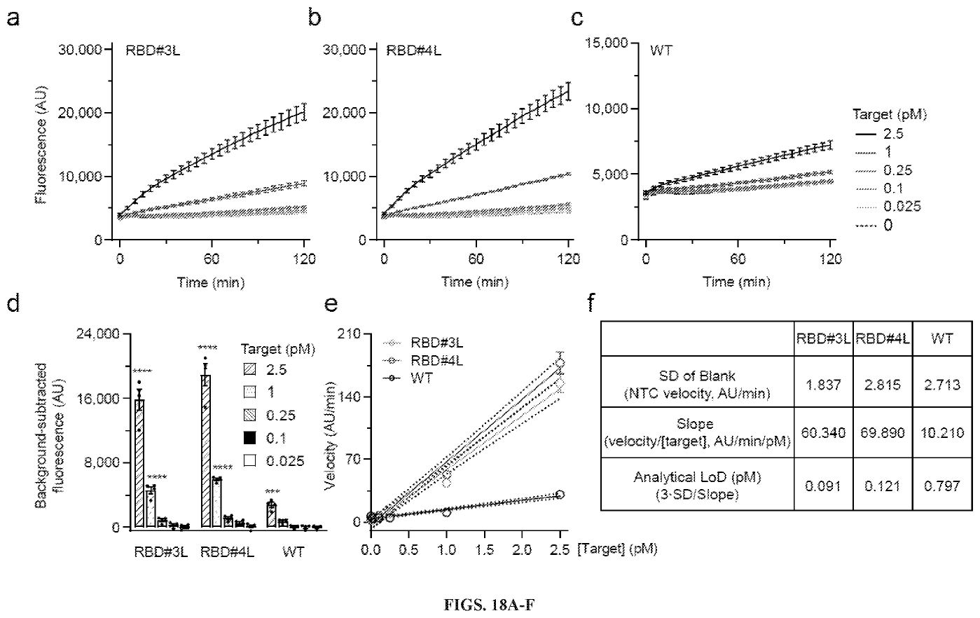

For FIGS. 1B, 1C, 2D, 2G, 3C, 3D, 4, 7B, 7C, 8, 10, and 16-18, the reactions were initiated by adding eight parts of master mix containing RNP, RNase Inhibitor, Tris and Mg²⁺ to two parts of the mixture containing an equal volume of target and reporter. The controls were performed in parallel using same amount of nuclease-free water to replace the targets.

For FIGS. 5, 13, and 14, the reactions were initiated by adding 8 parts of the master mix containing RNP, RNase Inhibitor, reporter, Tris, and Mg²⁺ to 2 parts of the mixture containing target, background RNA and biofluid/water. The controls were performed in parallel using the same amount of nuclease-free water instead of the targets in the presence or absence of the biofluid.

For FIGS. 1B, IC, 2D, 2G, 3C (mid panel), 7B, 7C, and 8, the reporters were RNaseAlert. For FIG. 3C (left panel), the reporters were self-quenched U₅ (SEQ ID NO: 75, r1, Table 3). For FIGS. 3C (right panel), 4, 5, 9, 11, 13, and 14, the reporters were self-quenched U₁₁ (SEQ ID NO: 76, r3, Table 3).

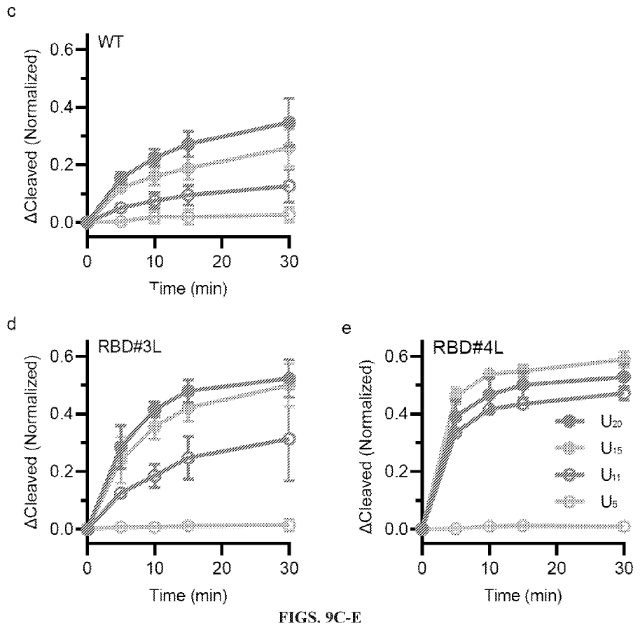

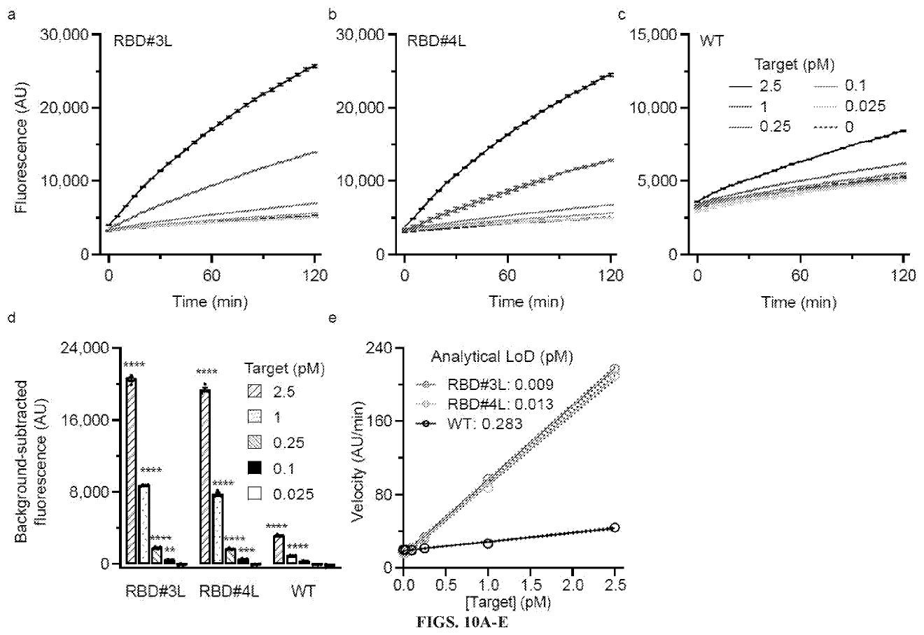

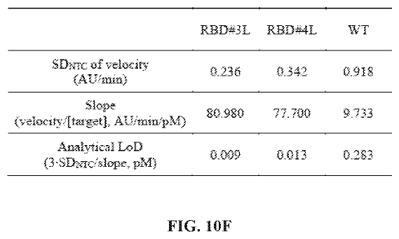

All reactions were performed in a 384-well microplate (Greiner, Cat. 784900) at 37° C., with fluorescence monitored every 2 min over 30-120 min on a TECAN infinity M200 plate reader (Excitation: 490 nm, Emission: 520 nm, gain: 100; or excitation: 485 nm, emission: 528 nM, gain: 150 for reactions in FIGS. 10, 17 and 18).

Fluorescence gel assay. Reactions were carried out as described above in fluorescence plate reader assay, except the fluorophore-quencher reporters were replaced by 5′-FAM-labeled U₅, U₁₁, U₁₅, or U₂₀ (SEQ ID NOS: 75-78, r2, and r4-6, Table 3). A total volume of 110 μL reactions was initiated by adding target RNA (SEQ ID NO: 33, T1) at a final concentration of 10 pM. Aliquots of 20-μL reaction were removed at 0, 5, 10, 15, and 30 min, quenched by mixing with an equal amount of 2×Loading buffer (93.5% Formamide, 0.025% Xylene cyanol FF, and 20 mM EDTA, pH 8.0), incubated at 95° C. for 5 min, and snap-cooled on ice. Quenched reactions were resolved on 22.5% (v/v) denaturing polyacrylamide gel, visualized and captured with a Sapphire Biomolecular Imager (Azure Biosystems). Assays were performed in three technical replicates, and the band intensity of the substrates and products was analyzed using ImageJ. The percent cleavage of each reporter was determined as the ratio of band intensity of all cleavage products to the total intensity within the lane, i.e., the sum of intensity from the cleaved and uncleaved reporter, and normalized for the background of each measured reporter.

Electrophoretic mobility shift assay. To quantify the target binding affinity, assays were carried out in binding buffer (50 mM Tris-HCl, pH 8.0, 60 mM NaCl, 5 mM MgCl₂, 1 mM DTT and 10% glycerol) with a serial dilution of the protein:crRNA complex (protein:crRNA=1:0.95, protein from 5 nM to 288 nM). HEPN2 mutants dRBD #3L, dRBD #4L, and dWT LwaCas13a, were respectively complexed with crRNA ([SEQ ID NO: 56], cr1; Table 2) for 30 min at room temperature, then incubated with 5 nM body-radiolabeled RNA targets (T1 [SEQ ID NO: 33] or T26 [SEQ ID NO: 133]; Table 1) for another 30 min at room temperature, and further incubated at 37° C. for 10 min. Each sample was mixed with a loading buffer containing 10% glycerol and 0.05% bromophenol blue before loading onto a 5% native polyacrylamide gel containing 0.5×TBE buffer. The gel was pre-run at 4° C. for 45 min at 120 V with 0.5×TBE as a running buffer. Gels were dried on filter paper with a HydroTech Pump Gel Drying Complete System (BioRad), followed by exposure to a phosphorimager plate for 48 h, and imaged using phosphorimaging by the Sapphire Biomolecular Imager (Azure Biosystems).

The reporter binding affinity assay was carried out in binding buffer (50 mM Tris-HCl pH 7.5, 60 mM NaCl, 5 mM MgCl₂, 1 mM DTT, 5% glycerol, and 0.01% Triton X-100). The RNP complex was formed by incubating the protein and crRNA at room temperature for 30 min and serially diluted with the binding buffer (protein:crRNA=2:1, protein from 50 nM to 2 μM). To mimic the target-activated protein complex and avoid competitive target binding, an equal amount of 50 pM of the target RNA was added to each of the RNP dilutions and incubated at 37° C. for 30 min. The reactions were further incubated with 100 nM of 5′-FAM-labeled U₂₀ reporter ([SEQ ID NO: 78] r6; Table 3) for 10 min at 37° C. Each sample was mixed with a loading buffer containing 10% glycerol and 0.05% bromophenol blue before loading onto a 6% native polyacrylamide gel containing 1×TG buffer at 4° C. (25 mM Tris base, 250 mM glycine). The gels were pre-run at 4° C. for 45 min at 120 V with 1×TG running buffer and visualized with a Sapphire Biomolecular Imager (Azure Biosystems). All experiments were carried out with two technical replicates. The bound and unbound fraction of target or reporter RNA was quantified by using Azure Spot (Azure Biosystem), plotted in GraphPad Prism, and fitted by non-linear regression with saturation one-site binding.

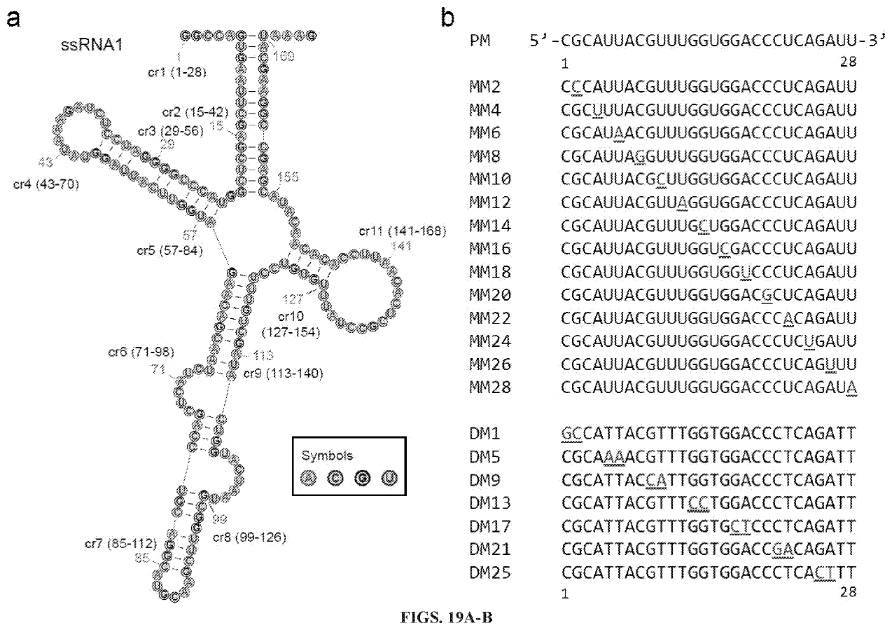

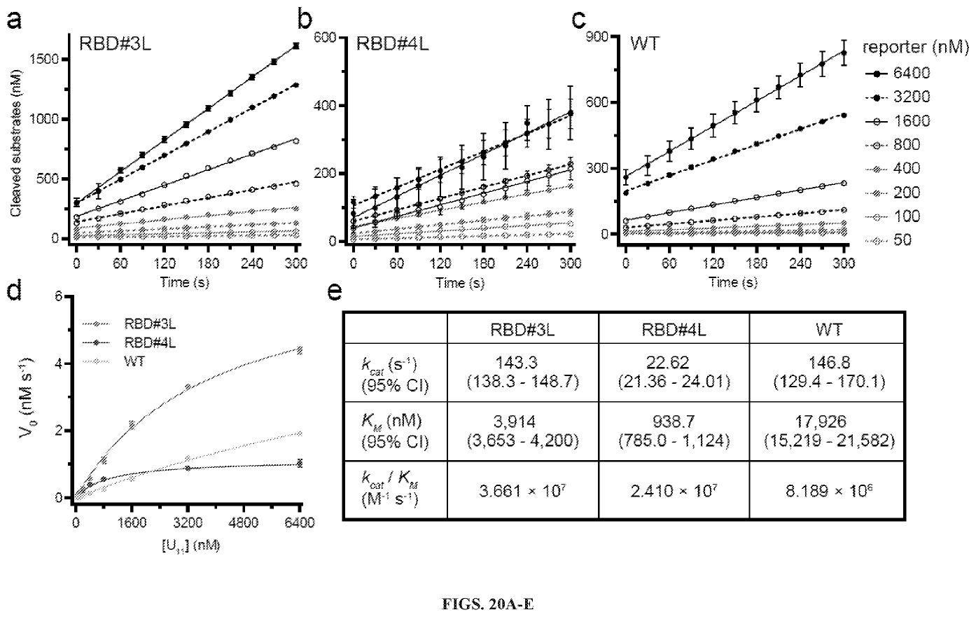

Michaelis-Menton kinetic study. For Michaelis-Menten analysis, reactions were prepared by incubating 45 nM protein (WT/RBD #3L/RBD #4L) with 22.5 nM crRNA (SEQ ID NO: 56, cr1) to form the RNP in reaction buffer at room temperature for 30 min, followed by incubating at 37° C. for 10 min with 50 pM RNA targets (SEQ ID NO: 33, T1). Collateral cleavage was initiated by adding two parts of the substrate (SEQ ID NO: 76, r3; Table 3) at various concentrations, that is, 6400, 3200, 1600, 800, 400, 200, 100, and 50 nM, to eight parts of the target-activated RNP and incubated at 37° C. on an M200 plate reader with fluorescence taken every 30 s for up to 10 min (excitation: 490 nm, emission: 520 nm). Reactions (10 μl) were performed on a 384-well microplate, with four technical replicates from two independent experiments in buffer containing 50 mM Tris, pH 8.0, 5 mM MgCl₂, and 1 U/μl RNase Inhibitor (FIG. 12); or with four technical replicates in buffer containing 50 mM Tris, pH 8.0, 5 mM MgCl₂, 1 U/μl RNase Inhibitor, 100 μg/ml BSA, and 0.01% Triton X-100 (FIG. 20).

To convert the time-course fluorescence signal (F_{[U11]}) to the molarity of cleavage products ([Cleaved U₁₁]), the reporters were serially diluted using the reaction buffers mentioned above, in the presence or absence of 50 μg ml⁻¹ RNaseA (NEB, T3018L), to the same concentrations as in the assay. The level of fluorescence from quenched reporters (no RNaseA, F_{0,[U11]}) and completely cleaved reporters (RNaseA treated, F_{F,[U11]}) at each concentration was recorded for up to 30 min at 37° C. until reaching equilibrium. The molarity of cleavage products at each time point was calculated using the following equation (1):

$$[ Cleaved {U}_{11} ] = \frac{{F}_{[ {U}_{11} ]} - {F}_{0 , [ {U}_{11} ]}}{{F}_{F , [ {U}_{11} ]} - {F}_{0 , [ {U}_{11} ]}} \times [ {U}_{11} ]$$

where [U₁₁]=6400, 3200, 1600, 800, 400, 200, 100, and 50 nM.

Since the cleavage of 1 nM reporter produced similar levels of fluorescence signal with an average ˜25-28 AU nM⁻¹ at all tested reporter concentrations, the above equation (1) was then simplified to equation (2):

$$[ Cleaved {U}_{11} ] = \frac{{F}_{[ {U}_{11} ]} - {F}_{0 , [ {U}_{11} ]}}{Average ( \frac{{F}_{F , [ {U}_{11} ]} - {F}_{0 , [ {U}_{11} ]}}{{U}_{11}} )}$$

The cleaved product molarity versus time was fitted by linear regression (GraphPad). The slope as initial velocity (nM s⁻¹) was plotted as a function of substrate concentration [U₁₁] (nM), and further fitted to the Michaelis-Menten model (GraphPad) to determine kinetic parameters k_{cat}, and K_{M}. The enzyme concentration was constrained to 0.05 nM as the concentration of the target served as the proxy for that of activated enzymes.

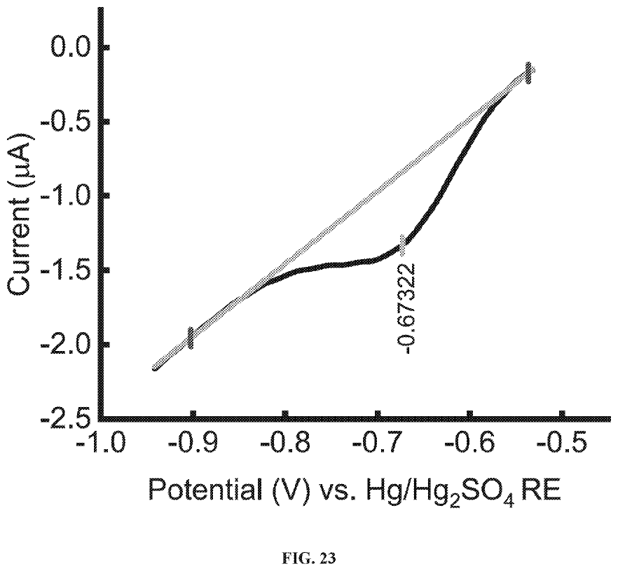

Gold Screen-printed Electrode Functionalization. The gold (Au)-SPE (C223BT, Metrohm) was first sonicated in acetone for 5 min to remove the impurity on the surface, followed by rinsing with deionized water for 30 s, dried by ultra-high pure nitrogen gas, and electrochemical cleaning by cycling voltammetry (CV) between −0.3 to 1.0 V in 0.1 M phosphate buffer (0.038 M NaH₂PO₄ and 0.061 M Na₂HPO₄, pH 7.4) versus a built-in Ag pseudo-reference electrode. The cleaned SPE was then rinsed with deionized water and dried by blowing ultra-high pure nitrogen gas. Thiol linked U₂₀ reporter with methylene blue (SEQ ID NO: 78, r7, Table 3) was reduced with 10 mM TCEP-HCl (Sigma Aldrich) for 10 min to break the disulfide bond before use. A total of 25 μL of 2 μM reduced U₂₀ reporter was cast on the WE for a 2 h incubation to form a close-packed self-assembled monolayer due to the thiol-gold interaction. The SPE was then rinsed with 10 mM Tris buffer (pH 8.0) and subsequently incubated in 2 mM 6-mercapto-1-hexanol (MCH, Sigma Aldrich, 451088) for 30 min. The alkanethiol layer serves as an insulator to reduce the background current while spacing U₂₀ molecules. Finally, the SPE was rinsed with 10 mM Tris buffer for 30 s to remove the physically adsorbed U₂₀. A final amount of approximately 50 pmol of reporters was attached to the surface of the WE (FIG. 23). The functionalized SPE was stored in 10 mM Tris buffer at 4° C. before use.

Endpoint Cas13a-electrochemical Measurement. The CRISPR-Cas13a master mix was prepared by adding the following components in tube with a top-down order, and two CRISPR reaction conditions were used (Table 5). The prepared CRISPR assay was incubated in the dark at room temperature for 10 min before adding to the WE for collateral cleavage.

TABLE 5
Preparation of CRISPR-Cas13a master mix in HEPES and
Tris buffers for Cas13-electrochemical measurement

| No. of steps | CRISPR reaction in HEPES buffer | CRISPR reaction in Tris buffer |
|---|---|---|
| (1) | Nuclease-free water | Nuclease-free water |
| (2) | 20 mM HEPES, pH 6.8 | 50 mM Tris-HCl, pH 8.0 |
| (3) | 9 mM MgCl₂ | 5 mM MgCl₂ |
| (4) | 45 nM protein | 45 nM protein |
| (5) | 2 U/μL RNase Inhibitor | 1 U/μL RNase Inhibitor |
| (6) | 22.5 nM crRNA | 22.5 nM crRNA |

Incubated for 10 min in the dark

| | | |
|---|---|---|
| (7) | various concentrations of target | various concentrations of target |
| Final Volume | 20 μL | 20 μL |

Electrochemical measurements were performed using an Autolab N Series Potentiostat. To measure the voltammetric responses, a volume of 40 μL of 10 mM Tris buffer (pH 8.0) containing 100 mM NaCl was dropped on the SPE to serve as the electrolyte. Square wave voltammetry (with a frequency of 20 Hz and 50 mV amplitude superimposed on a DC ramp from −0.5 V to 0 V versus the Ag reference electrode) was recorded from the U₂₀ reporter functionalized WE. The initial current was recorded before adding the target. Then the sensor was rinsed with nuclease-free water to remove the electrolyte and dried with nitrogen. The 20-μl reaction was added onto the WE and incubated at room temperature for 30 min, terminated by Proteinase K (25 μg/ml final; Thermo Scientific, EO0491) treatment. The sensor was rinsed, dried, and added with the electrolyte for SWV measurement of the final current. The SWV peak current before (ipcbefore) and after (ipcafter) the reaction was obtained from the built-in peak search function of the potentiostat software NOVA2.1. ΔI % was calculated from the SWV peak current by equation (3)

$$Δ I \% = \frac{( {ipc}_{before -} {ipc}_{after} )}{{ipc}_{before}} \times 100 \%$$

The theoretical LoD was determined by

$$LoD = 3 {S}_{blank} / m$$

where m is the slope of the best fitting line (sensor response to different target concentrations), and S_{blank} is the standard deviation of blank experiments.

Time-course Cas13a-electrochemical measurement. Time-course voltametric responses of Cas13 assays were obtained by SWV every 30 s over 2 min to measure the initial velocity of reaction, and over 20 min to record the full course. Each SWV measuring cycle included 5 s of deposition at a potential of −0.38 V to entirely reduce the MB for a maximal electron transfer, 5 s of the SWV measurement to obtain the peak current, followed by 20 s of waiting time. Before the reaction, 35 μl electrolyte was dropped on the WE and the initial peak current (ip₀) was obtained after 5-10 cycles of SWV scan for signal stabilization. A volume of 5 μl pre-assembled Cas13 reaction was then gently dispersed into the electrolyte with final concentrations of each component of reaction the same as described in Table 5 (Tris-buffered) with extra Tris (8.75 mM) and NaCl (87.5 mM) from the electrolyte. The peak current from each time point (ipₜ) was normalized to the corresponding initial peak current (ip₀) and plotted as a function of the time. To eliminate the background signal for curve fitting, the fold change of the normalized peak current of the target-containing reactions to that of the non-target controls (equation (5)) was calculated for each time point and then fitted with one-phase decay to determine the pseudo-first-order rate constant kobs (GraphPad)

$${( Background corrected peak current )}_{t} = \frac{{( {ip}_{t} / {ip}_{0} )}_{target}}{{( {ip}_{t} / {ip}_{0} )}_{NTC}}$$

where t is the SWV scan time (0, 30, 60. 90 s etc.).

Clinical sample RNA extraction and RT-qPCR. RNA for RT-qPCR detection was purified from 140 μl VTM leftover for the storage of clinical nasopharyngeal swab and eluted to 80 μl elution buffer using the QIAamp Viral RNA Mini Kit (Qiagen, 52904) per manufacturer's protocols. Purified RNAs were aliquoted and stored at −80° C. RT-qPCR quantification was performed according to Centers for Disease Control and Prevention instructions using 2019-nCOV RUO Kit (IDT, 10006713; N1-Forward: 5′-GACCCCAAAATCAGCGAAAT-3′ (SEQ ID NO: 143); N1-Reverse: 5′-TCTGGTTACTGCCAGTTGAATCTG-3′ (SEQ ID NO: 144); N1-Probe: FAM-ACCCCGCATTACGTTTGGTGGACC-BHQ1 (SEQ ID NO: 145)) and GoTaq Probe 1-Step RT-qPCR System (Promega, A6120) on a CFX96 Touch Real-Time PCR Detection System (BioRad) with the following cycling conditions: hold at 25° C. for 2 min, reverse transcription at 45° C. for 15 min, hold at 95° C. for 2 min, followed by 45 cycles with DNA denaturation at 95° C. for 3 s, and annealing and elongation at 55° C. for 30 s. Data were analyzed using CFX Maestro Software.

### Example 1—Tethering RBDs to the N- or C-terminus of LwaCas13a

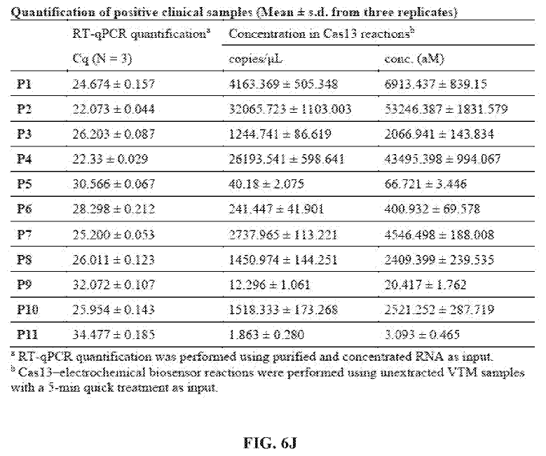

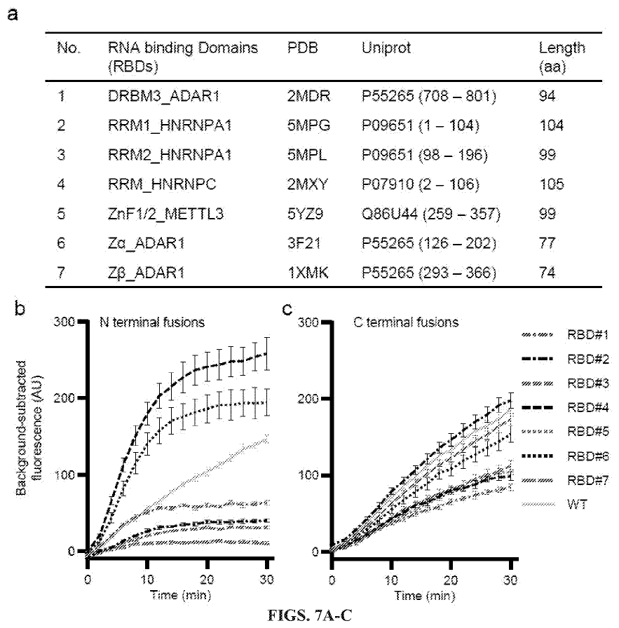

To enhance the collateral activity of LwaCas13a, the inventors first aimed to strengthen the interactions with its RNA substrates. It was hypothesized that fusing an RBD to Cas13a protein could facilitate the capture of adjacent target and reporter RNAs. Seven RBDs with different topologies (Lunde et al., 2007; Hentze et al., 2018; Bass, 2002; Beusch et al., 2017; Cieniková et al., 2014; Huang et al., 2019; Placido et al., 2007; Athanasiadis et al., 2005) were chosen as candidates, including the adenosine deaminases acting on RNA 1 (ADAR1) double-stranded RNA binding motif 3 (DRBM3, RBD #1) (Bass, 2002), heterogeneous nuclear ribonucleoprotein (hnRNP) A1 RNA recognition motif 1 (RRM1, RBD #2), RRM2 (RBD #3) (Beusch et al., 2017), hnRNP C RRM (RBD #4) (Cieniková et al., 2014), methyltransferase-like 3 (METTL3) zinc finger domain (ZnF, RBD #5) (Huang et al., 2019), ADAR1 Z-DNA/RNA binding domain α (Zα, RBD #6), and β (Zβ, RBD #7) (Placido et al., 2007; Athanasiadis et al., 2005) (FIGS. 1A and 7A).

RBDs were tethered either to the N- or C-terminus of LwaCas13a via a 32-residue XTEN linker to allow RBD's mobility (FIG. 1A). The resulting 14 RBD fusions were expressed and purified, and their collateral activities tested in the presence of a synthetic SARS-CoV-2 N gene fragment (FIGS. 1B, 1C, 7B, and 7C). 45 nM of WT LwaCas13a or an RBD fusion was complexed with 22.5 nM crRNA and 125 nM of RNaseAlert (IDT) was used as the fluorescent reporter. The endpoint fluorescence signal was calculated by subtracting background fluorescence (no-target control) from the on-target signal at 30 min. Comparison of signals produced by WT and RBD fusions showed that RBD #4N and RBD #6N marginally increased the endpoint fluorescent signal by 75.8% and 32.5%, respectively, while fluorescence signals produced by all other RBD fusions were similar to or less than WT LwaCas13a signal (FIGS. 1B, 1C, 7B, and 7C). RoseTTAFold (Baek et al., 2021) was then used to generate a LwaCas13a structure model (FIGS. 2A and 2B) based on the crystal structure of LbuCas13a (Liu et al., 2017) (PDB: 5XWP, 80% identity). This model indicated that the N-terminus of LwaCas13a could be buried within the crRNA binding cleft, while the C-terminus might be very close to the catalytic center of the protein. Linking RBDs to the N- or C-terminus may disrupt Cas13a folding or active-site configuration, providing a possible explanation why 9 of the 14 RBD fusions led to the decreased activity (FIGS. 1B and 1C).

### Example 2—Identifying Active Site-Proximal Loops Within LwaCas13a for Inserting RBDs

By carefully examining the LwaCas13a structural model, a unique β-hairpin loop G410-G425 (Loop 1) was identified within the HEPN1 domain that is located above the active site cleft (FIGS. 2B and 2C). As such, Loop 1 was considered to be an optimal insertion site for the proper orientation of RBDs and enhanced RNA binding. Thus, the seven selected RBDs were fused between residues N415 and N416 at the tip of Loop 1 (FIG. 2C) without linkers (due to the flexibility of Loop 1), and the collateral activity of the purified fusion proteins was tested using the same conditions as in FIGS. 1B and 1C. Five of the Loop 1 RBD fusions (RBD #IL, #2L, #3L, #4L and #6L) had 59%, 216%, 468%, 518% and 174% higher endpoint fluorescence signals than WT after 30 minutes exposure to the synthetic SARS-CoV-2 N gene RNA target (FIG. 2D). Similar collateral activity improvement was also observed with two other synthetic RNA targets (FIGS. 8A-C), where RBD #2L, #3L, #4L and #6L revealed 164%, 264%, 241%, and 171% increased activity relative to WT with a synthetic SARS-CoV-2 RdRp gene target and 59%, 102%, 91%, and 38% enhanced activity with a ssRNA1 target (Gootenberg et al., 2017) (FIGS. 8A-C). The 99 aa hnRNP A1 RRM2 (FIG. 2E) and 105 aa hnRNP C RRM (FIG. 2F) Loop 1 fusions (RBD #3L and #4L) showed the highest collateral activities for all three tested synthetic RNA targets.

It was reasoned that RBD insertion position might affect its orientation and RNA substrate capture, so its insertions were varied between amino acids N415, N416, and K417 near the tip of Loop 1 and beyond its β-sheet secondary structure (FIG. 2C). All RBD #3 and #4 Loop 1 fusions demonstrated significantly increased collateral activity, and insertion between amino acid residue N415 and N416 (RBD #3L and #4L) exhibited comparable or higher collateral activities among all tested insertion sites (FIG. 2G and FIGS. 8D-F). Additionally, inserting tandem RBDs, that is, RBD #3-linker-RBD #4 or RBD #4-linker-RBD #3 into the same position did not further improve collateral activity (FIGS. 8G-H). In contrast, RBD #3 and #4 insertions at another active site-proximal loop within the HEPN2 domain of LwaCas13a (Loop 2, N992-G1004, FIGS. 2A-C) led to a complete loss of collateral activity, even after the addition of flexible GSSG (SEQ ID NO: 123) or 3×GGGGS (SEQ ID NO: 124) linkers (FIG. 2G). Collectively, inserting RBDs to the optimal site in Loop 1 resulted in two LwaCas13a variants, RBD #3L and #4L, with the highest collateral activity improvement.

### Example 3—Reporter Length and Context Requirements for Optimal Collateral Cleavage

Further analyzing the structure model, the distance between the tip of Loop 1 (N415) and the LwaCas13a catalytic residues was estimated to be approximately 35 Å. Although this distance might vary due to the flexibility of Loop 1, an RNA reporter with a length over 10-nt would allow it to efficiently span the distance between the RBD binding site and the HEPN active site. Additionally, the hnRNP A1 RRM2 (RBD #3, FIG. 2E) binds at least four nucleotides with a minimal sequence preference (Beusch et al., 2017), while the hnRNP C RRM (RBD #4, FIG. 2F) preferably binds tracts of four uridines (Cieniková et al., 2014). Thus, the 7-nt RNaseAlert substrate used in the above assays may be suboptimal due to its insufficient length. Moreover, LwaCas13a has a U-U preference for collateral cleavage (Gootenberg et al., 2018) and replacement of the RNaseAlert with a polyU-linked fluorophore-quencher pair improves WT collateral activity (Arizti-Sanz et al., 2020).

Next, the collateral activity of target-bound RBD #3L and WT was

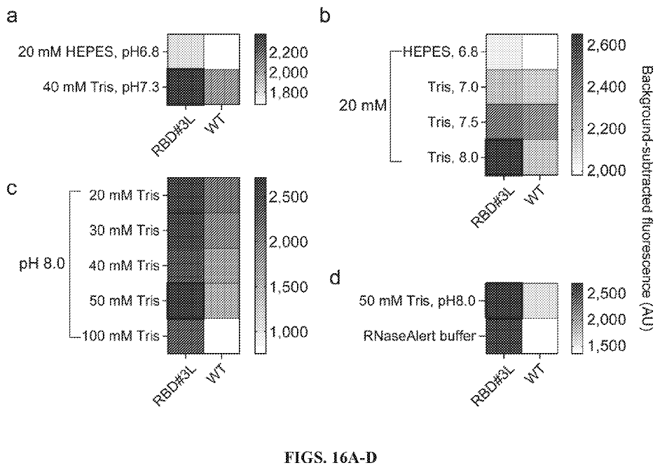

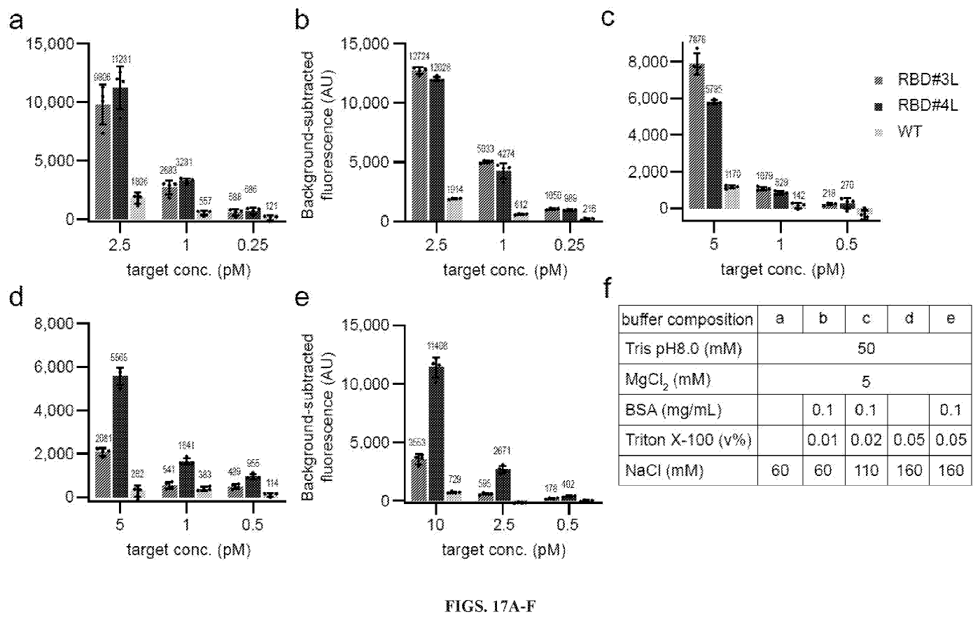

tested with 5′-6-Carboxyfluorescein (6-FAM)-labeled-polyU reporters of four different lengths: 5-nt, 11-nt, 15-nt, and 20-nt. Reactions were initiated by incubating with the synthetic SARS-CoV-2 N gene fragment. Aliquots were collected at 0, 5, 10, 15, and 30 min and resolved on a denaturing gel to visualize the time-course of collateral cleavage activity (FIGS. 9A-E). Both RBD #3L, RBD #4L, and the WT all showed increased reporter cleavage in a reporter length-dependent manner. Semi-quantitative analysis of the cleaved products exhibited more efficient collateral cleavage by RBD #3L and RBD #4L than WT in reactions using U₁₁, U₁₅ or U₂₀, but not U₅ reporters (FIGS. 3A and 3B and 9A-E). The cleavage of RBD #3L, #4L, and WT was further quantified using the self-quenched U₅, RNaseAlert (N₇) and U₁₁ reporters (FIGS. 3C and 3D). The overall fluorescence intensity and initial collateral cleavage rates of all three proteins were the lowest when RNaseAlert (N₇) was used, demonstrating the U-U preference of LwaCas13a. By using the Un reporter, WT, RBD #3L and #4L initial collateral cleavage rates were 1.5-, 4.7-, and 5.8-fold higher than those of the U₅ reporter, showing a reporter length requirement for the optimal collateral activity of RBD fusions higher than that of the WT (FIGS. 3C-D). By using the optimized reaction buffer conditions (50 mM Tris pH 8.0 and 5 mM Mg²⁺), assays employing RBD #3L or #4L with U₁₁ reporter increased the collateral cleavage rate up to 58-fold relative to WT LwaCas13a with RNaseAlert reporter (FIGS. 3C, 3D, and 11). Further optimizing reaction buffer compositions (FIGS. 16-17) improved the analytical limit-of-detection (LoD) of the WT to ˜0.3 pM (final concentration in the reaction), while RBD #3L and RBD #4L reached the LoDs of 9 and 13 fM, respectively (FIGS. 10A-F and 18A-F). Collectively, combining with reporters with extended length (≥11 nucleotides), RBD #3L and #4L exhibited a constantly enhanced collateral activity relative to the WT under all tested conditions.

### Example 4—Characterization of RBD #3L and RBD #4L

To systematically evaluate the collateral activity of RBD #3L and #4L, an unbiased test was performed using 11 crRNAs tiled along the length of a ssRNA1 target (Gootenberg et al., 2018) (FIGS. 4A-B and 19A). RBD #3L and #4L demonstrated enhanced collateral activity at all sites over a course of 120 min, regardless of the spacer sequence (FIG. 4A). Fluorescence signal produced by two RBD fusions at an early-point (30 min) ranged from a 2.6- to 13.4-fold higher than WT signal, with an average of 5.3-fold increase (FIG. 4B), indicating an accelerated reaction speed with all tested spacers. These results demonstrate that RBD #3L and #4L have enhanced collateral activity while retaining the programmability.

To test if the target recognition specificity would be affected by the enhanced collateral activity of RBD #3L and #4L, single mutations (MMs) were introduced at every two nucleotides, or consecutive double mutations (DMs) at every four nucleotides across a 28-nt synthetic SARS-CoV-2 N gene target (FIG. 19B). Comparison of the relative fold-change in background-subtracted fluorescence produced upon incubation with each mismatched target versus that of the perfect-match (PM) target revealed no significant difference in the normalized fluorescence signal, indicating that RBD #3L and #4L maintain the specificity of WT (FIGS. 4C and 19B). Collectively, these data indicated that RBD #3L and #4L exhibit enhanced collateral activity in a spacer-independent manner without compromising the intrinsic programmability and targeting specificity of LwaCas13a, suggesting that these two fusion proteins could be widely used for RNA detection.

To further characterize RBD #3L and #4L, the inventors measured their RNA binding affinity (K_{D}) and catalytic efficiency (k_{cat}/K_{M}) in comparison with the WT. Electrophoretic mobility shift assays showed K_{D} values of 23, 18, and 46 nM using a 36-nucleotide target RNA binding with the complex of the catalytically inactive HEPN2 domain mutants (R1046A/H1051A), that is, dRBD #3L, dRBD #4L and dWT, and the crRNA (FIGS. 11A-B). By using a longer target (105 nucleotides), K_{D} values were measured as 12, 11, and 16 nM for dRBD #3L, dRBD #4L, and dWT RNPs, respectively (FIGS. 11C-D), again showing a higher target RNA binding affinity of the RBD fusions than the WT. In addition to the enhanced target binding, the reporter binding affinity was also improved by RBD fusions. The estimated K_{D} values were ˜0.23, 0.15, and 0.47 μM for U₂₀ reporter binding with dRBD #3L, dRBD #4L, and dWT, respectively (FIGS. 11E-G). In agreement with the K_{D} of reporter-protein binding, the Michaelis-Menten collateral cleavage kinetics using U₁₁ as the substrate showing K_{M} values were 1.9, 0.48, and 7 μM and the catalytic efficiency (k_{cat}/K_{M}) of reporter cleavage was 1.7×10₇, 1.5×10₇, and 3.9×10₆ M₋₁s₋₁ for RBD #3L, RBD #4L, and the WT, respectively (FIGS. 12A-E). The catalytic efficiency of RBD #3L and RBD #4L remained two-to threefold higher than that of the WT in reactions using reaction buffer supplemented with 0.01% bovine serum albumin and Triton X-100 (FIGS. 20A-E). Taken together, RBD #3L and #4L exhibited increased binding affinities to both target and reporter RNAs, as well as improved collateral cleavage efficiency.

### Example 5—Detect Various Targets using Cas13a Variants

To evaluate the broader application of these LwaCas13a variants, RBD #3L and #4L were both used to detect a variety of clinically relevant RNA targets, including RNA targets from SARS-CoV-2, Zika, Dengue, and Ebola viruses, and two short microRNA (miR) targets, miR-19b as a predictive biomarker for cardiovascular death (Karakas et al., 2017) and miR-2392 as a diagnostic marker for COVID-19 severity

(McDonald et al., 2021). The matrices and contaminants from biofluids usually reduce the sensitivity of a CRISPR-based detection (Gootenberg et al., 2017; Shinoda et al., 2021; Arizti-Sanz et al., 2020). To mimic a direct detection of RNA targets from the complex composition of their diagnostic samples (Santiago et al., 2013; Broadhurst et al., 2016; Byrne et al., 2020; Reijns et al., 2020), these synthetic RNA targets were spiked into the corresponding biological specimen types that contain background RNAs (bgRNA). Prior to the fluorescence plate reader assay, biofluid samples were treated with the HUDSON methods (Arizti-Sanz et al., 2020; Myhrvold et al., 2018; Barnes et al., 2020; Shan et al., 2019) to simulate the release of RNA from viral particles or exosomes, and eliminate the nuclease activity from the samples.

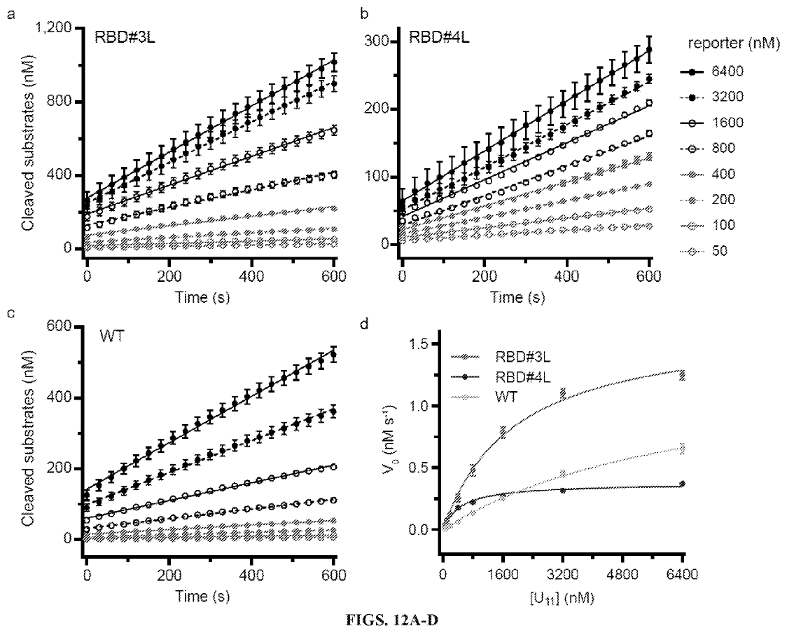

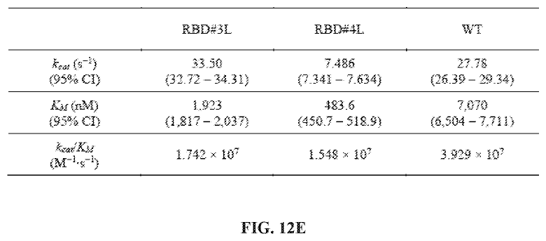

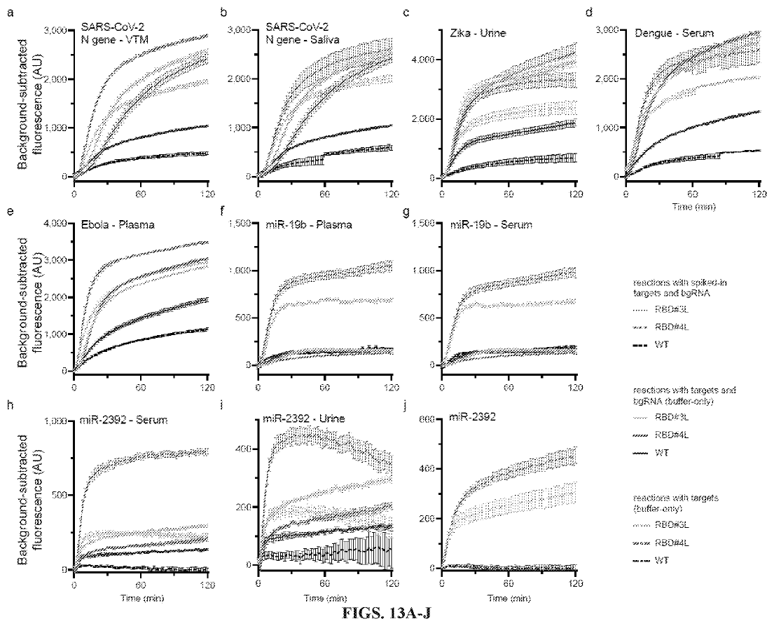

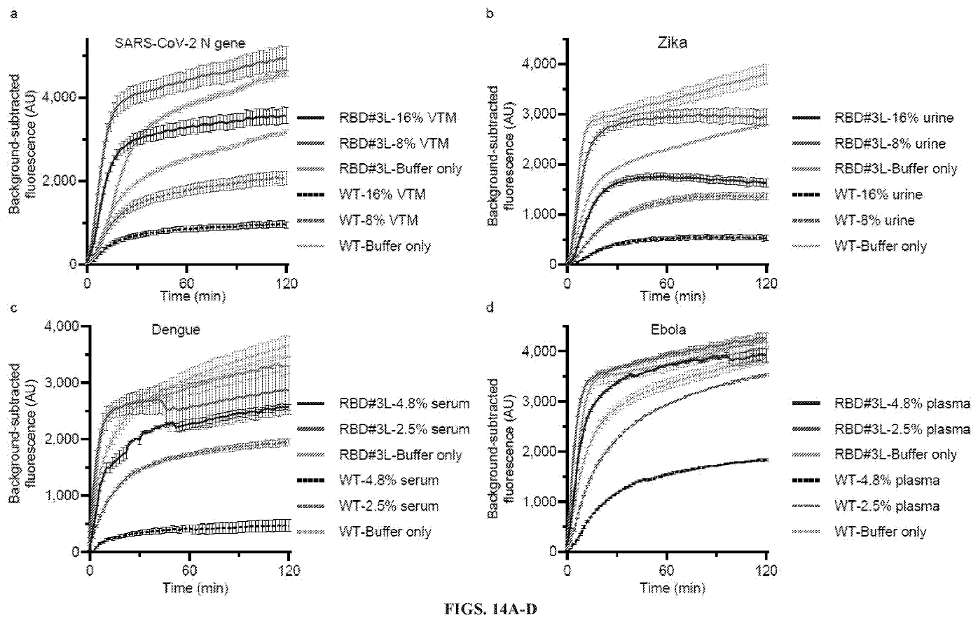

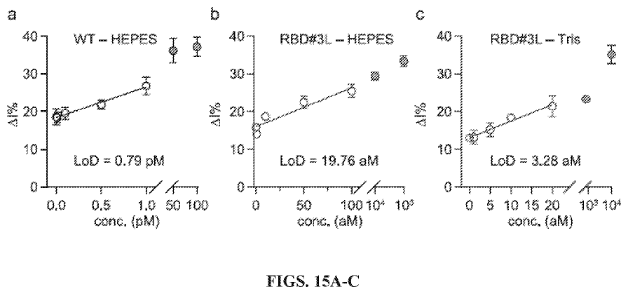

In detecting the SARS-CoV-2 N gene target, with the addition of 8% viral transportation medium (VTM) or 8% saliva to the reaction buffer-alone, WT showed 40% decreased activity, while RBD #3L demonstrated comparable or slightly increased activity and RBD #4L exhibited 2.6- or 2.2-fold increased activity (FIGS. 5A, 13A, and 13B). Detecting the same target in reactions containing 16% VTM, RBD #3L retained 78% of the signal, however, only 30% activity remained in WT, compared to buffer-alone reactions (FIG. 14A). Using similar approaches, Zika, Dengue, and Ebola virus RNA targets were detected in reactions containing 8% urine, 2.5% serum, or 2.5% plasma, respectively. WT showed as much as 68% reduced signal in the presence of those complex matrices (FIGS. 5B-5D and 13C-13E), while RBD #3L and #4L remained good activity and showed 2.5- to 6.7-fold signals relative to WT. When higher concentration of these matrices was used (i.e., 16% urine, 4.8% serum and 4.8% plasma), RBD #3L still retained 43%, 78%, and 93% of the signal compared to those reactions in buffer only conditions (FIGS. 14B-14D). Likewise, RBD #3L and RBD #4L collateral activity in response to a 23-nt miR-19b target was 4.7- and 7.3-fold relative to WT in reactions containing 2.5% serum or 2.5% plasma (FIGS. 5E and 13F-13H). Finally, RBD #4L showed 28- and 14-fold activities relative to WT in detecting a 20-nucleotide miR-2392 (discovered only in serum and urine (McDonald et al., 2021)) from samples containing 2.5% serum and 8% urine, respectively (FIGS. 5f and 13H-J). Collectively, RBD #3L and #4L showed enhanced collateral cleavage over the WT in various buffer conditions (FIGS. 5 and 13 and 15-17). In complex reaction conditions, such as the biofluid-containing samples, RBD #4L maintained more robust collateral activity than RBD #3L (FIGS. 5 and 13 and 14), suggesting that the intrinsic RBD #4-PolyU binding affinity23 facilitates effective collateral cleavage (FIG. 21). RBD #3L and #4L exhibited superior robustness in detecting target RNAs from biofluid-containing samples, holding great potential for field-deployable detection of various RNAs for diagnostic purposes.

### Example 6—Ultrasensitive and Amplification-Free RNA Detection

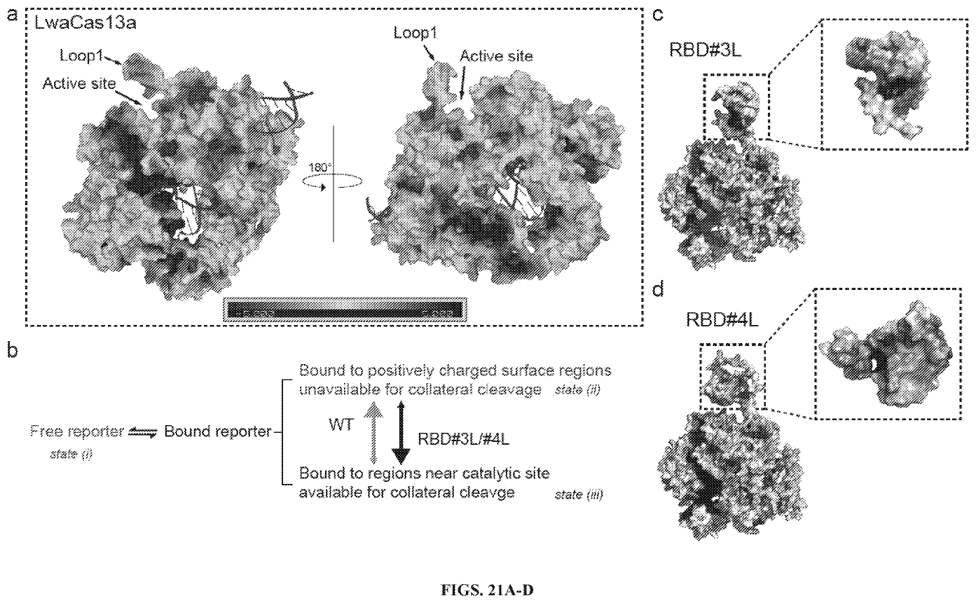

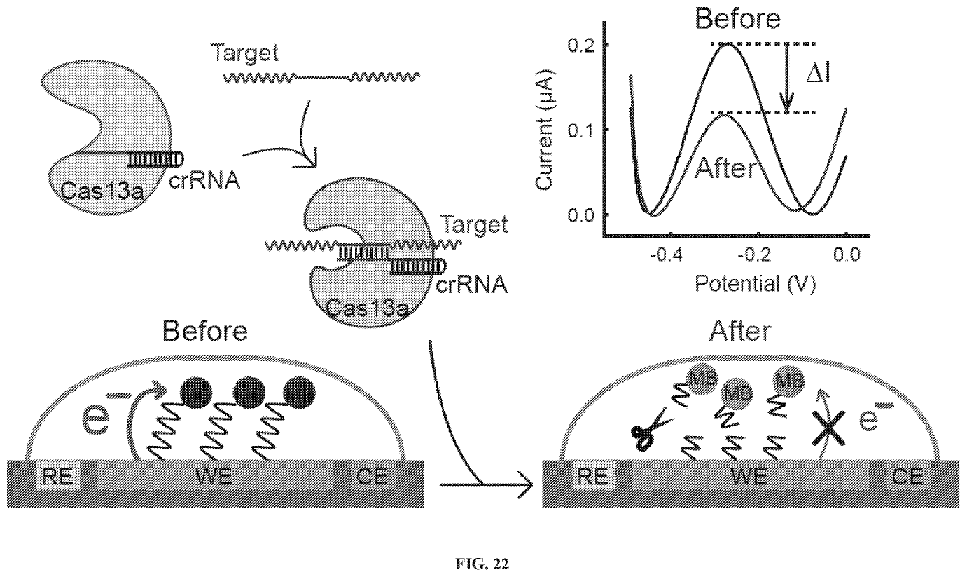

To demonstrate that RBD #3L and #4L could be employed for on-site diagnosis of viral infection, their target-activated collateral cleavage reaction was integrated into a screen-printed electrochemical (SPE) device (FIG. 22). The working electrode (WE) of this device was first functionalized with the U₂₀ reporter tagged with methylene blue (MB), a redox-active electrochemical reporter, the surface density of which was estimated to be ˜25 pmol/mm² (FIG. 23). The electron transfer between the WE and redox-active MB was acquired by square wave voltammetry (SWV) (Dai et al., 2019) (FIG. 22). Cleavage of MB-tagged U₂₀ reporters from the WE by Cas13a collateral activity is triggered by recognition of an RNA target and results in a decrease in the peak electrochemical current expressed by ΔI % (FIG. 22).

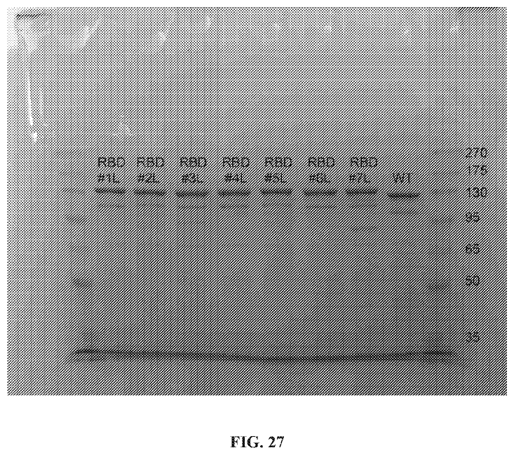

Evaluation of the analytical performance of this Cas13a—electrochemical system using serial dilutions of a synthetic SARS-CoV-2 N gene target (1aM to 100 pM) revealed an estimated limit of detection (LoD) of 500 fM for WT LwaCas13a (FIGS. 6A and 15A and 27). By using RBD #3L, the LoD of this system was dramatically lowered to 10 aM (FIGS. 6B-C and 15B-C and 27), demonstrating 50,000-fold more sensitivity than WT. Further measurement of the time-course electrochemical signals every 30 s by repeated SWV scans (Methods; FIG. 23) and demonstrated clear discrimination of the reactions containing 10 aM targets from the non-target control by using RBD #3L and #4L, while the WT showed only background-level signal (FIGS. 24A-F). The best-fit pseudo-first-order rate constants (that is, observed rate constant, kobs) at a target concentration of 10 aM were 0.007356 s−1, 0.003639 s−1, and 1.213×10-8 s−1 for RBD #3L, #4L, and the WT, respectively, showing over 105-fold improvement of the kobs upon RBD fusion.

To test the efficacy of this system for genomic RNA detection, heat-inactivated SARS-CoV-2 samples from infected Vero E6 cells (ATCC VR-1986HK) were treated with a 5 min extraction-free procedure (Ladha et al., 2020), serially diluted, and analyzed. No difference was detected from the electrochemical signal produced by the blank (no target) and the 10,000 aM virus RNA sample in a WT-based system (FIG. 6D). However, an RBD #3L-based system detected a significant difference (p=0.0171, unpaired t-test with Welch's correction) between the blank and 1 aM (final concentration in the reaction) viral RNA sample (FIG. 6E), consistent with the performance of RBD #3L in detecting the synthetic viral RNA target. Similar results were observed when the RBD #4L was used in place of RBD #3L in this system, with a significant difference observed between the blank and 1 aM target reactions (FIG. 6F). The ten-fold further reduction of LoD in detecting genomic RNA compared to the synthetic target was also observed by a recent study (Nalefski et al., 2021). Finally, the RBD #3L system was used to test VTM leftovers for storage of the clinical SARS-CoV-2 nasopharyngeal swab biospecimens. These VTM samples were all heat-inactivated and labeled as SARS-CoV-2 positive or negative based on the PCR or isothermal nucleic acid amplification tests used at UConn Health for diagnostic purposes. The RBD #3L-electrochemical system successfully discriminated all three positive samples from the three negative samples (FIG. 6G). As such, RBD-LwaCas13a proteins engineered with improved collateral activity can be integrated into diverse detection platforms to detect pathogen-or host-derived RNA targets in clinical laboratory or point-of-care settings.

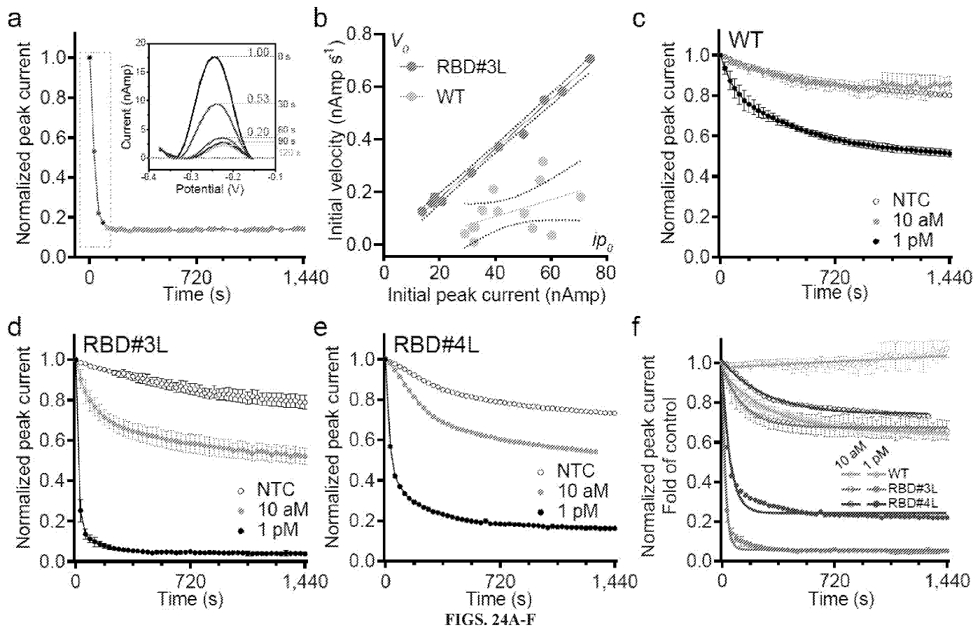

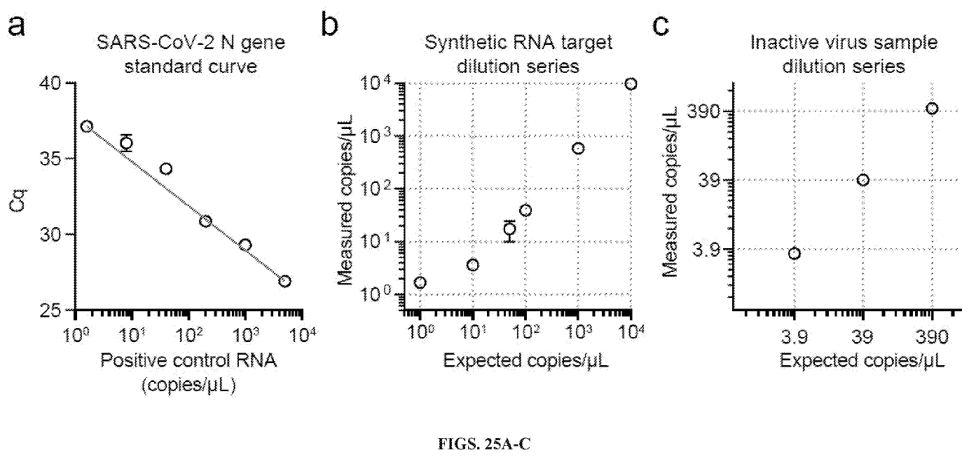

Finally, the performance of the WT, RBD #3L, and RBD #4L systems in detecting clinical SARS-CoV-2 samples were compared (FIGS. 6G-I). The VTM leftovers for storage of the nasopharyngeal swab biospecimens were all heat-inactivated and labeled as SARS-CoV-2 positive or negative on the basis of the PCR or isothermal nucleic acid amplification tests used at UConn Health for diagnostic purposes. The specific viral loads were first quantified by performing quantitative PCR with reverse transcription (RT-qPCR) using purified RNA from these clinical samples (FIGS. 25A-C). Eleven samples were confirmed positive with C_{q} values ranging from 34 to 22, equivalent to a viral load from 1.9 to 32,066 copies per microliter (3 aM to 53 fM) in electrochemical assay (FIG. 6J) and ten samples were determined to be negative. The raw samples were subjected to 5-min quick treatment at 95° C. to eliminate nuclease activity and facilitate viral RNA release and tested using an electrochemical sensor coupled with the WT, RBD #3L, and RBD #4L, respectively (FIGS. 6G-I). A detection threshold was set to two standard deviations above the mean of blank samples 17 (non-target control reactions with empty VTM in place of clinical samples), allowing distribution of more than ˜95% of the clinical negative samples below the detection criteria. Following this standard, RBD #3L detected nine and RBD #4L detected ten positive samples within 30 min (FIGS. 6H-J). One positive sample (P11) with a Cq value of 34 (1.9copies per microliter in the electrochemical assay) could not be detected by both RBD #3L and #4L, while P9 with a Cq value of 32 (12 copies per microliter) could be successfully detected by RBD #4L. When ten negative samples were tested by using either RBD #3L or #4L, no false-positive data was shown in our analysis, showing the high accuracy of our method. Notably, the WT LwaCas13a cannot detect the positive samples with the highest available concentrations (P2, P4, and P8 with Cq values of 22, 22, and 26, respectively; FIG. 6G and 6J). The remarkably improved sensitivity of RBD #3L and #4L compared to WT on the electrochemical biosensor may be attributed to a synergistic effect of several factors, including the enhanced reporter RNA binding with the RBD proximal to the active site of LwaCas13a and the surface chemistry at the reaction interface. On the basis of these findings, RBD-LwaCas13a proteins engineered with improved collateral activity can be integrated into diverse detection platforms to detect pathogen- or host-derived RNA targets in the clinical laboratory or point-of-care settings.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abudayyeh et al., RNA targeting with CRISPR-Cas13. Nature 550, 280-284 (2017).
Abudayyeh et al., A cytosine deaminase for programmable single-base RNA editing. Science 365, 382-386 (2019).
Ackerman et al., Massively multiplexed nucleic acid detection with Cas13. Nature 582, 277-282 (2020).
Arizti-Sanz et al., Streamlined inactivation, amplification, and Cas13-based detection of SARS-CoV-2. Nature Communications 11, 5921 (2020).
Arnaout et al., The Limit of Detection Matters: The Case for Benchmarking Severe Acute Respiratory Syndrome Coronavirus 2 Testing. Clinical Infectious Diseases (2021).
Athanasiadis et al., The crystal structure of the Zbeta domain of the RNA-editing enzyme ADAR1 reveals distinct conserved surfaces among Z-domains. Journal of Molecular Biology 351, 496-507 (2005).
Baek et al., Accurate prediction of protein structures and interactions using a three-track neural network. Science 373, 871-876 (2021).
Barnes et al., Deployable CRISPR-Cas13a diagnostic tools to detect and report Ebola and Lassa virus cases in real-time. Nature Communications 11, 4131 (2020).
Bass, RNA editing by adenosine deaminases that act on RNA. Annual Review of Biochemistry 71, 817-846 (2002).
Beusch et al., Tandem hnRNP A1 RNA recognition motifs act in concert to repress the splicing of survival motor neuron exon 7. Elife 6 (2017).
Broadhurst et al., Diagnosis of Ebola Virus Disease: Past, Present, and Future. Clinical Microbiology Reviews 29, 773-793 (2016).
Broughton et al., CRISPR-Cas12-based detection of SARS-CoV-2. Nature Biotechnology 38, 870-874 (2020).
Bruch et al., CRISPR/Cas13a-powered electrochemical microfluidic biosensor for nucleic acid amplification-free miRNA diagnostics. Advanced Materials 31, e1905311 (2019).
Bruch et al., CRISPR-powered electrochemical microfluidic multiplexed biosensor for target amplification-free miRNA diagnostics. Biosensors and Bioelectronics 177, 112887 (2021).
Byrne et al., Saliva alternative to upper respiratory swabs for SARS-CoV-2 diagnosis. Emerging Infectious Diseases 26, 2770-2771 (2020).
Charles et al., Engineering improved Cas13 effectors for targeted post-transcriptional regulation of gene expression. bioRxiv, 2021.2005.2026.445687 (2021).
Chu et al., Rationally designed base editors for precise editing of the sickle cell disease mutation. CRISPR J. 4, 169-177 (2021)
Cieniková et al., Structural and mechanistic insights into poly(uridine) tract recognition by the hnRNP C RNA recognition motif. Journal of the American Chemical Society 136, 14536-14544 (2014).
Cox et al., RNA editing with CRISPR-Cas13. Science 358, 1019-1027 (2017).
Dai et al., Exploring the Trans-Cleavage Activity of CRISPR-Cas12a (cpf1) for the Development of a Universal Electrochemical Biosensor. Angew Chem Int Ed Engl 58, 17399-17405 (2019).
Dominguez et al., Sequence, structure, and context preferences of human RNA binding proteins. Molecular Cell 70, 854-867 e859 (2018).
East-Seletsky et al., Two distinct RNase activities of CRISPR-Cas13a enable guide-RNA processing and RNA detection. Nature 538 (7624), 270-273 (2016).
East-Seletsky et al., RNA targeting by functionally orthogonal type VI-A CRISPR-Cas enzymes. Molecular Cell 66 (3), 373-383.e3 (2017).
Fozouni et al., Amplification-free detection of SARS-CoV-2 with CRISPR-Cas13a and mobile phone microscopy. Cell 184, 323-333 e329 (2021).
Gootenberg et al., Nucleic acid detection with CRISPR-Cas13a/Cas13a. Science 356, 438-442 (2017).
Gootenberg et al., Multiplexed and portable nucleic acid detection platform with Cas13, Cas12a, and Csm6. Science 360, 439-444 (2018).
Guo et al., SARS-CoV-2 detection with CRISPR diagnostics. Cell Discov 6, 34 (2020).
Hajian et al., Detection of unamplified target genes via CRISPR-Cas9 immobilized on a graphene field-effect transistor. Nature Biomedical Engineering 3, 427-437 (2019).
Han et al., RNA-protein interaction mapping via MS2- or Cas13-based APEX targeting. Proc. Natl Acad. Sci. USA 117, 22068-22079 (2020).
Hardinge & Murray, Reduced false positives and improved reporting of loop-mediated isothermal amplification using quenched fluorescent primers. Sci. Rep. 9, 7400 (2019).
Hentze et al., A brave new world of RNA-binding proteins. Nature Reviews Molecular Cell Biology 19, 327-341 (2018).
Huang et al., Solution structure of the RNA recognition domain of METTL3-METTL14 N6-methyladenosine methyltransferase. Protein & Cell 10, 272-284 (2019).
Huang et al., Ultra-sensitive and high-throughput CRISPR-powered COVID-19 diagnosis. Biosens Bioelectron 2020, 164, 112316.
Jolma et al., Binding specificities of human RNA-binding proteins toward structured and linear RNA sequences. Genome Research 30, 962-973 (2020).
Joung et al., Detection of SARS-CoV-2 with SHERLOCK one-pot testing. The New England Journal of Medicine 383, 1492-1494 (2020).
Jumper, J. et al. Highly accurate protein structure prediction with AlphaFold. Nature 596, 583-589 (2021).
Kaminski et al., CRISPR-based diagnostics. Nature Biomedical Engineering 5, 643-656 (2021).
Karakas et al., Circulating microRNAs strongly predict cardiovascular death in patients with coronary artery disease—results from the large AtheroGene study. European Heart Journal 38, 516-523 (2017).
Kellner et al., SHERLOCK: nucleic acid detection with CRISPR nucleases. Nat Protoc 14, 2986-3012 (2019).
Khan et al., Isothermal SARS-CoV-2 diagnostics: tools for enabling distributed pandemic testing as a means of supporting safe reopenings. ACS Synth. Biol. 9, 2861-2880 (2020).
Konermann et al., Transcriptome Engineering with RNA-Targeting Type VI-D CRISPR Effectors. Cell 173, 665-676 e614 (2018).
Ladha et al., A 5-min RNA preparation method for COVID-19 detection with RT-qPCR. Preprint at medRxiv https://doi.org/10.1101/2020.05.07.20055947 (2020).
Lee et al., Ultrasensitive CRISPR-based diagnostic for field-applicable detection of Plasmodium species in symptomatic and asymptomatic malaria. Proceedings of the National Academy of Sciences 117, 25722 (2020).
Li et al., A chemical-enhanced system for CRISPR-Based nucleic acid detection. Biosensors and Bioelectronics 192, 113493 (2021).
Liu et al., The molecular architecture for RNA-guided RNA cleavage by Cas13a. Cell 170, 714-726 e710 (2017).
Liu et al., Accelerated RNA detection using tandem CRISPR nucleases. Nature Chemical Biology (2021).
Lunde et al., RNA-binding proteins: modular design for efficient function. Nature Reviews Molecular Cell Biology 8, 479-490 (2007).
Mammoth Biosciences, Inc. SARS-CoV-2 DETECTR™ Reagent Kit. 2020, available on the world wide web at fda.gov/media/141765/download.
McDonald et al., Role of miR-2392 in driving SARS-CoV-2 infection. Cell Rep. 37, 109839 (2021).
McDonald et al., The Great Deceiver: miR-2392's Hidden Role in Driving SARS-CoV-2Infection. bioRxiv, 2021.2004.2023.441024 (2021).
Myhrvold et al., Field-deployable viral diagnostics using CRISPR-Cas13. Science 360, 444-448 (2018).
Nalefski et al., Kinetic analysis of Cas12a and Cas13a RNA-Guided nucleases for development of improved CRISPR-Based diagnostics. iScience 24, 102996 (2021).
Nano, A., Furst, A.L., Hill, M.G. & Barton, J.K. DNA Electrochemistry: Charge-Transport Pathways through DNA Films on Gold. Journal of the American Chemical Society 143, 11631-11640 (2021).
Nguyen et al., Enhancement of trans-cleavage activity of Cas12a with engineered crRNA enables amplified nucleic acid detection. Nature Communications 11, 4906 (2020).
Ning et al., A smartphone-read ultrasensitive and quantitative saliva test for COVID-19. Sci Adv 7 (2021).
Oakes et al., Profiling of engineering hotspots identifies an allosteric CRISPR-Cas9 switch. Nat. Biotechnol. 34, 646-651 (2016).
Ooi et al., An engineered CRISPR-Cas12a variant and DNA-RNA hybrid guides enable robust and rapid COVID-19 testing. Nature Communications 12, 1739 (2021).
Park et al., Extension of the crRNA enhances Cpf1 gene editing in vitro and in vivo. Nature Communications 2018, 9 (1), 3313.
Patchsung et al., Clinical validation of a Cas13-based assay for the detection of SARS-CoV-2 RNA. Nature Biomedical Engineering 4, 1140-1149 (2020).
Placido et al., A left-handed RNA double helix bound by the Z alpha domain of the RNA-editing enzyme ADAR1. Structure 15, 395-404 (2007).
Qin et al., Rapid and fully microfluidic Ebola virus detection with CRISPR-Cas13a. Acs Sensors 4, 1048-1054 (2019).
Reijns et al., A sensitive and affordable multiplex RT-qPCR assay for SARS-CoV-2 detection. PLOS Biology 18, e3001030 (2020).
Richter et al., Phage-assisted evolution of an adenine base editor with improved Cas domain compatibility and activity. Nat. Biotechnol. 38, 883-891 (2020).
Santiago et al., Analytical and clinical performance of the CDC real time RT-PCR assay for detection and typing of dengue virus. PLOS Neglected Tropical Diseases 7, e2311 (2013).
Shan et al., High-Fidelity and Rapid Quantification of miRNA Combining crRNA Programmability and CRISPR/Cas13a trans-Cleavage Activity. Anal Chem 91, 5278-5285 (2019).
Sherlock Biosciences, Inc. Sherlock™ CRISPR SARS-CoV-2 Kit. 2020, available on the world wide web at fda.gov/media/137746/download.
Shinoda et al., Amplification-free RNA detection with CRISPR-Cas13. Communications Biology 4, 476 (2021).
Shmakov et al., Discovery and Functional Characterization of Diverse Class 2 CRISPR-Cas Systems. Mol Cell 60, 385-397 (2015).
Son et al., Sensitive and multiplexed RNA detection with Cas13 droplets and kinetic barcoding. medRxiv, 2021.2008.2002.21261509 (2021).
Zhang et al., Structural basis for the RNA-guided ribonuclease activity of CRISPR-Cas13d. Cell 175, 212-223 e217 (2018).
Zhang et al., A protocol for detection of COVID-19 using CRISPR diagnostics. 2020, available on the world wide web at broadinstitute.org/files/publications/special/COVID-19%20detection%20(updated).pdf.
Zou et al., Evaluation and improvement of isothermal amplification methods for point-of-need plant disease diagnostics. PLOS ONE 15, e0235216 (2020).

1. An engineered Cas13 enzyme comprising a binding domain fusion, wherein the engineered Cas13 enzyme has enhanced collateral activity as compared to a Cas13 enzyme.

2-3. (canceled)

4. The engineered enzyme of claim 1, wherein the binding domain is an RNA-binding domain, a DNA-binding domain, or an aptamer.

5. (canceled)

6. The engineered protein of claim 1, wherein the binding domain is an RNA-binding domain, wherein the RNA-binding domain is an RNA recognition motif, a double-stranded RNA binding domain, or a zinc-finger RNA binding domain.

7. (canceled)

8. The engineered protein of claim 6, wherein the RNA-binding domain has a sequence selected from the group consisting of: ADAR1⁷⁰⁸⁻⁸⁰¹ (amino acids 416-509 of SEQ ID NO: 2), hnRNPA1¹⁻¹⁰⁴ (amino acids 416-519 of SEQ ID NO: 3), hnRNPA1⁹⁸⁻¹⁹⁶ (amino acids 416-514 of SEQ ID NO: 4), hnRNPC²⁻¹⁰⁶ (amino acids 416-520 of SEQ ID NO: 5), METTL3²⁵⁹⁻³⁵⁷ (amino acids 416-514 of SEQ ID NO: 6), ADAR1¹²⁶⁻²⁰² (amino acids 416-492 of SEQ ID NO: 7), and ADAR1²⁹³⁻³⁶⁶ (amino acids 416-489 of SEQ ID NO: 8).

9. The engineered protein of claim 6, wherein the RNA-binding domain is the heterogeneous nuclear ribonucleoprotein (hnRNP) A1 RNA recognition motif 1 (RRM1) or the hnRNP C RNA recognition motif (RRM).

10. The engineered protein of claim 1, wherein the binding domain is fused to the N-terminus or the C-terminus of the Cas13 enzyme.

11. The engineered protein of claim 10, wherein a linker is positioned between the binding domain and the Cas13 enzyme.

12. The engineered protein of claim 10, wherein the binding domain is fused to the N-terminus of the Cas13 enzyme, wherein the binding domain is an RNA binding domain selected from the group consisting of an hnRNP C RNA recognition motif (RRM) and an ADAR1 Z-DNA/RNA binding domain α (Zα).

13. The engineered protein of claim 12, wherein the engineered protein has a sequence at least 95% identical to SEQ ID NO: 22 or SEQ ID NO: 24.

14. The engineered protein of claim 1, wherein the binding domain is fused to the Cas13 enzyme within a structural loop of the Cas13 enzyme.

15-19. (canceled)

20. The engineered protein of claim 14, wherein the binding domain is inserted into the Cas13 enzyme (a) between amino acids corresponding to N415 and N416 of SEQ ID NO: 1; (b) between amino acids corresponding to N416 and K417 of SEQ ID NO: 1; or (c) between amino acids corresponding to K417 and G418 of SEQ ID NO: 1.

21-26. (canceled)

27. The engineered protein of claim 20, wherein the engineered protein has a sequence at least 95% identical to SEQ ID NO: 4 or SEQ ID NO: 5.

28. A method of enhancing the collateral activity of a Cas13 protein, the method comprising fusing the Cas13 protein to a binding domain, testing the collateral activity of the fusion protein, and identifying the fusion protein as having enhanced collateral activity if its collateral activity is increased as compared to a Cas13 enzyme.

29-51. (canceled)

52. A fusion protein made by the method of claim 28.

53. A composition comprising (i) the engineered Cas13 enzymes of claim 1 and (ii) a crRNA.

54-65. (canceled)

66. A method of detecting the presence of a target nucleic acid in a sample, the method comprising contacting the sample with the composition of claim 53 and detecting a signal from the detectable label.

67. The method of claim 66, wherein the sample is a biological sample or environmental sample.

68-90. (canceled)

91. The method of claim 66, wherein the method is performed in a buffer comprising 20-100 mM Tris pH 7.0-8.0 and Mg²⁺.

92. The method of claim 66, wherein the method is coupled with an electrochemical fluid chip.

93. The method of claim 66, wherein the method is coupled with a graphene field-effect transistor (gFET) biosensor.


SEQUENCE LISTING (145 sequences)

SEQ ID NO: 1 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 2 - protein, 1246 aa, synthetic construct
  REGION 1..1246: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNMMPNKVRKIGELVRYLNTNPVGGLLEYARSHGFAAEFKLVDQSGPPHEPKFVYQAKVGGRWFPAVCAHSKKQGKQEAADAALRVLIGENEKAERNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 3 - protein, 1256 aa, synthetic construct
  REGION 1..1256: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNMSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 4 - protein, 1251 aa, synthetic construct
  REGION 1..1251: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 5 - protein, 1257 aa, synthetic construct
  REGION 1..1257: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 6 - protein, 1251 aa, synthetic construct
  REGION 1..1251: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNSIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 7 - protein, 1229 aa, synthetic construct
  REGION 1..1229: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNLSSHFQELSIYQDQEQRILKFLEELGEGKATTAHDLSGKLGTPKKEINRVLYSLAKKGKLQKEAGTPPLWKIAVSTQNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 8 - protein, 1226 aa, synthetic construct
  REGION 1..1226: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNFLDMAEIKEKICDYLFNVSDSSALNLAKNIGLTKARDINAVLIDMERQGDVYRQGTTPPIWHLTDKKRERMQIKNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 9 - protein, 1251 aa, synthetic construct
  REGION 1..1251: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 10 - protein, 1257 aa, synthetic construct
  REGION 1..1257: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 11 - protein, 1251 aa, synthetic construct
  REGION 1..1251: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 12 - protein, 1257 aa, synthetic construct
  REGION 1..1257: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 13 - protein, 1251 aa, synthetic construct
  REGION 1..1251: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 14 - protein, 1257 aa, synthetic construct
  REGION 1..1257: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 15 - protein, 1259 aa, synthetic construct
  REGION 1..1259: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSGSSGPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRGSSGKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 16 - protein, 1265 aa, synthetic construct
  REGION 1..1265: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSGSSGASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGGSSGKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 17 - protein, 1281 aa, synthetic construct
  REGION 1..1281: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSGGGGSGGGGSGGGGSPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRGGGGSGGGGSGGGGSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 18 - protein, 1287 aa, synthetic construct
  REGION 1..1287: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSGGGGSGGGGSGGGGSASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGGGGGSGGGGSGGGGSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 19 - protein, 1278 aa, synthetic construct
  REGION 1..1278: note = Engineered sequence
MMPNKVRKIGELVRYLNTNPVGGLLEYARSHGFAAEFKLVDQSGPPHEPKFVYQAKVGGRWFPAVCAHSKKQGKQEAADAALRVLIGENEKAERSGGSSGGSSGSETPGTSESATPESSGGSSGGSMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 20 - protein, 1288 aa, synthetic construct
  REGION 1..1288: note = Engineered sequence
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVSGGSSGGSSGSETPGTSESATPESSGGSSGGSMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 21 - protein, 1283 aa, synthetic construct
  REGION 1..1283: note = Engineered sequence
PGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGGSSGGSSGSETPGTSESATPESSGGSSGGSMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 22 - protein, 1289 aa, synthetic construct
  REGION 1..1289: note = Engineered sequence
ASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGSGGSSGGSSGSETPGTSESATPESSGGSSGGSMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 23 - protein, 1283 aa, synthetic construct
  REGION 1..1283: note = Engineered sequence
SIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQSGGSSGGSSGSETPGTSESATPESSGGSSGGSMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 24 - protein, 1261 aa, synthetic construct
  REGION 1..1261: note = Engineered sequence
LSSHFQELSIYQDQEQRILKFLEELGEGKATTAHDLSGKLGTPKKEINRVLYSLAKKGKLQKEAGTPPLWKIAVSTQSGGSSGGSSGSETPGTSESATPESSGGSSGGSMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 25 - protein, 1258 aa, synthetic construct
  REGION 1..1258: note = Engineered sequence
FLDMAEIKEKICDYLFNVSDSSALNLAKNIGLTKARDINAVLIDMERQGDVYRQGTTPPIWHLTDKKRERMQIKSGGSSGGSSGSETPGTSESATPESSGGSSGGSMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 26 - protein, 1278 aa, synthetic construct
  REGION 1..1278: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALESGGSSGGSSGSETPGTSESATPESSGGSSGGSMMPNKVRKIGELVRYLNTNPVGGLLEYARSHGFAAEFKLVDQSGPPHEPKFVYQAKVGGRWFPAVCAHSKKQGKQEAADAALRVLIGENEKAER

SEQ ID NO: 27 - protein, 1288 aa, synthetic construct
  REGION 1..1288: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALESGGSSGGSSGSETPGTSESATPESSGGSSGGSMSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTV

SEQ ID NO: 28 - protein, 1283 aa, synthetic construct
  REGION 1..1283: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALESGGSSGGSSGSETPGTSESATPESSGGSSGGSPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGR

SEQ ID NO: 29 - protein, 1289 aa, synthetic construct
  REGION 1..1289: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALESGGSSGGSSGSETPGTSESATPESSGGSSGGSASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYG

SEQ ID NO: 30 - protein, 1283 aa, synthetic construct
  REGION 1..1283: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALESGGSSGGSSGSETPGTSESATPESSGGSSGGSSIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQ

SEQ ID NO: 31 - protein, 1261 aa, synthetic construct
  REGION 1..1261: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALESGGSSGGSSGSETPGTSESATPESSGGSSGGSLSSHFQELSIYQDQEQRILKFLEELGEGKATTAHDLSGKLGTPKKEINRVLYSLAKKGKLQKEAGTPPLWKIAVSTQ

SEQ ID NO: 32 - protein, 1258 aa, synthetic construct
  REGION 1..1258: note = Engineered sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALESGGSSGGSSGSETPGTSESATPESSGGSSGGSFLDMAEIKEKICDYLFNVSDSSALNLAKNIGLTKARDINAVLIDMERQGDVYRQGTTPPIWHLTDKKRERMQIK

SEQ ID NO: 33 - RNA, 105 nt, synthetic construct
  misc_feature 1..105: note = Engineered sequence
  source 1..105: mol_type = other RNA
gtctgataatggaccccaaaatcagcgaaatgcaccccgcattacgtttggtggaccctcagattcaactggcagtaaccagaatggagaacgcagtggggcgcg

SEQ ID NO: 34 - RNA, 227 nt, synthetic construct
  misc_feature 1..227: note = Engineered sequence
  source 1..227: mol_type = other RNA
gtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgt

SEQ ID NO: 35 - RNA, 173 nt, synthetic construct
  misc_feature 1..173: note = Engineered sequence
  source 1..173: mol_type = other RNA
ggccagtgaattcgagctcggtacccggggatcctctagaaatatggattacttggtagaacagcaatctactcgacctgcaggcatgcaagcttggcgtaatcatggtcatagctgtttcctgtgtttatccgctcacaattccacacaacatacgagccggaagcataaag

SEQ ID NO: 36 - RNA, 28 nt, synthetic construct
  misc_feature 1..28: note = Engineered sequence
  source 1..28: mol_type = other RNA
cgcattacgtttggtggaccctcagatt

SEQ ID NO: 37 - RNA, 28 nt, synthetic construct
  misc_feature 1..28: note = Engineered sequence
  source 1..28: mol_type = other RNA
cccattacgtttggtggaccctcagatt

SEQ ID NO: 38 - RNA, 28 nt, synthetic construct
  misc_feature 1..28: note = Engineered sequence
  source 1..28: mol_type = other RNA
cgcataacgtttggtggaccctcagatt

SEQ ID NO: 39 - RNA, 28 nt, synthetic construct
  misc_feature 1..28: note = Engineered sequence
  source 1..28: mol_type = other RNA
cgcattacgcttggtggaccctcagatt

SEQ ID NO: 40 - RNA, 28 nt, synthetic construct
  misc_feature 1..28: note = Engineered sequence
  source 1..28: mol_type = other RNA
cgcattacgtttgctggaccctcagatt

SEQ ID NO: 41 - RNA, 28 nt, synthetic construct
  misc_feature 1..28: note = Engineered sequence
  source 1..28: mol_type = other RNA
cgcattacgtttggtggtccctcagatt

SEQ ID NO: 42 - RNA, 28 nt, synthetic construct
  misc_feature 1..28: note = Engineered sequence
  source 1..28: mol_type = other RNA
cgcattacgtttggtggacccacagatt

SEQ ID NO: 43 - RNA, 28 nt, synthetic construct
  misc_feature 1..28: note = Engineered sequence
  source 1..28: mol_type = other RNA
cgcattacgtttggtggaccctcagttt

SEQ ID NO: 44 - RNA, 28 nt, synthetic construct
  misc_feature 1..28: note = Engineered sequence
  source 1..28: mol_type = other RNA
gccattacgtttggtggaccctcagatt

SEQ ID NO: 45 - RNA, 28 nt, synthetic construct
  misc_feature 1..28: note = Engineered sequence
  source 1..28: mol_type = other RNA
cgcaaaacgtttggtggaccctcagatt

SEQ ID NO: 46 - RNA, 28 nt, synthetic construct
  misc_feature 1..28: note = Engineered sequence
  source 1..28: mol_type = other RNA
cgcattaccattggtggaccctcagatt

SEQ ID NO: 47 - RNA, 28 nt, synthetic construct
  misc_feature 1..28: note = Engineered sequence
  source 1..28: mol_type = other RNA
cgcattacgtttcctggaccctcagatt

SEQ ID NO: 48 - RNA, 28 nt, synthetic construct
  misc_feature 1..28: note = Engineered sequence
  source 1..28: mol_type = other RNA
cgcattacgtttggtgctccctcagatt

SEQ ID NO: 49 - RNA, 28 nt, synthetic construct
  misc_feature 1..28: note = Engineered sequence
  source 1..28: mol_type = other RNA
cgcattacgtttggtggaccgacagatt

SEQ ID NO: 50 - RNA, 28 nt, synthetic construct
  misc_feature 1..28: note = Engineered sequence
  source 1..28: mol_type = other RNA
cgcattacgtttggtggaccctcacttt

SEQ ID NO: 51 - RNA, 334 nt, synthetic construct
  misc_feature 1..334: note = Engineered sequence
  source 1..334: mol_type = other RNA
agctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagcagtctccagcgccata

SEQ ID NO: 52 - RNA, 170 nt, synthetic construct
  misc_feature 1..170: note = Engineered sequence
  source 1..170: mol_type = other RNA
agtacatattcaggggccaacctctcaacaatgacgaagaccatgctcactggacagaagcaaaaatgctgctggacaacatcaacacaccagaagggattataccagctctctttgaaccagaaagggagaagtcagccgccatagacggtgaataccgcctgaagggt

SEQ ID NO: 53 - RNA, 173 nt, synthetic construct
  misc_feature 1..173: note = Engineered sequence
  source 1..173: mol_type = other RNA
gaggaaaataacattgcaaaggtggagcctatggtttagttatcttgatcattgtgataatatcctggcggaggctttaacccaaataacttgcacagttgatttagcacagattctgagggaatattcatgggctcatattttagagggaagacctcttattggagccacac

SEQ ID NO: 54 - RNA, 23 nt, synthetic construct
  misc_feature 1..23: note = Engineered sequence
  source 1..23: mol_type = other RNA
tgtgcaaatccatgcaaaactga

SEQ ID NO: 55 - RNA, 20 nt, synthetic construct
  misc_feature 1..20: note = Engineered sequence
  source 1..20: mol_type = other RNA
taggatgggggtgagaggtg

SEQ ID NO: 56 - RNA, 58 nt, synthetic construct
  misc_feature 1..58: note = Engineered sequence
  source 1..58: mol_type = other RNA
gactaccccaaaaacgaaggggactaaaacaatctgagggtccaccaaacgtaatgcg

SEQ ID NO: 57 - DNA, 48 bp, synthetic construct
  misc_feature 1..48: note = Engineered sequence
  source 1..48: mol_type = other DNA
gacaccccaaaaacgaaggggacaaaaccaccaaacgagcacaccaag

SEQ ID NO: 58 - RNA, 58 nt, synthetic construct
  misc_feature 1..58: note = Engineered sequence
  source 1..58: mol_type = other RNA
gactaccccaaaaacgaaggggactaaaactagattgctgttctaccaagtaatccat

SEQ ID NO: 59 - RNA, 58 nt, synthetic construct
  misc_feature 1..58: note = Engineered sequence
  source 1..58: mol_type = other RNA
gactaccccaaaaacgaaggggactaaaacccgggtaccgagctcgaattcactggcc

SEQ ID NO: 60 - RNA, 58 nt, synthetic construct
  misc_feature 1..58: note = Engineered sequence
  source 1..58: mol_type = other RNA
gactaccccaaaaacgaaggggactaaaactttctagaggatccccgggtaccgagct

SEQ ID NO: 61 - RNA, 58 nt, synthetic construct
  misc_feature 1..58: note = Engineered sequence
  source 1..58: mol_type = other RNA
gactaccccaaaaacgaaggggactaaaacccaagtaatccatatttctagaggatcc

SEQ ID NO: 62 - RNA, 58 nt, synthetic construct
  misc_feature 1..58: note = Engineered sequence
  source 1..58: mol_type = other RNA
gactaccccaaaaacgaaggggactaaaacagattgctgttctaccaagtaatccata

SEQ ID NO: 63 - RNA, 58 nt, synthetic construct
  misc_feature 1..58: note = Engineered sequence
  source 1..58: mol_type = other RNA
gactaccccaaaaacgaaggggactaaaaccctgcaggtcgagtagattgctgttcta

SEQ ID NO: 64 - RNA, 58 nt, synthetic construct
  misc_feature 1..58: note = Engineered sequence
  source 1..58: mol_type = other RNA
gactaccccaaaaacgaaggggactaaaacgccaagcttgcatgcctgcaggtcgagt

SEQ ID NO: 65 - RNA, 58 nt, synthetic construct
  misc_feature 1..58: note = Engineered sequence
  source 1..58: mol_type = other RNA
gactaccccaaaaacgaaggggactaaaacatgaccatgattacgccaagcttgcatg

SEQ ID NO: 66 - RNA, 58 nt, synthetic construct
  misc_feature 1..58: note = Engineered sequence
  source 1..58: mol_type = other RNA
gactaccccaaaaacgaaggggactaaaaccacaggaaacagctatgaccatgattac

SEQ ID NO: 67 - RNA, 58 nt, synthetic construct
  misc_feature 1..58: note = Engineered sequence
  source 1..58: mol_type = other RNA
gactaccccaaaaacgaaggggactaaaactgtgagcggataaacacaggaaacagct

SEQ ID NO: 68 - RNA, 58 nt, synthetic construct
  misc_feature 1..58: note = Engineered sequence
  source 1..58: mol_type = other RNA
gactaccccaaaaacgaaggggactaaaacatgttgtgtggaattgtgagcggataaa

SEQ ID NO: 69 - RNA, 58 nt, synthetic construct
  misc_feature 1..58: note = Engineered sequence
  source 1..58: mol_type = other RNA
gactaccccaaaaacgaaggggactaaaactgcttccggctcgtatgttgtgtggaat

SEQ ID NO: 70 - RNA, 58 nt, synthetic construct
  misc_feature 1..58: note = Engineered sequence
  source 1..58: mol_type = other RNA
gactaccccaaaaacgaaggggactaaaaccatgtagtgcgccacgagcaaaatgatg

SEQ ID NO: 71 - RNA, 58 nt, synthetic construct
  misc_feature 1..58: note = Engineered sequence
  source 1..58: mol_type = other RNA
gactaccccaaaaacgaaggggactaaaacggtataatcccttctggtgtgttgatgt

SEQ ID NO: 72 - RNA, 58 nt, synthetic construct
  misc_feature 1..58: note = Engineered sequence
  source 1..58: mol_type = other RNA
gactaccccaaaaacgaaggggactaaaacatcaactgtgcaagttatttgggttaaa

SEQ ID NO: 73 - RNA, 53 nt, synthetic construct
  misc_feature 1..53: note = Engineered sequence
  source 1..53: mol_type = other RNA
gactaccccaaaaacgaaggggactaaaactcagttttgcatggatttgcaca

SEQ ID NO: 74 - RNA, 50 nt, synthetic construct
  misc_feature 1..50: note = Engineered sequence
  source 1..50: mol_type = other RNA
gactaccccaaaaacgaaggggactaaaaccacctctcacccccatccta

SEQ ID NO: 75 - skipped

SEQ ID NO: 76 - RNA, 11 nt, synthetic construct
  misc_feature 1..11: note = Engineered sequence
  source 1..11: mol_type = other RNA
ttttttttttt

SEQ ID NO: 77 - RNA, 15 nt, synthetic construct
  misc_feature 1..15: note = Engineered sequence
  source 1..15: mol_type = other RNA
ttttttttttttttt

SEQ ID NO: 78 - RNA, 20 nt, synthetic construct
  misc_feature 1..20: note = Engineered sequence
  source 1..20: mol_type = other RNA
tttttttttttttttttttt

SEQ ID NO: 79 - DNA, 54 bp, synthetic construct
  misc_feature 1..54: note = Engineered sequence
  source 1..54: mol_type = other DNA
gaaattaatacgactcactatagggactaccccaaaaacgaaggggactaaaac

SEQ ID NO: 80 - DNA, 54 bp, synthetic construct
  misc_feature 1..54: note = Engineered sequence
  source 1..54: mol_type = other DNA
ggccagtgaattcgagctcggtacccgggttttagtccccttcgtttttggggt

SEQ ID NO: 81 - DNA, 54 bp, synthetic construct
  misc_feature 1..54: note = Engineered sequence
  source 1..54: mol_type = other DNA
agctcggtacccggggatcctctagaaagttttagtccccttcgtttttggggt

SEQ ID NO: 82 - DNA, 54 bp, synthetic construct
  misc_feature 1..54: note = Engineered sequence
  source 1..54: mol_type = other DNA
ggatcctctagaaatatggattacttgggttttagtccccttcgtttttggggt

SEQ ID NO: 83 - DNA, 54 bp, synthetic construct
  misc_feature 1..54: note = Engineered sequence
  source 1..54: mol_type = other DNA
tatggattacttggtagaacagcaatctgttttagtccccttcgtttttggggt

SEQ ID NO: 84 - DNA, 54 bp, synthetic construct
  misc_feature 1..54: note = Engineered sequence
  source 1..54: mol_type = other DNA
tagaacagcaatctactcgacctgcagggttttagtccccttcgtttttggggt

SEQ ID NO: 85 - DNA, 54 bp, synthetic construct
  misc_feature 1..54: note = Engineered sequence
  source 1..54: mol_type = other DNA
actcgacctgcaggcatgcaagcttggcgttttagtccccttcgtttttggggt

SEQ ID NO: 86 - DNA, 54 bp, synthetic construct
  misc_feature 1..54: note = Engineered sequence
  source 1..54: mol_type = other DNA
catgcaagcttggcgtaatcatggtcatgttttagtccccttcgtttttggggt

SEQ ID NO: 87 - DNA, 54 bp, synthetic construct
  misc_feature 1..54: note = Engineered sequence
  source 1..54: mol_type = other DNA
gtaatcatggtcatagctgtttcctgtggttttagtccccttcgtttttggggt

SEQ ID NO: 88 - DNA, 54 bp, synthetic construct
  misc_feature 1..54: note = Engineered sequence
  source 1..54: mol_type = other DNA
agctgtttcctgtgtttatccgctcacagttttagtccccttcgtttttggggt

SEQ ID NO: 89 - DNA, 54 bp, synthetic construct
  misc_feature 1..54: note = Engineered sequence
  source 1..54: mol_type = other DNA
tttatccgctcacaattccacacaacatgttttagtccccttcgtttttggggt

SEQ ID NO: 90 - DNA, 54 bp, synthetic construct
  misc_feature 1..54: note = Engineered sequence
  source 1..54: mol_type = other DNA
attccacacaacatacgagccggaagcagttttagtccccttcgtttttggggt

SEQ ID NO: 91 - DNA, 54 bp, synthetic construct
  misc_feature 1..54: note = Engineered sequence
  source 1..54: mol_type = other DNA
catcattttgctcgtggcgcactacatggttttagtccccttcgtttttggggt

SEQ ID NO: 92 - DNA, 54 bp, synthetic construct
  misc_feature 1..54: note = Engineered sequence
  source 1..54: mol_type = other DNA
acatcaacacaccagaagggattataccgttttagtccccttcgtttttggggt

SEQ ID NO: 93 - DNA, 54 bp, synthetic construct
  misc_feature 1..54: note = Engineered sequence
  source 1..54: mol_type = other DNA
tttaacccaaataacttgcacagttgatgttttagtccccttcgtttttggggt

SEQ ID NO: 94 - DNA, 49 bp, synthetic construct
  misc_feature 1..49: note = Engineered sequence
  source 1..49: mol_type = other DNA
tgtgcaaatccatgcaaaactgagttttagtccccttcgtttttggggt

SEQ ID NO: 95 - DNA, 46 bp, synthetic construct
  misc_feature 1..46: note = Engineered sequence
  source 1..46: mol_type = other DNA
taggatgggggtgagaggtggttttagtccccttcgtttttggggt

SEQ ID NO: 96 - DNA, 25 bp, synthetic construct
  misc_feature 1..25: note = Engineered sequence
  source 1..25: mol_type = other DNA
gaaattaatacgactcactataggg

SEQ ID NO: 97 - DNA, 20 bp, synthetic construct
  misc_feature 1..20: note = Engineered sequence
  source 1..20: mol_type = other DNA
cgcgccccactgcgttctcc

SEQ ID NO: 98 - DNA, 20 bp, synthetic construct
  misc_feature 1..20: note = Engineered sequence
  source 1..20: mol_type = other DNA
ctttatgcttccggctcgta

SEQ ID NO: 99 - DNA, 28 bp, synthetic construct
  misc_feature 1..28: note = Engineered sequence
  source 1..28: mol_type = other DNA
acaaatgttaaaaacactattagcataa

SEQ ID NO: 100 - DNA, 25 bp, synthetic construct
  misc_feature 1..25: note = Engineered sequence
  source 1..25: mol_type = other DNA
gaaattaatacgactcactataggc

SEQ ID NO: 101 - DNA, 52 bp, synthetic construct
  misc_feature 1..52: note = Engineered sequence
  source 1..52: mol_type = other DNA
aatctgagggtccaccaaacgtaatgcgcctatagtgagtcgtattaatttc

SEQ ID NO: 102 - DNA, 52 bp, synthetic construct
  misc_feature 1..52: note = Engineered sequence
  source 1..52: mol_type = other DNA
aatctgagggtccaccaaacgtaatgggcctatagtgagtcgtattaatttc

SEQ ID NO: 103 - DNA, 52 bp, synthetic construct
  misc_feature 1..52: note = Engineered sequence
  source 1..52: mol_type = other DNA
aatctgagggtccaccaaacgttatgcgcctatagtgagtcgtattaatttc

SEQ ID NO: 104 - DNA, 52 bp, synthetic construct
  misc_feature 1..52: note = Engineered sequence
  source 1..52: mol_type = other DNA
aatctgagggtccaccaagcgtaatgcgcctatagtgagtcgtattaatttc

SEQ ID NO: 105 - DNA, 52 bp, synthetic construct
  misc_feature 1..52: note = Engineered sequence
  source 1..52: mol_type = other DNA
aatctgagggtccagcaaacgtaatgcgcctatagtgagtcgtattaatttc

SEQ ID NO: 106 - DNA, 52 bp, synthetic construct
  misc_feature 1..52: note = Engineered sequence
  source 1..52: mol_type = other DNA
aatctgagggaccaccaaacgtaatgcgcctatagtgagtcgtattaatttc

SEQ ID NO: 107 - DNA, 52 bp, synthetic construct
  misc_feature 1..52: note = Engineered sequence
  source 1..52: mol_type = other DNA
aatctgtgggtccaccaaacgtaatgcgcctatagtgagtcgtattaatttc

SEQ ID NO: 108 - DNA, 52 bp, synthetic construct
  misc_feature 1..52: note = Engineered sequence
  source 1..52: mol_type = other DNA
aaactgagggtccaccaaacgtaatgcgcctatagtgagtcgtattaatttc

SEQ ID NO: 109 - DNA, 52 bp, synthetic construct
  misc_feature 1..52: note = Engineered sequence
  source 1..52: mol_type = other DNA
aatctgagggtccaccaaacgtaatggccctatagtgagtcgtattaatttc

SEQ ID NO: 110 - DNA, 52 bp, synthetic construct
  misc_feature 1..52: note = Engineered sequence
  source 1..52: mol_type = other DNA
aatctgagggtccaccaaacgttttgcgcctatagtgagtcgtattaatttc

SEQ ID NO: 111 - DNA, 52 bp, synthetic construct
  misc_feature 1..52: note = Engineered sequence
  source 1..52: mol_type = other DNA
aatctgagggtccaccaatggtaatgcgcctatagtgagtcgtattaatttc

SEQ ID NO: 112 - DNA, 52 bp, synthetic construct
  misc_feature 1..52: note = Engineered sequence
  source 1..52: mol_type = other DNA
aatctgagggtccaggaaacgtaatgcgcctatagtgagtcgtattaatttc

SEQ ID NO: 113 - DNA, 52 bp, synthetic construct
  misc_feature 1..52: note = Engineered sequence
  source 1..52: mol_type = other DNA
aatctgagggagcaccaaacgtaatgcgcctatagtgagtcgtattaatttc

SEQ ID NO: 114 - DNA, 52 bp, synthetic construct
  misc_feature 1..52: note = Engineered sequence
  source 1..52: mol_type = other DNA
aatctgtcggtccaccaaacgtaatgcgcctatagtgagtcgtattaatttc

SEQ ID NO: 115 - DNA, 52 bp, synthetic construct
  misc_feature 1..52: note = Engineered sequence
  source 1..52: mol_type = other DNA
aaagtgagggtccaccaaacgtaatgcgcctatagtgagtcgtattaatttc

SEQ ID NO: 116 - DNA, 22 bp, synthetic construct
  misc_feature 1..22: note = Engineered sequence
  source 1..22: mol_type = other DNA
tatggcgctggagactgctact

SEQ ID NO: 117 - DNA, 48 bp, synthetic construct
  misc_feature 1..48: note = Engineered sequence
  source 1..48: mol_type = other DNA
gaaattaatacgactcactatagggagtacatattcaggggccaacct

SEQ ID NO: 118 - DNA, 21 bp, synthetic construct
  misc_feature 1..21: note = Engineered sequence
  source 1..21: mol_type = other DNA
acccttcaggcggtattcacc

SEQ ID NO: 119 - DNA, 52 bp, synthetic construct
  misc_feature 1..52: note = Engineered sequence
  source 1..52: mol_type = other DNA
gaaattaatacgactcactataggggaggaaaataacattgcaaaggtggag

SEQ ID NO: 120 - DNA, 24 bp, synthetic construct
  misc_feature 1..24: note = Engineered sequence
  source 1..24: mol_type = other DNA
gtgtggctccaataagaggtcttc

SEQ ID NO: 121 - protein, 1366 aa, synthetic construct
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGGSGGSGGSASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 122 - protein, 1366 aa, synthetic construct
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGSGGSGGSGGSPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 123 - protein, 4 aa, synthetic construct
GSSG

SEQ ID NO: 124 - protein, 15 aa, synthetic construct
GGGGSGGGGSGGGGS

SEQ ID NO: 125 - protein, 10 aa, synthetic construct
SGGSGGSGGS

SEQ ID NO: 126 - RNA, 28 nt, synthetic construct
  source 1..28: mol_type = other RNA
cgctttacgtttggtggaccctcagatt

SEQ ID NO: 127 - RNA, 28 nt, synthetic construct
  source 1..28: mol_type = other RNA
cgcattaggtttggtggaccctcagatt

SEQ ID NO: 128 - RNA, 28 nt, synthetic construct
  source 1..28: mol_type = other RNA
cgcattacgttaggtggaccctcagatt

SEQ ID NO: 129 - RNA, 28 nt, synthetic construct
  source 1..28: mol_type = other RNA
cgcattacgtttggtcgaccctcagatt

SEQ ID NO: 130 - RNA, 28 nt, synthetic construct
  source 1..28: mol_type = other RNA
cgcattacgtttggtggacgctcagatt

SEQ ID NO: 131 - RNA, 28 nt, synthetic construct
  source 1..28: mol_type = other RNA
cgcattacgtttggtggaccctctgatt

SEQ ID NO: 132 - RNA, 28 nt, synthetic construct
  source 1..28: mol_type = other RNA
cgcattacgtttggtggaccctcagata

SEQ ID NO: 133 - RNA, 36 nt, synthetic construct
  source 1..36: mol_type = other RNA
accccgcattacgtttggtggaccctcagattcaac

SEQ ID NO: 134 - DNA, 52 bp, synthetic construct
  source 1..52: mol_type = other DNA
aatcagagggtccaccaaacgtaatgcgcctatagtgagtcgtattaatttc

SEQ ID NO: 135 - DNA, 52 bp, synthetic construct
  source 1..52: mol_type = other DNA
tatctgagggtccaccaaacgtaatgcgcctatagtgagtcgtattaatttc

SEQ ID NO: 136 - DNA, 52 bp, synthetic construct; identical to SEQ ID NO: 109
  source 1..52: mol_type = other DNA

SEQ ID NO: 137 - DNA, 52 bp, synthetic construct; identical to SEQ ID NO: 110
  source 1..52: mol_type = other DNA

SEQ ID NO: 138 - DNA, 52 bp, synthetic construct; identical to SEQ ID NO: 111
  source 1..52: mol_type = other DNA

SEQ ID NO: 139 - DNA, 52 bp, synthetic construct; identical to SEQ ID NO: 112
  source 1..52: mol_type = other DNA

SEQ ID NO: 140 - DNA, 52 bp, synthetic construct; identical to SEQ ID NO: 113
  source 1..52: mol_type = other DNA

SEQ ID NO: 141 - DNA, 60 bp, synthetic construct
  source 1..60: mol_type = other DNA
gaaattaatacgactcactataggaccccgcattacgtttggtggaccctcagattcaac

SEQ ID NO: 142 - DNA, 60 bp, synthetic construct
  source 1..60: mol_type = other DNA
gttgaatctgagggtccaccaaacgtaatgcggggtcctatagtgagtcgtattaatttc

SEQ ID NO: 143 - DNA, 20 bp, synthetic construct
  source 1..20: mol_type = other DNA
gaccccaaaatcagcgaaat

SEQ ID NO: 144 - DNA, 24 bp, synthetic construct
  source 1..24: mol_type = other DNA
tctggttactgccagttgaatctg

SEQ ID NO: 145 - DNA, 24 bp, synthetic construct
  source 1..24: mol_type = other DNA
accccgcattacgtttggtggacc